(19) United States
(12) Patent Application Publication
(10) Pub. No.: US 2009/0317335 A1
(43) Pub. Date: Dec. 24, 2009

(54) Hybrid Nanomaterials as Multimodal Imaging Contrast Agents

(76) Inventors: Wenbin Lin, Chapel Hill, NC (US); William Rieter, Charleston, SC (US); Kathryn Taylor, Chapel Hill, NC (US); Jason Kim, Chapel Hill, NC (US)

Correspondence Address: JENKINS, WILSON, TAYLOR & HUNT, P. A., Suite 1200 UNIVERSITY TOWER, 3100 TOWER BLVD.,, DURHAM, NC, 27707, US

(21) Appl. No.: 12/226,499

(22) PCT Filed: Apr. 20, 2007
(86) PCT No.: PCT/US07/09796

§ 371 Date: Jul. 7, 2009

Related U.S. Application Data
(60) Provisional application No. 60/793,454, filed on Apr. 20, 2006.
(60) Provisional application No. 60/906,793, filed on Mar. 13, 2007.

(51) Int. Cl. A61B 5/055; A61K 49/18; G01R 33/44

(52) U.S. Cl. 424/9.323; 324/307; 977/810

(57) ABSTRACT

The presently disclosed subject matter provides hybrid nanomaterials for use as magnetic resonance imaging (MRI), optical and/or multimodal contrast imaging agents. The hybrid nanomaterials comprise a polymeric matrix material and a plurality of coordination complexes, each coordination complex comprising a functionalized chelating group and a paramagnetic metal ion. The nanoparticle can further comprise a luminophore. Methods of synthesizing and using the nanoparticles are provided. The nanoparticles can be used to diagnose diseases, including cancer, cardiovascular disease, and diseases related to inflammation.

### RELATED APPLICATIONS

The presently disclosed subject matter claims the benefit of U.S. Provisional Patent Application Ser. No. 60/793,454, filed Apr. 20, 2006; and U.S. Provisional Patent Application Ser. No. 60/906,793, filed Mar. 13, 2007; the disclosure of each of which is incorporated herein by reference in its entirety.

### GOVERNMENT INTEREST

The presently disclosed subject matter was made with U.S. Government support from the National Science Foundation (Grant No. CHE-0512495) and the U.S. National Institutes of Health (Grant Nos U54-CA119343 and P20 RR020764). The U.S. Government has certain rights in the invention.

### TECHNICAL FIELD

The presently disclosed subject matter relates to hybrid nanomaterials, the synthesis of hybrid nanomaterials, and their use as magnetic resonance imaging (MRI), optical and/or multimodal imaging contrast agents. The hybrid nanomaterials can comprise inorganic and/or organic polymeric matrix materials along with paramagnetic and/or luminescent groups. The nanomaterials can further include targeting agents to direct the nanomaterials to specific sites for use in disease diagnosis and imaging.

### Abbreviations

δ=chemical shift
° C.=degrees Celsius
APS=3-aminopropyl triethoxysilane or trimethoxysilane
bpy=2,2′-bipyridine
calcd=calculated
cm=centimeters
CTAB=cetyltrimethyl ammonium bromide
DCP=direct current plasma
DMSO=dimethyl sulfoxide
DOTA=1,4,7,10-tetraazacyclododecane-1,4,7,10-tetraacetic acid
DTPA=diethylenetriamine pentaacetate
DTTA=diethylenetriamine tetraacetate
ESI=electrospray ionization
FITC=fluorescein isothiocyanate
g=grams
Gd=gadolinium
hr=hours
Hz=hertz
kg=kilograms
LbL=layer-by-layer
MeOH=methanol
MHz=megahertz
min.=minutes
mL=milliliters
mm=millimeters
mM=millimolar
mmol=millimole
m.p.=melting point
MRI=magnetic resonance imaging
ms=millisecond
MS=mass spectroscopy
MTS=3-(4,5-dimethylthiazol-2-yl)-5-(3-carboxy-methoxyphenyl)-2-(4-sulfophenyl)-2H-tetrazolium
M_{w}=molecular weight
MWCO=molecular weight cut off
NMR=nuclear magnetic resonance
PEG=polyethylene glycol
PEO=polyethylene oxide
PLA=poly(lactic acid)
PSS=poly(styrene sulfonate)
rpm=revolutions per minute
Ru(bpy)₃²⁺=ruthenium(II) tris(2,2′-bipyridine)
s=seconds
SEM=scanning electron microscope
Si=silicon
SNP=silica nanoparticles
TEM=transmission electron microscope
TEOS=tetraethyl orthosilicate
TGA=thermogravimetric analysis
TMEDA=tetramethylethane diamine
TMPTA=trimethylolpropane triacrylate
TMS=tetramethylsilane
w-=[water]/[surfactant]

### BACKGROUND

Magnetic resonance imaging (MRI) has become a useful tool for diagnosis and research. MRI has proven particularly useful in the field of medicine to detect and diagnose disease states and tissue abnormalities. The current technology relies on detecting the energy emitted when the hydrogen nuclei in the water contained in tissues and body fluids returns to a ground state subsequent to excitation with a radio frequency. Observation of this phenomenon depends on imposing a magnetic field across the area to be observed, so that the distribution of hydrogen nuclear spins is statistically oriented in alignment with the magnetic field, and then imposing an appropriate radio frequency. This results in an excited state in which this statistical alignment is disrupted. The decay of the distribution to the ground state can then be measured as an emission of energy, the pattern of which can be detected as an image.

While the above described process is theoretically possible, it turns out that the relaxation rate of the relevant hydrogen nuclei, left to their own devices, is too slow to generate detectable amounts of energy, as a practical matter. In order to remedy this, the area to be imaged is supplied with a contrast agent, generally a strongly paramagnetic metal, which effectively acts as a catalyst to accelerate the decay, thus permitting sufficient energy to be emitted to create a detectable bright signal. To put it succinctly, MRI contrast agents decrease the relaxation time and increase the reciprocal of the relaxation time—i.e., the “relaxivity” of the surrounding hydrogen nuclei.

Two types of relaxation times can be measured. T₁ is the time for the magnetic distribution to return to 63% of its original distribution longitudinally with respect to the magnetic field. T₂ measures the time wherein 63% of the distribution returns to the ground state transverse to the magnetic field. Paramagnetic metal ions, as a result of their unpaired electrons, act as potent relaxation enhancement agents, increasing tissue intensity on T₁-weighted images. The mechanism of T₁ relaxation is generally a through space dipole-dipole interaction between the unpaired electrons of the paramagnet (i.e., the metal atom with an unpaired electron) and bulk water molecules (i.e., water molecules that are not “bound” to the metal atom) that are in fast exchange with water molecules in the metal's inner coordination sphere (i.e., water molecules that are bound to the metal atom). The efficiency of a paramagnetic metal complex contrast agent can be expressed by its relaxivity (r₁ and/or r₂).

The lanthanide atom Gd³⁺ is the most frequently chosen metal atom for MRI contrast agents because it has a very high magnetic moment and a symmetric electronic ground state. Transition metals, including but not limited to high spin Mn(II) and Fe(III), also are candidates for use in MRI agents, due to their high magnetic moments.

Gd³⁺ has seven unpaired electrons, which gives it the greatest power of any metal ion to shift the MRI signal of the proton in H₂O. Gd³⁺ itself is toxic, however. A suitable ligand or chelator must therefore be used to complex the Gd³⁺, thereby preventing it from exerting its toxic effect. Common ligands used for gadolinium-based MRI contrast agents include diethylenetriaminepenta-acetate (DTPA) and 1,4,7,10-tetraazacyclododecane-1,4,7,10-tetraacetic acid (DOTA). Unfortunately, a drawback with commonly used contrast agents, such as the DTPA complex of Gd³⁺, is that a relatively large amount of the complex (e.g., about 7 g) is typically injected per patient to produce a good contrast.

Thus, there exists a need in the art for new MRI contrast agents with enhanced efficiency that could be used in smaller doses. Such higher efficiency MRI agents could also be readily functionizable so that they could include optical imaging agents and/or could be conjugated to antibodies or other targeting agents to provide improved MRI agents for specific purposes.

### SUMMARY

The presently disclosed subject matter provides a contrast agent for magnetic resonance imaging (MRI) comprising a hybrid nanoparticle, said hybrid nanoparticle comprising a polymeric matrix material and a plurality of coordination complexes, each coordination complex comprising a functionalized chelating group and a paramagnetic metal ion.

In some embodiments, the contrast agent comprises at least one luminophore for optical imaging. In some embodiments, the luminophore is a fluorophore. In some embodiments, the fluorophore is selected from the group consisting of ruthenium(II) tris(2,2′-bipyridine) (Ru(bpy)₃²⁺) and fluoroscein isothiocyanate (FITC).

In some embodiments, the luminophore is embedded in the hybrid nanoparticle. In some embodiments, the luminophore is bound to a surface of the hybrid nanoparticle.

In some embodiments, the polymeric matrix material is an inorganic polymer. In some embodiments, the inorganic polymer comprises silicon. In some embodiments, the inorganic polymer comprises SiO₂.

In some embodiments, the polymeric matrix material comprises an organic polymer. In some embodiments, the organic polymer is selected from the group consisting of polyacrylic acid and polylactide.

In some embodiments, the polymeric matrix material is biodegradable. In some embodiments, the polymeric matrix material is non-biodegradable.

In some embodiments, the paramagnetic metal ion comprises an element selected from the group consisting of a transition element, a lanthanide and an actinide. In some embodiments, the paramagnetic metal ion comprises an element selected from the group consisting of scandium, titanium, vanadium, chromium, manganese, iron, cobalt, nickel, copper, molybdenum, ruthenium, cerium, praseodymium, neodymium, promethium, samarium, europium, gadolinium, terbium, dysprosium, holmium, erbium, thulium, and ytterbium. In some embodiments, the paramagnetic metal ion is selected from the group consisting of gadolinium(III) and manganese(II).

In some embodiments, the functionalized chelating group comprises a polyaminocarboxylate metal chelating ligand or a polyaminophosphonate metal chelating ligand. In some embodiments, the metal chelating ligand comprises a ligand selected from the group consisting of diethylenetriamine tetraacetate (DTTA), diethylenetriamine pentaacetate (DTPA), and 1,4,7,10-tetraazacyclododecane-1,4,7,10-tetraacetic acid (DOTA).

In some embodiments, the functionalized chelating group is functionalized by at least one reactive moiety that can covalently bond to the polymeric matrix material or to another functionalized chelating group. In some embodiments, the reactive moiety is selected from the group consisting of vinyl, siloxy, and combinations thereof. In some embodiments, the functionalized chelating group is functionalized by more than one reactive moiety. In some embodiments, the functionalized chelating group is selected from aminopropyl(trimethoxysilyl)diethylenetriamine tetraacetate, bis(aminopropyl-triethoxysilyl)diethylenetriamine pentaacetate, bis(2-aminoethylmethacrylate)-diethylenetriamine pentaacetic acid, bis(aminopropyltrimethoxysilyl)-1,4,7,10-tetraazacyclododecane-1,4,7,10-tetraacetic acid, and aminopropyl(trimethoxysilyl)-1,4,7,10-tetraazacyclododecane-1,4,7,10-tetraacetic acid.

In some embodiments, the functionalized chelating group further comprises at least one biodegradable linkage. In some embodiments, the biodegradable linkage is disulfide.

In some embodiments, the polymeric matrix material and the plurality of coordination complexes form a copolymer. The plurality of functionalized coordination complexes can be dispersed throughout the copolymer and/or can form a polymeric layer disposed over a core polymeric layer comprising the polymeric matrix material. In some embodiments, one or more of the plurality of coordination complexes is bound to a surface of the nanoparticle.

In some embodiments, the nanoparticle further comprises one or more anionic groups. In some embodiments, the anionic groups are sulfonate groups. In some embodiments, the nanoparticle comprises a layer comprising anionic groups. In some embodiments, the layer comprises poly(styrene sulfonate) (PSS).

In some embodiments, the contrast agent comprises a plurality of layers, the layers comprising a first layer comprising the polymeric matrix material and at least some of the plurality of coordination complexes; and a second layer disposed over the first layer, said second layer comprising at least some of the plurality of coordination complexes.

In some embodiments, the layered contrast agent further comprises a third layer disposed over the second layer, said third layer comprising anionic groups. In some embodiments, the third layer comprises poly(styrene sulfonate) (PSS). In some embodiments, the layered contrast agent can comprise a fourth layer disposed over the third layer, said fourth layer comprising at least some of the plurality of coordination complexes.

In some embodiments, the layered contrast agent comprising four layers can comprise one or more additional layers comprising some of the plurality of coordination complexes and one or more additional layers comprising anionic groups, said additional layers being disposed such that each layer comprising some of the plurality of coordination complexes is the outermost layer of the nanoparticle and is disposed over a layer of anionic groups or is an inner layer of the nanoparticle and is disposed between two layers of anionic groups; and wherein each layer comprising anionic groups is either the outermost layer of the nanoparticle and is disposed over a layer comprising some of the plurality of coordination complexes or is an inner layer of the nanoparticle and is disposed between two layers, each comprising some of the plurality of coordination complexes.

In some embodiments, the nanoparticle is spherical.

In some embodiments, the nanoparticle has a diameter of about 100 nm or less. In some embodiments, the diameter is about 50 nm or less.

In some embodiments, the contrast agent comprises an additional moiety bound to a surface of the nanoparticle, said additional moiety selected from the group consisting of a targeting agent, a solubility-enhancing agent, a circulation half-life enhancing agent, and a combination thereof. In some embodiments, the additional moiety is a targeting agent selected from the group consisting of an antibody, an antibody fragment, or a peptide. In some embodiments, the targeting agent is an anti-major histocompatibility complex (MHC)-II antibody. In some embodiments, the targeting agent targets a tumor.

In some embodiments, the additional moiety comprises a polyethylene glycol (PEG)-based polymer. In some embodiments, the PEG-based polymer is polyethylene oxide (PEO)-500.

In some embodiments, the nanoparticle comprises at least one thousand paramagnetic metal ions. In some embodiments, the nanoparticle comprises at least 25,000 paramagnetic metal ions. In some embodiments, the nanoparticle comprises at least 60,000 paramagnetic metal ions.

In some embodiments, the contrast agent has a longitudinal relaxivity (r1) of about 7.0 mmol⁻¹ s⁻¹ or greater, calculated based on metal ion concentration. In some embodiments, r1 is about 19.7 mmol⁻¹ s⁻¹ or greater, calculated based on metal ion concentration.

In some embodiments, r1 is about 2×10⁵ mmol⁻¹ s⁻¹ or greater, calculated based on nanoparticle concentration. In some embodiments, r1 is about 4.9×10⁵ mmol⁻¹ s⁻¹ or greater, calculated based on nanoparticle concentration.

In some embodiments, the contrast agent has a transverse relaxivity (r2) of about 10 mmol⁻¹s⁻¹ or greater, calculated based on metal ion concentration. In some embodiments, r2 is about 60 mmol⁻¹s⁻¹ or greater, calculated based on metal ion concentration. In some embodiments, r2 is about 6.1×10⁵ mmol⁻¹ s⁻¹ or greater, based on nanoparticle concentration. In some embodiments, r2 is about 7.8×10⁵ mmol⁻¹ s⁻¹ or greater, based on nanoparticle concentration.

In some embodiments, the presently disclosed subject matter provides a formulation comprising a hybrid nanoparticle and a pharmaceutically acceptable carrier. In some embodiments, the pharmaceutically acceptable carrier is pharmaceutically acceptable in humans.

In some embodiments, the presently disclosed subject matter provides a method of imaging one of a cell, a tissue, and a subject, the method comprising administering to one of a cell, a tissue, and a subject a contrast agent comprising a hybrid nanoparticle and rendering a magnetic resonance image of the one of a cell, a tissue, and a subject.

In some embodiments, the hybrid nanoparticle further comprises a luminophore. In some embodiments, the method comprises optically imaging the contrast agent.

In some embodiments, the presently disclosed subject matter provides a method of detecting a disease state in one of a cell, a tissue, and a subject.

In some embodiments, the disease state is selected from one of cancer, cardiovascular disease, and a disease associated with inflammation. In some embodiments, the disease state is rheumatoid arthritis.

In some embodiments, the subject is a human.

In some embodiments, the presently disclosed subject matter provides a method of synthesizing a hybrid nanoparticle. In some embodiments, the method comprises synthesizing a hybrid nanoparticle wherein coordination complexes are grafted to the surface of the nanoparticle. In some embodiments, the method comprises synthesizing a layered hybrid nanoparticle.

It is an object of the presently disclosed subject matter to provide hybrid nanoparticles for use as MRI, optical and/or multimodal imaging contrast agents.

An object of the presently disclosed subject matter having been stated hereinabove, and which is achieved in whole or in part by the presently disclosed subject matter, other objects will become evident as the description proceeds when taken in connection with the accompanying drawings and examples as best described hereinbelow.

### BRIEF DESCRIPTION OF THE DRAWINGS

FIG. 1 is a scanning electron microscope (SEM) micrograph of typical silica nanospheres prepared using a water-in-oil microemulsion. The scale bar represents 500 nm.

FIG. 2A is a transmission electron microscope (TEM) micrograph of silica nanoparticles synthesized using a microemulsion having a w-value of 10. The scale bar represents 100 nm.

FIG. 2B is a transmission electron microscope (TEM) micrograph of silica nanoparticles synthesized using a microemulsion having a w-value of 15. The scale bar represents 100 nm.

FIG. 2C is a transmission electron microscope (TEM) micrograph of silica nanoparticles synthesized using a microemulsion having a w-value of 20. The scale bar represents 100 nm.

FIG. 3 is a schematic illustration showing a synthetic route for preparing silica nanoparticles comprising gadolinium-1,4,7,10-tetraazacyclododecane-1,4,7,10-tetraacetic acid (Gd-DOTA)-based chelating groups.

FIG. 4 is a schematic illustration showing a synthetic route for preparing silica nanoparticles comprising gadolinium-bis-aminopropyltrimethoxysilane diethylenetriamine pentaacetate (bis(APS)DTPA-Gd) coordination complex groups.

FIG. 5A is a scanning electron microscope (SEM) micrograph of gadolinium-bis-aminopropyltrimethoxysilane diethylenetriamine pentaacetate (bis(APS)DTPA-Gd)-incorporated silica nanospheres. The distance spanned by all of the scale markings (vertical white lines) represents 1.00 μm, with the distance between each vertical white line representing 100 nm.

FIG. 5B is a scanning electron microscope (SEM) micrograph of gadolinium-bis-aminopropyltrimethoxysilane diethylenetriamine pentaacetate (bis(APS)DTPA-Gd)-incorporated silica nanospheres. The distance spanned by all of the scale markings (vertical white lines) represents 500 nm, with the distance between each vertical white line representing 50 nm.

FIG. 6A is a plot showing a thermogravimetric analysis (TGA) curve of gadolinium-bis-aminopropyltrimethoxysilane diethylenetriamine pentaacetate (bis(APS)DTPA-Gd)-incorporated silica nanospheres having a diameter of approximately 50 nm.

FIG. 6B is a graph showing relaxivity curves for gadolinium-bis-aminopropyltrimethoxysilane diethylenetriamine pentaacetate (bis(APS)DTPA-Gd)-incorporated silica nanospheres having a diameter of approximately 50 nm. The data indicated by the diamonds relates to longitudinal relaxivity (r1), while the data indicated by the triangles relates to the transverse relaxivity (r2).

FIG. 7 is a schematic illustration showing a synthetic route for preparing silica nanoparticles grafted with gadolinium-mono-aminopropyltrimethoxysilane diethylenetriamine tetraacetic acid (mono(APS)DTTA-Gd) coordination complex groups.

FIG. 8A is a scanning electron microscope (SEM) micrograph of Ru(bpy)₃²⁺-doped gadolinium-mono-aminopropyltrimethoxysilane diethylenetriamine tetraacetic acid (mono(APS)DTTA-Gd)-functionalized nanoparticles, 1, prepared from a microemulsion with a w-value of 15. The nanoparticles are spherical, having an average diameter of approximately 37 nm. The distance spanned by all of the scale markings (vertical white lines) represents 500 nm, with the distance between each white vertical line representing 50 nm.

FIG. 8B is a scanning electron microscope (SEM) micrograph of 1, as described for FIG. 8B. The distance spanned by all of the scale markings (vertical white lines) represents 1.00 μm, with the distance between each white vertical line representing 100 nm.

FIG. 9A is a transmission electron microscope (TEM) micrograph showing the 37 nm diameter Ru(bpy)₃²⁺-doped gadolinium-mono-aminopropyltrimethoxysilane diethylenetriamine tetraacetic acid (mono(APS)DTTA-Gd)-functionalized nanoparticles, 1, prepared from a microemulsion with a w-value of 15. The scale bar represents 200 nm.

FIG. 9B is a transmission electron microscope (TEM) micrograph of 40 nm diameter, Ru(bpy)₃²⁺-doped gadolinium-bis-aminopropyltrimethoxysilane diethylenetriamine pentaacetate (bis(APS)DTPA-Gd)-functionalized nanoparticles, 2. The scale bar represents 100 nm.

FIG. 10 is a thermogravimetric analysis (TGA) curve for Ru(bpy)₃²⁺-doped gadolinium-mono-aminopropyltrimethoxysilane diethylenetriamine tetraacetic acid (mono(APS)DTTA-Gd)-functionalized nanoparticles, 1, prepared from a microemulsion with a w-value of 15 and having a diameter of approximately 37 nm.

FIG. 11 is a graph of absorbance spectra of aqueous Ru(bpy)₃²⁺ (upper dashed line) and of Ru(bpy)₃²⁺-doped gadolinium-mono-aminopropyltrimethoxysilane diethylenetriamine tetraacetic acid (mono(APS)DTTA-Gd)-functionalized nanoparticles, 1, prepared from a microemulsion with a w-value of 15 and having an average diameter of approximately 37 nm (lower dashed line). The graph also shows emission spectra of aqueous Ru(bpy)₃²⁺ (lower solid line) and of 1 (upper solid line). An excitation wavelength of 488 nm was used to collect the emission spectra.

FIG. 12 is a graph showing relaxivity curves for Ru(bpy)₃²⁺-doped gadolinium-mono-aminopropyltrimethoxysilane diethylenetriamine tetraacetic acid (mono(APS)DTTA-Gd)-functionalized nanoparticles, 1, prepared from a microemulsion with a w-value of 15 and having an average diameter of approximately 37 nm. The data indicated by the squares relates to longitudinal relaxivity (r1), while the data indicated by the diamonds relates to the transverse relaxivity (r2).

FIG. 13 is a scanning electron microscope (SEM) micrograph of Ru(bpy)₃²⁺-doped gadolinium-mono-aminopropyltrimethoxysilane diethylene-triamine tetraacetic acid (mono(APS)DTTA-Gd)-functionalized nanoparticles having a average diameter of approximately 45 nm. The distance spanned by all of the scale markings (vertical white lines) represents 1.00 μm, with the distance between each white vertical line representing 100 nm.

FIG. 14 is a plot showing a thermogravimetric analysis (TGA) curve of 40 nm diameter, Ru(bpy)₃²⁺-doped gadolinium-bis-aminopropyltrimethoxysilane diethylenetriamine pentaacetate (bis(APS)DTPA-Gd)-incorporated silica nanospheres, 2.

FIG. 15 is a schematic drawing highlighting structural differences between 1 (Ru(bpy)₃²⁺-doped gadolinium-mono-aminopropyltrimethoxysilane diethylenetriamine tetraacetic acid (mono(APS)DTTA-Gd)-functionalized nanoparticles made by grafting mono(APS)-DTTA-Gd chelating groups on the surface of silica nanoparticles) and 2 (Ru(bpy)₃²⁺-doped gadolinium-bis-aminopropyltrimethoxysilane diethylenetriamine pentaacetate (bis(APS)DTPA-Gd)-functionalized silica nanoparticles made with polymerizable bis(APS)DTPA groups). The bis(APS)-derivatized chelating group used in the synthesis of 2 is capable of forming a polymeric layer over the surface of the nanoparticle.

FIG. 16 is a composite image of T1-weighted (left) and T2-weighted (right) phantom magnetic resonance (MR) images of silica nanoparticles (SNPs) 1 (top row) and 2 (middle row) dispersed in water at concentrations of 0.30, 0.15, and 0.05 mM. Images of OMNISCAN™ (GE Healthcare, Princeton, N.J., United States of America) (bottom row) at the same concentrations are included for comparison.

FIG. 17 is a scanning electron microscope (SEM) micrograph of Ru(bpy)₃²⁺-doped gadolinium-bis-aminopropyltrimethoxysilane diethylene-triamine pentaacetate (bis(APS)DTPA-Gd)-functionalized nanoparticles having a diameter of approximately 50 nm. The distance spanned by all of the scale markings (vertical white line) represents 1.00 μm, with the distance between each white vertical line representing 100 nm.

FIG. 18 is a scanning electron microscope (SEM) micrograph of typical polyethylene glycol (PEG)- and fluorescein isothiocyanate (FITC)-grafted silica nanospheres prepared according to the methods of the presently disclosed subject matter. The distance spanned by all of the scale markings (vertical white lines) represents 500 nm, with the distance between each vertical white line representing 50 nm.

FIG. 19 is a schematic illustration showing a synthetic route for the preparation of hybrid nanomaterials according to a layer-by-layer deposition technique. The dark colored circle represents the polymeric matrix material forming the core of a nanoparticle (optionally grafted to coordination complexes). The grey layers represent layers of positively charged polymerized coordination complexes, poly[Gd-chelate)⁺]. The striped layer represents an anionic layer comprising poly(styrene sulfonate) (PSS).

FIG. 20A is a graph showing relaxivity curves for silica nanoparticles comprising surface grafted gadolinium-mono-aminopropyltrimethoxysilane diethylenetriamine tetraacetic acid (mono(APS)DTTA-Gd) coordination complex groups. The data indicated by the diamonds was used to calculate longitudinal relaxivity (r1), while the data indicated by the triangles was used to calculate transverse relaxivity (r2).

FIG. 20B is a graph showing relaxivity curves for silica nanoparticles of sample 3, three layer nanoparticles which comprise the nanoparticles described for FIG. 20A, further comprising a positively charged poly[(Gd chelate)⁺] layer and an anionic poly(styrene sulfonate) (PSS) layer. The data indicated by the diamonds was used to calculate longitudinal relaxivity (r1), while the data indicated by the triangles was used to calculate transverse relaxivity (r2).

FIG. 20C is a graph showing relaxivity curves for silica nanoparticles of sample 4, the nanoparticles described for FIG. 20B, further comprising an additional poly[(Gd chelate)⁺] layer and an additional poly(styrene sulfonate) (PSS) layer. The data indicated by the diamonds was used to calculate longitudinal relaxivity (r1), while the data indicated by the triangles was used to calculate transverse relaxivity (r2).

FIG. 20D is a graph showing relaxivity curves for silica nanoparticles of sample 5, the nanoparticles described for FIG. 20C, further comprising an additional poly[(Gd chelate)⁺] layer and an additional poly(styrene sulfonate) (PSS) layer. The data indicated by the diamonds was used to calculate longitudinal relaxivity (r1), while the data indicated by the triangles was used to calculate transverse relaxivity (r2).

FIG. 21 is a schematic illustration showing a synthetic route for the preparation of nanomaterials comprising poly(acrylic acid).

FIG. 22A is a schematic drawing showing a synthetic route for the preparation of nanoparticles comprising a mono-functionalized gadolinium-aminopropyltrimethoxysilane diethylenetriamine pentaacetate (DTPA-Gd) coordination complex group comprising a single biodegradable linkage.

FIG. 22B is a schematic drawing showing a synthetic route for the preparation of nanoparticles comprising a polymerizable gadolinium-aminopropyltrimethoxysilane diethylenetriamine pentaacetate (DTPA-Gd) coordination complex group comprising a biodegradable linkage in each of the groups linking a reactive siloxy group to the DTPA chelator.

FIG. 23A is an optical microscopic image of the cellular uptake of polyethylene glycol (PEG) and aminopropyl trimethoxysilane-functionalized fluorescein (APS-FITC) coated silica nanoparticles by monocyte cells.

FIG. 23B is a fluorescence microscope image of the cellular uptake of polyethylene glycol (PEG) and aminopropyl trimethoxysilane-functionalized fluorescein (APS-FITC) coated silica nanoparticles by monocyte cells.

FIG. 23C is an optical microscopic image of the cellular uptake of polyethylene glycol (PEG) and aminopropyl trimethoxysilane-functionalized fluorescein (APS-FITC) coated silica nanoparticles by HeLa S3 cells.

FIG. 23D is a fluorescence microscope image of the cellular uptake of polyethylene glycol (PEG) and aminopropyl trimethoxysilane-functionalized fluorescein (APS-FITC) coated silica nanoparticles by HeLa S3 cells.

FIG. 24A is an optical microscope image of monocyte cellular uptake of Ru(bpy)₃²⁺-imbedded gadolinium-mono-aminopropyltrimethoxysilane diethylenetriamine tetraacetic acid (mono(APS)DTTA-Gd)-functionalized silica particles.

FIG. 24B is a confocal laser scanning fluorescence image of monocyte cellular uptake of Ru(bpy)₃²⁺-imbedded gadolinium-mono-aminopropyltrimethoxysilane diethylenetriamine tetraacetic acid (mono(APS)DTTA-Gd)-functionalized silica particles. The scale bar represents 12 μm.

FIG. 25A is a confocal laser scanning fluorescence image of a frozen slice of inflamed mouse intestine that is labeled with Ru(bpy)₃²⁺-imbedded gadolinium-mono-aminopropyltrimethoxysilane diethylenetriamine tetraacetic acid (mono(APS)DTTA-Gd)-functionalized silica nanoparticles which further comprise an anti-major histocompatibility complex (MHC)-II antibody as a targeting agent.

FIG. 25B is a confocal laser scanning fluorescence image of a frozen slice of inflamed mouse intestine that is labeled with Ru(bpy)₃²⁺-imbedded gadolinium-mono-aminopropyltrimethoxysilane diethylenetriamine tetraacetic acid (mono(APS)DTTA-Gd)-functionalized silica nanoparticles which comprise anti-MHC-II antibody as a targeting agent.

FIG. 26A is a microscopic image of monocyte cells labeled with 1 (37 nm diameter, Ru(bpy)₃²⁺-doped gadolinium-mono-aminopropyltrimethoxysilane diethylenetriamine tetraacetic acid (mono(APS)DTTA-Gd)-functionalized nanoparticles prepared from a microemulsion with a w-value of 15). To prepare the labeled cells, monocyte cells (1×10⁶) were incubated with 0.42 mg of 1 for 30 minutes.

FIG. 26B is a laser scanning confocal fluorescence microscopic image of the 1-labeled monocyte cells described for FIG. 26A. Ligand-to-metal charge transfer (LMCT) luminescence from the Ru(bpy)₃²⁺ can be detected.

FIG. 26C is a T1-weighted magnetic resonance (MR) image of the 1-labeled monocyte cells described for FIG. 26A.

FIG. 26D is a T2-weighted magnetic resonance (MR) image of the 1-labeled monocyte cells described for FIG. 26A.

FIG. 26E is a graph showing the flow cytometric results of the labeling efficiency of monocyte cells (1×10⁶ cells) with 1 (0.42 mg). The peak on the left is for the unlabeled monocyte cells, prior to exposure to 1. The peak on the right is for the 1-labeled monocytes cells. The results indicate a greater than 98% labeling efficiency. The inset shows the purity of the labeled cells. SS and FS refer to side-scattering and forward-scattering signals, respectively.

FIG. 26F is a bar graph of the results of the 3-(4,5-dimethylthiazol-2-yl)-5-(3-carboxy-methoxyphenyl)-2-(4-sulfophenyl)-2H-tetrazolium (MTS) toxicity assay of monocyte cells (5000 cells) incubated with different amounts (i.e., 0, 0.012, 0.123, 1.23, 12.3, or 123 μg, respectively, from left to right) of 1.

FIG. 27A is a pre-contrast MR image of a choroids plexus carcinoma (CPC) mouse model.

FIG. 27B is an MR image of the CPC mouse model immediately after tail vein injection of 25 mg of hybrid nanoparticles.

FIG. 27C is an MR image taken 5 hours after the injection of hybrid nanoparticles.

FIG. 28A is a confocal microscopic optical (right) and fluorescence (left) image of HT-29 colon cancer cells without any nanoparticle.

FIG. 28B is a confocal microscopic optical (right) and fluorescence (left) image of HT-29 colon cancer cells after incubation with RGD-targeted layer-by-layer (LBL) nanoparticles.

FIG. 28C is a confocal microscopic optical (right) and fluorescence (left) image of the HT-29 colon cancer cells after being incubated with LBL nanoparticles that are terminated with a PSS layer.

FIG. 28D is a confocal microscopic optical (right) and fluorescence (left) image of the HT-29 colon cancer cells after being incubated with GRD-targeted LBL nanoparticles.

FIG. 29 is a T1-weighted MR image of pellets of HT-29 cells with the following treatments (from left to right, as indicated by the arrows): no incubation with nanoparticles, after incubation with LBL nanoparticles that are terminated with a PSS layer, after incubation with RGD-targeted LBL nanoparticles, and after incubation with GRD-targeted LBL nanoparticles.

### DETAILED DESCRIPTION

The presently disclosed subject matter will now be described more fully hereinafter with reference to the accompanying Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

All publications, patent applications, patents, and other references mentioned herein are incorporated by reference in their entirety.

Throughout the specification and claims, a given chemical formula or name shall encompass all optical and stereoisomers, as well as racemic mixtures where such isomers and mixtures exist.

### I. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in this application, including the claims. Thus, for example, reference to “a metal ion” includes a plurality of such metal ions, and so forth.

Unless otherwise indicated, all numbers expressing quantities of size, MRI relaxivity, number of metal ions, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about”, when referring to a value or to an amount of size (i.e., diameter), weight, concentration or percentage is meant to encompass variations of in one example ±20% or ±10%, in another example ±5%, in another example ±1%, and in still another example ±0.1% from the specified amount, as such variations are appropriate to perform the disclosed methods.

The terms “nanomaterial” and “nanoparticle” refer to a structure having at least one region with a dimension (e.g., length, width, diameter, etc.) of less than about 1,000 nm. In some embodiments, the dimension is smaller (e.g., less than about 500 nm, less than about 250 nm, less than about 200 nm, less than about 150 nm, less than about 125 nm, less than about 100 nm, less than about 80 nm, less than about 70 nm, less than about 60 nm, less than about 50 nm, less than about 40 nm, less than about 30 nm or even less than about 20 nm). In some embodiments, the dimension is less than about 10 nm.

In some embodiments, the nanomaterial or nanoparticle is approximately spherical. When the nanoparticle is approximately spherical, the characteristic dimension can correspond to the diameter of the sphere (i.e. is a nanosphere). In addition to spherical shapes, the nanomaterial can be disc-shaped, oblong, polyhedral, rod-shaped, cubic, or irregularly-shaped.

The nanoparticle can comprise a core region (i.e., the space between the outer dimensions of the particle) and an outer surface (i.e., the surface that defines the outer dimensions of the particle). In some embodiments, the particle can comprise one or more layers. Thus, for example, a spherical nanoparticle can comprise one or more concentric layers, each successive layer being dispersed over the outer surface of smaller layer closer to the center of the particle. The particle can be solid or porous or can contain a hollow interior region. Typically, the core or one or more layer of the nanoparticles described herein can comprise a polymeric matrix material, but can also comprise one or more coordination complexes, optical imaging agents or other groups.

When the core comprises coordination complexes or optical imaging agents, the complexes or agents can be said to be “embedded” in the nanoparticle. “Embedded” can refer a coordination complex or an optical imaging agent that is bound, for example covalently bound, inside the core of the particle (e.g., to the polymeric matrix material or to another coordination complex or optical imaging agent) or to a coordination complex or optical imaging agent (such as a semiconducting CdSe quantum dot or a Mn-doped CdSe quantum dot) that is non-covalently associated with the core of the nanoparticle. For, example, the complex or agent can be sequestered (i.e., non-covalently encapsulated) inside pores in the polymeric matrix material or can interact with the polymeric matrix material via hydrogen bonding, London dispersion forces, or any other non-covalent interaction.

The terms “polymer” and “polymeric” refer to chemical structures that have repeating units (i.e., multiple copies of a given chemical substructure). Polymers can be formed from polymerizable monomers. A polymerizable monomer is a molecule that comprises one or more reactive moieties that can react to form covalent bonds with reactive moieties on other molecules of polymerizable monomer. Generally, each polymerizable monomer molecule can bond to two or more other molecules. In some cases, a polymerizable monomer will bond to only one other molecule, forming a terminus of the polymeric material.

Polymers can be organic, or inorganic, or a combination thereof. As used herein, the term “inorganic” refers to a compound or composition that contains at least some atoms other than carbon, hydrogen, nitrogen, oxygen, sulfur, phosphorous, or one of the halides. Thus, for example, an inorganic compound or composition can contain one or more silicon atoms.

The term “contrast agent” refers to a moiety (a specific part of or an entire molecule, macromolecule, coordination complex, or nanoparticle) that increases the contrast of a tissue or structure being examined. The contrast agent can increase the contrast of a structure being examined using magnetic resonance imaging (MRI), optical imaging, or a combination thereof (i.e., the contrast agent can be multimodal).

The term “MRI contrast agent” refers to a moiety that effects a change in induced relaxation rates of water protons in a sample.

The terms “optical imaging agent” or “optical contrast agent” refer to a group that can be detected based upon an ability to absorb, reflect or emit light (e.g., ultraviolet, visible, or infrared light). Optical imaging agents can be detected based on a change in amount of absorbance, reflectance, or fluorescence, or a change in the number of absorbance peaks or their wavelength maxima. Thus, optical imaging agents include those which can be detected based on fluorescence or luminescence, including organic and inorganic dyes.

As used herein, the term “ligand” refers generally to a chemical species, such as a molecule or ion, which interacts (e.g., binds) in some way with another species. The term “ligand” can refer to a molecule or ion that binds a metal ion in solution to form a “coordination complex.” See Martell, A. E., and Hancock, R. D., Metal Complexes in Aqueous Solutions, Plenum: N.Y. (1996), which is incorporated herein by reference in its entirety. The term “ligand” can also refer to a molecule involved in a biospecific recognition event (e.g., antibody-antigen binding, enzyme-substrate recognition, receptor-receptor ligand binding, etc).

A “coordination complex” is a compound in which there is a coordinate bond between a metal ion and an electron pair donor (i.e., chelating group). Thus, chelating groups are generally electron pair donors, molecules or molecular ions having unshared electron pairs available for donation to a metal ion.

The terms “bonding” or “bonded” and variations thereof can refer to either covalent or non-covalent bonding. In some cases, the term “bonding” refers to bonding via a coordinate bond. The term “conjugation” can refer to a bonding process, as well, such as the formation of a covalent linkage or a coordinate bond.

The term “coordination” refers to an interaction in which one multi-electron pair donor coordinately bonds, i.e., is “coordinated,” to one metal ion.

The term “coordinate bond” refers to an interaction between an electron pair donor and a coordination site on a metal ion resulting in an attractive force between the electron pair donor and the metal ion. The use of this term is not intended to be limiting, in so much as certain coordinate bonds also can be classified as have more or less covalent character (if not entirely covalent character) depending on the characteristics of the metal ion and the electron pair donor.

The term “coordination site” refers to a point on a metal ion that can accept an electron pair donated, for example, by a chelating agent.

The terms “chelating agent,” “metal coordination ligand,” “chelating group,” and “chelator” refer to a molecule or molecular ion or species having an unshared electron pair available for donation to a metal ion. In some embodiments, the metal ion is coordinated by two or more electron pairs to the chelating agent. The terms “bidentate chelating agent,” “tridentate chelating agent,” “tetradentate chelating agent,” and “pentadentate chelating agent” refer to chelating agents having two, three, four, and five electron pairs, respectively, available for simultaneous donation to a metal ion coordinated by the chelating agent. In some embodiments, the electron pairs of a chelating agent form coordinate bonds with a single metal ion. In some embodiments, the electron pairs of a chelating agent form coordinate bonds with more than one metal ion, with a variety of binding modes being possible.

As used herein, the term “paramagnetic metal ion” refers to a metal ion that is magnetized parallel or antiparallel to a magnetic field to an extent proportional to the field. Generally, paramagnetic metal ions are metal ions that have unpaired electrons. Paramagnetic metal ions can be selected from the group consisting of transition and inner transition elements, including, but not limited to, scandium, titanium, vanadium, chromium, cobalt, nickel, copper, molybdenum, ruthenium, cerium, praseodymium, neodymium, promethium, samarium, europium, terbium, holmium, erbium, thulium, and ytterbium. In some embodiments, the paramagnetic metal ions can be selected from the group consisting of gadolinium III (i.e., Gd⁺³ or Gd(III)); manganese II (i.e., Mn⁺² or Mn(II)); copper II (i.e., Cu⁺² or Cu(II)); chromium III (i.e., Cr⁺³ or Cr(II)); iron II (i.e., Fe⁺² or Fe(II)); iron III (i.e., Fe⁺³ or Fe(III)); cobalt II (i.e., Co⁺² or Co(II)); erbium II (i.e., Er⁺² or Er(II)), nickel II (i.e., Ni⁺² or Ni(II)); europium III (i.e., Eu⁺³ or Eu(III)); yttrium III (i.e., Yt⁺³ or Yt(III)); and dysprosium III (i.e., Dy⁺³ or Dy(III)). In some embodiments, the paramagnetic ion is the lanthanide atom Gd(III), due to its high magnetic moment, symmetric electronic ground state, and its current approval for diagnostic use in humans.

The term “functionalized chelating group” refers to a species that includes a chelator (i.e., a metal coordination ligand), as well as groups that can conjugate (i.e., via covalent or non-covalent bonds) the chelator or chelator metal complex to another chemical species. In some embodiments, the functionalized chelating group includes groups that can covalently bond to another chemical species, such as to a polymeric matrix material, one or more other functionalized chelating groups, or to additional groups, such as targeting agents, circulation enhancing groups, optical imaging agents, and the like. Thus, a “functionalized chelating group” can include one or more reactive moieties, chemical species that can react with other chemical groups to form covalent bonds. Reactive moieties can include, but are not limited to siloxy ethers, vinylic groups (i.e., carbon-carbon double bonds), halides, esters, activated esters, and the like.

In some embodiments, the polymeric matrix material or the functionalized chelating group includes a degradable linkage (i.e., a chemical bond that is designed to break or cleave during the delivery or use of the contrast enhancement agent). For example, the functionalized chelating group can comprise a degradable linkage designed to break so that the chelating group can become free of the nanoparticle. Cleavage can involve hydrolysis, reduction, or any type of homolytic or heterolytic bond cleavage.

In some embodiments, the degradable linkage is a biodegradable linkage. The term “biodegradable linkage” refers to a linkage that breaks in response to a biological stimulus, such as an enzyme or to a given physiological condition, such as a particular pH. The biological stimulus can be related to a specific tissue or to a specific disease. The stimulus can be related to pH changes that occur upon phagocytosis (or another type of uptake) of a nanoparticle by a cell. Biodegradable linkages include, but are not limited to amides, carbamates (including aryl carbamates), esters, and disulfide bonds.

The term “copolymer” refers to a polymer formed from two or more different (i.e., not having the same chemical formula) polymerizable monomers. Structures resulting from the different polymerizable monomers can be mixed throughout the final copolymer. Alternatively, the majority of each polymerizable monomer can react with other monomers of the same chemical formula, and the resulting copolymer will comprise blocks of oligomers of the different monomers. Such a structure can be referred to as a “block copolymer.”

“Luminescence” occurs when a molecule (or other chemical species) in an electronically excited state relaxes to a lower energy state by the emission of a photon. The luminescent agent in one embodiment can be a chemiluminescent agent. In chemiluminescence, the excited state is generated as a result of a chemical reaction, such as lumisol and isoluminol. In photoluminescence, such as fluorescence and phosphorescence, an electronically excited state is generated by the illumination of a molecule with an external light source. Bioluminescence can occur as the result of action by an enzyme, such as luciferase. In electrochemiluminescence (ECL), the electronically excited state is generated upon exposure of the molecule (or a precursor molecule) to electrochemical energy in an appropriate surrounding chemical environment. Examples of electrochemiluminescent agents are provided, for example, in U.S. Pat. Nos. 5,147,806; and 5,641,623; and in U.S. Patent Application Publication No. 2001/0018187; and include, but are not limited to, metal cation-liquid complexes, substituted or unsubstituted polyaromatic molecules, and mixed systems such as aryl derivatives of isobenzofurans and indoles. The electrochemiluminescent chemical moiety can comprise, in a specific embodiment, a metal-containing organic compound wherein the metal is selected from the group consisting of ruthenium, osmium, rhenium, iridium, rhodium, platinum, palladium, molybdenum, technetium and tungsten.

As described above, the term “fluorophore” refers to a species that can be excited by visible light or non-visible light (e.g., UV light). Examples of fluorophores include, but are not limited to: quantum dots and doped quantum dots (e.g., a semiconducting CdSe quantum dot or a Mn-doped CdSe quantum dot), fluorescein, fluorescein derivatives and analogues, indocyanine green, rhodamine, triphenylmethines, polymethines, cyanines, phalocyanines, naphthocyanines, merocyanines, lanthanide complexes or cryptates, fullerenes, oxatellurazoles, LaJolla blue, porphyrins and porphyrin analogues and natural chromophores/fluorophores such as chlorophyll, carotenoids, flavonoids, bilins, phytochrome, phycobilins, phycoerythrin, phycocyanines, retinoic acid and analogues such as retinoins and retinates.

The term “quantum dot” refers to semiconductor nanoparticles comprising an inorganic crystalline material that is luminescent (i.e., that is capable of emitting electromagnetic radiation upon excitation). The quantum dot can include an inner core of one or more first semiconductor materials that is optionally contained within an overcoating or “shell” of a second semiconductor material. A semiconductor nanocrystal core surrounded by a semiconductor shell is referred to as a “core/shell” semiconductor nanocrystal. The surrounding shell material can optionally have a bandgap energy that is larger than the bandgap energy of the core material and can be chosen to have an atomic spacing close to that of the core substrate.

Suitable semiconductor materials for quantum dots include, but are not limited to, materials comprising a first element selected from Groups 2 and 12 of the Periodic Table of the Elements and a second element selected from Group 16. Such materials include, but are not limited to ZnS, ZnSe, ZnTe, CDs, CdSe, CdTe, HgS, HgSe, HgTe, MgS, MgSe, MgTe, CaS, CaSe, CaTe, SrS, SrSe, SrTe, BaS, BaSe, BaTe, and the like. Suitable semiconductor materials also include materials comprising a first element selected from Group 13 of the Periodic Table of the Elements and a second element selected from Group 15. Such materials include, but are not limited to, GaN, GaP, GaAs, GaSb, InN, InP, InAs, InSb, and the like. Semiconductor materials further include materials comprising a Group 14 element (Ge, Si, and the like); materials such as PbS, PbSe and the like; and alloys and mixtures thereof. As used herein, all reference to the Periodic Table of the Elements and groups thereof is to the new IUPAC system for numbering element groups, as set forth in the Handbook of Chemistry and Physics, 81st Edition (CRC Press, 2000).

As used herein the term “alkyl” refers to C₁₋₂₀ inclusive, linear (i.e., “straight-chain”), branched, or cyclic, saturated or at least partially and in some cases fully unsaturated (i.e., alkenyl and alkynyl)hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds. The aromatic ring(s) can comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Specific examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

In some embodiments, the compounds described by the presently disclosed subject matter contain a linking group. As used herein, the term “linking group” comprises a chemical moiety, such as a alkylene, furanyl, phenylene, thienyl, and pyrrolyl radical, which is bonded to two or more other chemical moieties to form a stable structure.

“Alkylene” refers to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons.

As used herein, the term “acyl” refers to an organic carboxylic acid group wherein the —OH of the carboxyl group has been replaced with another substituent (i.e., as represented by RCO—, wherein R is an alkyl or an aryl group as defined herein). As such, the term “acyl” specifically includes arylacyl groups, such as an acetylfuran and a phenacyl group. Specific examples of acyl groups include acetyl and benzoyl.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl.

“Alkoxyl” refers to an alkyl-O— group wherein alkyl is as previously described. The term “alkoxyl” as used herein can refer to, for example, methoxyl, ethoxyl, propoxyl, isopropoxyl, butoxyl, t-butoxyl, and pentoxyl. The term “oxyalkyl” can be used interchangably with “alkoxyl”.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl-group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl.

“Dialkylamino” refers to an —NRR′ group wherein each of R and R′ is independently an alkyl group and/or a substituted alkyl group as previously described. Exemplary dialkylamino groups include ethylmethylamino, dimethylamino, and diethylamino. “Alkylamino” refers to a —NRR′ group wherein one of R and R′ is H and the other of R and R′ is alkyl.

“Alkoxycarbonyl” refers to an alkyl-O—CO— group. Exemplary alkoxycarbonyl groups include methoxycarbonyl, ethoxycarbonyl, butyloxycarbonyl, and t-butyloxycarbonyl.

“Aryloxycarbonyl” refers to an aryl-O—CO— group. Exemplary aryloxycarbonyl groups include phenoxy- and naphthoxy-carbonyl.

“Aralkoxycarbonyl” refers to an aralkyl-O—CO— group. An exemplary aralkoxycarbonyl group is benzyloxycarbonyl.

“Carbamoyl” refers to an H₂N—CO— group.

“Alkylcarbamoyl” refers to a R′RN—CO— group wherein one of R and R′ is hydrogen and the other of R and R′ is alkyl and/or substituted alkyl as previously described.

“Dialkylcarbamoyl” refers to a R′RN—CO— group wherein each of R and R′ is independently alkyl and/or substituted alkyl as previously described.

“Acyloxyl” refers to an acyl-O— group wherein acyl is as previously described.

“Acylamino” refers to an acyl-NH— group wherein acyl is as previously described.

The term “amino” refers to the —NH₂ group. “Amino” can also refer to a dialkylamino or alkylamino group as described above.

The term “carbonyl” refers to the —(C═O)— group.

The terms “carboxylate,” “carboxylic acid,” “acetic acid” and “acetate” refer to the —C(═O)OH or —C(═O)O⁻ group. As will be understood by one of skill in the art, the protonation state of the group will vary according to the chemical environment. Thus, the terms “acetate” and “acetic acid” can be used interchagably.

The term “ester” refers to the —C(═O)OR group, wherein R can be alkyl, substituted alkyl, cycloalkyl, aryl, substituted aryl, aralkyl, and the like. Thus, the term “ester” can be used to refer to molecules containing alkoxycarbonyl, aryloxycarbonyl, and aralkoxycarbonyl groups.

The term “amide” refers to molecules containing a —NR—C(═O)— group,

wherein R is H, alkyl, aralkyl, or aryl. Thus, an amide can include an acylamino, carbamoyl, alkylcarbamoyl or dialkylcarbamoyl group as defined above.

The term “carbamate” refers to the R—NH—C(═O)—O—R′ group, wherein R and R′ are alkyl, substituted alkyl, aryl, substituted aryl, or aralkyl. In an aryl carbamate, the R′ group is aryl or substituted aryl.

The terms “halo”, “halide”, or “halogen” as used herein refer to fluoro, chloro, bromo, and iodo groups.

The term “hydroxyl” refers to the —OH group.

The term “hydroxyalkyl” refers to an alkyl group substituted with an —OH group.

The terms “mercapto” or “thiol” refer to the —SH group.

The term “oxo” refers to a compound described previously herein wherein a carbon atom is replaced by an oxygen atom.

The term “nitro” refers to the —NO₂ group.

The term “thio” refers to a compound described previously herein wherein a carbon or oxygen atom is replaced by a sulfur atom.

The term “sulfate” refers to the —SO₄⁻ group.

The term “phosphonate” refers to the —P(═O)(OR)₂ group, wherein R can be H, alkyl, aralkyl, aryl, or a negative charge.

The term “silyl” refers to groups comprising silicon atoms (Si).

As used herein, the terms “siloxy” and “silyl ether” refer to groups or compounds including a silicon-oxygen (Si—OR) bond. In some embodiments, the terms refer to compounds comprising one, two, three, or four alkoxy, aralkoxy, or aryloxy groups bonded to a silicon atom. Each alkyloxy, aralkoxy, or aryloxy group can be the same or different.

The term “silanol” refers to the Si—OH group.

The term “siloxane” refers to a compound comprising a —Si—O—Si-linkage.

The term “hydrophilic” refers to the ability of a molecule or chemical species to interact with water. Thus, hydrophilic molecules are typically polar or have groups that can hydrogen bond to water. The term “hydrophobic” refers to a molecule that interacts poorly with water (e.g., does not dissolve in water or does not dissolve in water to a large extent).

The term “lipophilic” refers to a molecule or chemical species that interacts (e.g., dissolves in) fat or lipids.

The term “amphiphilic” refers to a molecule or species that has both hydrophilic and hydrophobic (or lipophilic) attributes.

### II. Hybrid Nanoparticles

Descriptions of luminescent nanoparticles in biological and biomedical imaging (see Alivisatos et al., Nat. Biotechnol., 22, 47 (2004); Kim et al., J. Am. Chem. Soc., 127, 10526 (2005); Gao et al., Nat. Biotechnol. 22, 969 (2004), Giepmans et al., Science, 312, 217 (2006); and Sandros et al., J. Am. Chem. Soc., 127, 12198-12199 (2005)) have brought to light a need for new nanomaterials for use in other imaging techniques, particularly MRI. See Cheng et al., Curr. Opin. Chem. Biol., 10, 11 (2006). In the development of new MRI contrast agents, extensive studies have shown r1 enhancement for simple molecular metal chelates can be accomplished through a variety of approaches, most important of which include (1) increasing the rotational correlation time of the molecule by increasing its molecular weight or size, and (2) by increasing the inner sphere water coordination number of the metal chelate to a high molecular weight moiety such as a synthetic polymer or naturally occurring protein. Recent work has demonstrated that iron oxide nanoparticles can be used as target-specific MR contrast agents for tumor angiogenesis, inflammation, and gene expression. See Weissleder et al., Nat. Med., 6, 351 (2000); and Song et al., J. Am. Chem. Soc., 127, 9992-9993 (2005). Other recent work has shown that Gd³⁺ microemulsions can provide a platform for designing nanoscale T1 contrast agents. See Morawski et al., Curr. Opin. Biotechnol., 16, 89 (2005). For example, up to 50,000 Gd³⁺ centers can be loaded into a liposome several hundred nanometers in diameter which can then be molecularly targeted to a variety of biomarkers that are specifically overexpressed in diseased states, such as tumors and coronary artery diseases. See Mulder et al., NMR Biomed., 19, 142 (2006).

The presently disclosed subject matter provides nanoparticles that contain a large number of chelated paramagnetic metal ions, and are thus able to exhibit a large r1 relaxivity on a per nanoparticle basis. The nanoparticles can also be easily functionalized with optical imaging agents, targeting agents, and other groups. Thus, the presently disclosed nanoparticles provide a highly useful platform for the design and preparation of smart, target-specific, multimodal imaging contrast agents that can be used for early cancer detection or inflammation imaging, among other uses.

Accordingly, the presently disclosed subject matter provides a hybrid nanoparticle for use as a magnetic resonance imaging contrast agent. The hybrid nanoparticles of the presently disclosed subject matter can comprise a polymeric matrix material and a plurality of coordination complexes, wherein each coordination complex comprises a functionalized chelating group and a paramagnetic metal ion.

In some embodiments, the presently disclosed hybrid nanoparticles comprise a multimodal imaging agent (i.e., an imaging agent that can be detected via more than one imaging technique). Thus, in some embodiments, the hybrid nanoparticle comprises an optically detectable moiety in addition to the paramagnetic metal ions which allow for detection via magnetic resonance imaging. In some embodiments, the additional detectable moiety is a luminophore. The luminophore can be either organic or inorganic. In some embodiments, the luminophore is a fluorophore. In some embodiment, the fluorophore is selected from the group consisting of ruthenium(II) tris(2,2′bipyridine) (i.e., Ru(bpy)₃²⁺) and fluoroscein isothiocyanate (FITC).

The luminophore or fluorophore can be imbedded in the hybrid nanoparticle. Thus, the luminophore or fluorophore can be dispersed throughout the polymeric matrix material, and can be covalently bound to the polymeric matrix material or simply sequestered (non-covalently) in pores present in the polymeric matrix. In some embodiments, the luminophore or fluorophore is bonded to an outer surface of the nanoparticle. The bond between the luminophore or fluorophore and the nanoparticle surface can comprise a covalent bond, for example, between a reactive group on the luminophore and the polymeric matrix material. The luminophore or fluorophore can also be bonded to a reactive moiety on a functionalized chelating group. When a group is bonded to the outer surface of a nanoparticle, it can also be referred to as being “grafted” to the surface of the nanoparticle.

The polymeric matrix material can be either an organic (i.e., carbon-based) or an inorganic (i.e., non-carbon-based) material. Alternatively, the polymeric matrix material can comprise both inorganic and organic components. For example, the polymeric matrix can comprise a copolymer of inorganic and organic monomers. In some embodiments, the polymeric matrix material can comprise a copolymer of different organic monomers or a copolymer of different inorganic monomers.

In some embodiments, the polymeric matrix material is an inorganic polymer. In some embodiments, the inorganic polymer comprises silicon. In some embodiments, the inorganic polymer is a siloxane or SiO₂. The inorganic polymer can be formed, for example, from the polycondensation of silyl ethers. In some embodiments, the inorganic polymer can be formed from the polymerization of tetraethyl orthosilicate (TEOS; i.e., Si(OCH₂CH₃)₄). The polymerization of TEOS involves two types of chemical reactions: a hydrolysis reaction in which one or more ethoxy group is hydrolyzed to form a silanol group (e.g., Si(OCH₂CH₃)₃(OH)); followed by a condensation reaction wherein two silanols (i.e., silanol groups on two different molecules) or a silanol and a silyl ether group (again on different molecules) react (i.e., condense) to form a siloxane bond (i.e., Si—O—Si) and a molecule of either water or ethanol. In some embodiments, the polymer comprises only siloxane linkages. In some embodiments, some of the ethoxy groups remain. The extent of polymerization can be controlled to tailor the hydrophobicity or pore size of the matrix material.

In some embodiments, the polymeric matrix comprises an organic polymer. Suitable organic polymers include, but are not limited to, polyolefins, polyesters, polyamides, polyethers, and combinations thereof. In some embodiments, the organic polymer can be prepared from an acrylate monomer (i.e., a compound comprising the group CH₂═CH—C(═O)—), such as, acrylic acid, butyl acrylate, methyl acrylate, ethyl acrylate, acrylonitrile, methyl methacrylate, or trimethylol propane triacrylate (TMPTA). In some embodiments, the organic polymer is selected from the group consisting of polyacrylic acid and polylactide (PLA; i.e., [—O—CH(CH₃)—C(═O)-]ₙ).

In some embodiments, the polymeric matrix material is biodegradable. For example, the polymeric matrix material can comprises linkages that degrade under physiological conditions, such as the presence of a pH associated with a specific biological environment or in the presence of a particular enzyme. The enzyme can be associated with a general biological environment, such as blood or plasma, or can be an enzyme or physiological condition associated with a particular disease state, such as a cancer. One example of a biodegradable polymeric matrix material is PLA, which comprises multiple hydrolyzable ester bonds. Thus, in some embodiments, the hybrid nanoparticle is designed to degrade in the biological environment, for example in a living subject (e.g., a human patient), over time, allowing for the programmed clearance (i.e., elimination) of the nanoparticle from the environment.

In some embodiments, the polymeric matrix material is non-biodegradable. In some embodiments, the polymeric matrix material is cross-linked to slow or eliminate any degradation of the particle during use. For example, the polymeric matrix material can be cross-linked polyacrylic acid.

Suitable paramagnetic metal ions for use with the presently disclosed contrast agents include ions formed by transition elements, lanthanides, and actinides. In some embodiments, the paramagnetic metal ion comprises an elements selected from the group consisting of scandium, titanium, vanadium, chromium, manganese, iron, cobalt, nickel, copper, molybdenum, ruthenium, cerium, praseodymium, neodymium, promethium, samarium, europium, gadolinium, terbium, dysprosium, holmium, erbium, thulium, and ytterbium. In some embodiments, the paramagnetic metal ion is selected from the group consisting of gadolinium(III) (i.e. Gd³⁺) and manganese(II) (i.e., Mn²⁺).

Generally, the contrast agents of the presently disclosed subject matter will comprise a large number of paramagnetic metal ions. In some embodiments, the contrast agent can comprise a nanoparticle comprising at least one thousand paramagnetic metal ions. In some embodiments, the nanoparticle can comprise at least 25,000 paramagnetic metal ions. In some embodiments, the nanoparticle can comprise at least 60,000 paramagnetic metal ions.

As shown below in Scheme 1, the functionalized chelating groups of the presently disclosed nanoparticles comprise at least two groups: (a) a metal chelating ligand (Che) and (b) a reactive moiety (Rx). The metal chelating ligand and the reactive moiety can be linked (e.g., covalently), if necessary, by a linker group (L), which can comprise a bivalent chemical moiety such as an alkylene group or a phenylene group. In some embodiments, the functionalized chelating group comprises more than one reactive moiety.

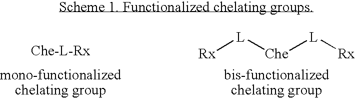

Thus, in some embodiments, the functionalized chelating ligand can bond to two or more other groups, including one or more sites on the polymeric matrix material, or to one or more other functionalized chelating ligands, optical imaging agents, targeting agents, solubility enhancing agents, circulation half-life enhancing agents, and the like. In particular, in some embodiments the functionalized chelating group can bond with multiple groups on the polymeric matrix. In some embodiments, the functionalized chelating group can bond to a site on the polymeric matrix and to the reactive moiety of another functionalized chelating group. In some embodiments, the functionalized chelating group can bond to the reactive moieties of a plurality of other functionalized chelating groups.

A number of suitable metal chelator ligands (Che) are known in the art and can be used in the nanoparticles of the presently disclosed subject matter. For example, the metal chelating ligand can comprise a polyaminocarboxylate or polyaminophosphonate group. In some embodiments, the metal chelating ligand is diethylenetriamine pentaacetate (DTPA), diethylenetriamine tetraacetate (DTTA) or 1,4,7,10-tetraazacyclododecane′-1,4,7,10-tetracetic acid (DOTA), which are examples of polyaminocarboxylate chelators. The structure of DTPA is shown in Scheme 2. Generally, the nitrogen atoms and the negatively charged carboxylate ions of these chelators can coordinate to sites on metal ions, such as Gd³⁺, therefore chelating and detoxifying them. The stability constant (K) (also referred to as the “formation constant) for Gd(DTPA)²⁻ is very high (logK=22.4) (the higher the logK, the more stable the complex). This thermodynamic parameter indicates that the fraction of Gd³⁺ ions that are in the unbound state will be quite small. For more information about the use of Gd(DTPA)²⁻ see, e.g., Caravan et al., Chemical Reviews, 99, 2293-2352 (1999); Runge et al., Magn, Reson. Imag., 3, 85 (1991); Russell et al., AJR, 152, 813 (1989); Meyer et al., Invest. Radiol., 25, S53 (1990)). For more on the use of DOTA and its derivatives as metal chelators, see U.S. Pat. Nos. 5,155,215; 5,087,440; 5,219,553; 5,188,816; 4,885,363; 5,358,704; 5,262,532; and Meyer et al., Invest. Radiol., 25, S53 (1990).

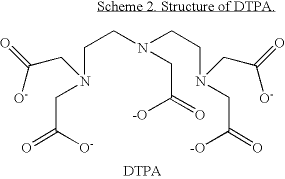

Many other metal chelators are known. See, for example, PCT

International Patent Publication No. WO96/23526, herein incorporated by reference in its entirety. Thus, in addition to DTPA, DTTA, and DOTA, any other metal chelating ligand or derivative thereof can be used in the presently disclosed nanoparticle contrast agents. These other chelators include, but are not limited to, 1,2,7,10-tetraazacyclododecane-1,4,7-triacetic acid (DO3A), trans-1,2-cyclohexanediamine tetraacetic acid (CDTA), ethylenediaminetetraacetic acid (EDTA), and tris-(2-aminoethyl)amine (TETA).

Suitable reactive moieties (Rx) for the functionalized chelating groups include any group that will react with groups on other components of the presently disclosed nanoparticles. In some embodiments, the reactive moiety will be a moiety that can react with a polymerizable monomer of the polymeric matrix material under the same or similar conditions as those used to polymerize the polymeric matrix material. Thus, in some embodiments, the reactive moiety is a vinyl group (i.e., a carbon-carbon double bond) or a siloxy group. The functionalized chelating group can include two or more different reactive moieties (i.e., moieties of two different chemical structures). For example, the functionalized chelating group can include both a vinyl group and a siloxy group, such that it can be selectively reacted with a plurality of different groups. The reactive moiety can be a group already present on the metal chelating group or can be a group attached specifically to the chelating group for use in embodiments of the presently disclosed subject matter.

The reactive moieties can be attached directly at sites on the chelating group or can be attached through a linker that is attached to a site on the chelating group. For instance, if the chelator is DTPA, the linker group can be attached at a carbon atom of one of the ethylene groups or to one of the nitrogen atoms. The reactive moiety or moieties and/or the linker or linkers can be attached to the chelator group in any suitable fashion so long as their presence does not interfere with the formation of a coordination complex between the chelator and a metal ion.

In some embodiments, the functionalized chelating group is selected from aminopropyl(trimethoxysilyl)diethylenetriamine tetraacetate (mono(APS)DTTA), bis(amino-propyltriethoxysilyl)diethylenetriamine pentaacetate (bis(APS)DTPA), bis(2-aminoethylmethacrylate)-diethylenetriamine pentaacetic acid, bis(aminopropyltrimethoxysilyl)-1,4,7,10-tetraazacyclododecane-1,4,7,10-tetraacetic acid (bis(APS)DOTA), and aminopropyl(trimethoxysilyl)-1,4,7,10-tetraazacyclododecane-1,4,7,10-tetraacetic acid (mono(APS)DOTA).

In some embodiments, the functionalized chelating groups can comprise at least one biodegradable linkage. For example, the linker group can include an amide, ester, carbamate (e.g., an aryl carbamate) or disulfide linkage. The biodegradable linkage can be a linkage that breaks (e.g. by hydrolysis, reduction, or by homolytic or heterolytic bond cleavage) in response to a change in pH or via enzyme catalysis. The pH change or enzyme can be associated with a given biological site (e.g., tissue, biological fluid, cell, or intracellular structure) or with a particular disease (e.g., cancer, inflammation). In some embodiments, the biodegradable linkage is a disulfide (R—S—S—R). The disulfide linkage is unstable in reducing environments, such as inside cells (i.e., in cytosol). Thus, in some embodiments, the biodegradable linkage of the functionalized chelating group can be degraded when the nanoparticles are taken up into cells, thereby releasing the coordination complexes from the nanoparticle.

In some embodiments, the polymeric matrix material and the coordination complexes form a copolymer. In particular, the copolymer can be formed through a reaction between reactive moieties on a functionalized chelating group and a group on the polymeric matrix material. In some embodiments, the reactive moiety on the functionalized chelating group will match the reactive functionality of the monomer used to prepare the polymeric matrix material. Thus, if the polymeric matrix material is a polyolefin, the reactive moiety of the functionalized chelating group can be a vinyl group. When the polymeric matrix material is a siloxane, the reactive moiety of the functionalized chelating group can be a siloxy group (i.e., a silyl ether). The coordination complexes can be attached to the polymeric matrix material throughout the entire volume of the matrix material. Thus, the coordination complexes can be present throughout (i.e., dispersed throughout) the core of the nanoparticle structure.

In some embodiments, the coordination complexes can be bound to the polymeric matrix material only at a terminus of the polymeric matrix material. Thus, when the polymeric matrix material comprises the core of the nanoparticle agent, the coordination complexes can be grafted onto (i.e., bound to) the outer surface of the nanoparticle. When the polymeric matrix material has been doped with a luminophore and coordination complexes are grafted to the outer surface of the nanoparticle, the resulting multimodal nanoparticle imaging agent has a luminescent core for optical imaging and a paramagnetic exterior for MR imaging.

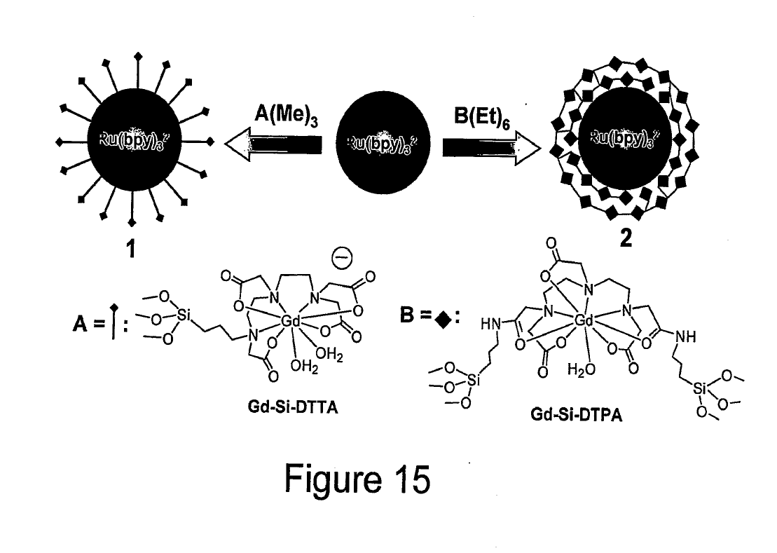

In some embodiments, only a single coordination complex is attached to a particular point on the outer surface of the nanoparticle. In some embodiments, for example, when the functionalized chelating group comprises at least two reactive moieties, the coordination complexes are not only grafted onto the outer surface of the polymeric matrix material, they further form a polymeric layer of coordination complexes that surrounds the polymeric matrix material core. Thus, the particle comprises a block co-polymer of polymeric matrix material and coordination complex. See, for example, 2, in FIG. 15.

In some embodiments, the coordination complexes are both dispersed throughout the polymeric matrix material and are bound to the surface of the particle. In some embodiments, the coordination complexes are both dispersed throughout the polymeric matrix material and form an outer polymeric layer of coordination complex.

In some embodiments, the nanoparticle can include groups, for example dispersed within or grafted to the surface of the polymeric matrix, to enhance the solubility or the ability to functionalize the polymeric matrix material. For instance, in some embodiments, the nanoparticle can comprise one or more anionic groups to enhance the aqueous solubility of the nanoparticles. Suitable anionic groups include, but are not limited to, sulfonate groups (—SO₄⁻), carboxylate groups, and phosphate groups.

In some embodiments, the nanoparticle can comprise a layer (e.g., an outer layer or an interior layer) comprising a polyanionic polymer. For instance, in some embodiments, the nanoparticle can comprise a layer comprising poly(styrene sulfonate) (PSS). In some embodiments, the PSS layer is an outer layer. In some embodiments, the polymeric matrix material can comprise a co-polymer of PSS and another polymer formed form a monomer with vinyl groups such as polypropylene, polyethylene or polyacrylic acid.

In some embodiments, the contrast agent comprises a plurality of layers including a first layer (i.e. the innermost layer of a spherical particle), which comprises the polymeric matrix material and at least some of the plurality of coordination complexes; and a second layer disposed over the first layer, the second layer comprising at least some of the plurality of coordination complexes. In some embodiments, the coordination complexes of the first layer are bound to the surface of the polymeric matrix material. In some embodiments, the second layer comprises a polymer formed from a bis-functionalized chelating group.

In some embodiments, the nanoparticle further comprises a third layer disposed over the second layer, said third layer comprising anionic groups. In some embodiments, the third layer comprises poly(styrene sulfonate) (PSS).

In some embodiments, the nanoparticle further comprises a fourth layer disposed over the third layer, wherein the fourth layer comprises at least some of the plurality of coordination complexes. In some embodiments, the fourth layer comprises a polymer formed from a bis-functionalized chelating group. In some embodiments, the fourth layer has a net positive charge or comprises positively charged groups.

The nanoparticle can comprise any number of additional layers (i.e., 1, 2, 3, 4, 5, 6, 7, 8, 9, 10, 11, 12, etc) in addition to the above-described first, second, third and fourth layers. Each of these additional layers can comprise either some of the plurality of coordination complexes, anionic groups, or a mixture thereof. The additional layers can be disposed such that each layer comprising some of the plurality of coordination complexes is the outermost layer of the nanoparticle and is disposed over a layer of anionic groups or is an inner layer of the nanoparticle and is disposed between two layers of anionic groups; and each layer comprising anionic groups is either the outermost layer of the nanoparticle and is disposed over a layer comprising some of the plurality of coordination complexes or is an inner layer of the nanoparticle and is disposed between two layers, each comprising some of the plurality of coordination complexes. Various organic or inorganic luminophores can be doped into the different layers during synthesis to aid in the use of the nanoparticles as multimodal imaging agents.

In some embodiments, the nanoparticle is approximately spherical in shape, although other shapes (i.e., disc-shaped, irregular, rod-shaped, pyramidal, cubic, etc.) are also possible. In some embodiments, the nanoparticle is approximately spherical and has a diameter of about 200 nm or less. In some embodiments, the diameter is 150 nm or less. In some embodiments, the diameter is 120 nm or less. In some embodiments, the diameter is about 100 nm or less. In some embodiments, the diameter is about 50 nm or less. In some embodiments, the diameter is between about 80 nm and about 20 nm. In some embodiments, the diameter is between about 50 nm and about 20 nm. In some embodiments, the diameter is less than 20 nm (i.e., between 19 nm and about 0.5 nm). In some embodiments, the size of the nanoparticle can be tailored based upon the desired biological target of the nanoparticle. For example, when the contrast agent is used to detect coronary artery disease, the size of the particle can be tailored to detect arterial blockages based on the size of the artery targeted or upon a pre-determined level of plaque deposits present in an artery or other blood vessel.

In some embodiments, the contrast agent can also comprise an additional moiety or moieties to further tailor their use for detecting a particular disease or for imaging a particular tissue, organ, cell, or sub-cellular structure. These additional moieties can be selected from the group consisting of a targeting agent, a solubility-enhancing agent, a circulation half-life enhancing agent, and a combination thereof.

In embodiments using a specific targeting or other additional moiety, the additional moiety can optionally be associated with the exterior (i.e., outer surface) of the particle. The targeting moiety can be conjugated (i.e., grafted or bonded) directly to the exterior via any useful reactive group on the exterior, such as, for example, an amine, an alcohol, a silyl ether, a carboxylate, an isocyanate, a phosphate, a thiol, a halide, or an epoxide. For example, a targeting moiety containing or derivatized to contain an amine that is not necessary for the recognition of the targeted cell or tissue can be coupled directly to a reactive group (e.g., a carboxylate) present on the particle exterior using carbodiimide chemistry. Synthetic linkers can be used to attach the targeting moiety to the nanoparticle surface, as well. For example, a synthetic linker containing a carboxylate or other suitable reactive group can be grafted onto the surface of the nanoparticle prior to conjugation to the additional moiety. Thus, a linker can be used to provide the nanoparticle surface with an appropriate reactive group for conjugation with a targeting or other moiety if a suitable reactive moiety is not provided by the chemical structure of the polymeric matrix material.

In some embodiments, the contrast agent can be bound to a targeting group that acts to direct the contrast agent to a specific tissue or cell type. Thus, the targeting group can cause the contrast agent, once introduced into a subject, to locate or concentrate in a specific organ or at cells expressing specific molecular signals, such as certain cancer cells. Suitable targeting groups include, but are not limited to, small molecules, polynucleotides, peptides, and proteins, including antibodies and antibody fragments, such as Fab's. In some embodiments, the targeting agent is an anti-major histocompatibility complex (MHC)-II antibody, which can target sites of inflammation.

In some embodiments, the additional moiety is a targeting agent that targets a tumor. Such tumor related targeting agents can be related to various known tumor marker or to enzymes related to a particular type of tumor. Thus, tumor targeting agents can include antibodies, antibody fragments, cell surface receptor ligands, and the like. Further targeting agents are discussed hereinbelow.

In some embodiments, the additional moiety affects the solubility or circulation half-life of the nanoparticle. For example, in some embodiments, charged groups or hydrophilic groups, including charged or hydrophilic polymers, can be grafted to the outer surface of the nanoparticle to enhance the nanoparticles solubility in aqueous environments, such as blood or plasma. In some embodiments, a more amphilphilic or hydrophobic group can be attached to the surface of the nanoparticle to enhance the lipid (or fat) solubility of the nanoparticles. In some embodiments, a group, such as a biocompatible polymer, can be attached to the outer surface of the nanoparticle to increase the size, and, therefore, the circulation half-life, of the nanoparticle. Tailoring the size of the nanoparticle can also affect the biodistribution or MRI relaxivity of the particle.

These additional groups can be biodegradable or non-biodegradable. Biodegradable polymers that could be used include poly(L-lactic acid), polycaprolactone, poly(lactide-co-glycolide), poly(hydroxybutyrate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g., PEO/PLA), polyalkylene oxalates, polyphosphazenes and biomolecules such as fibrin, fibrinogen, cellulose, starch, collagen and hyaluronic acid. Non-biodegradable polymers with a relatively low chronic tissue response such as polyurethanes, silicones, and polyesters could be used. Other non-biodegradable polymers include polyisobutylene and ethylene-alpha-olefin copolymers; acrylic polymers and copolymers, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins, polycarbonates; polyoxymethylenes; polyimides; polyethers; epoxy resins, polyurethanes; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose.

In some embodiments, the additional moiety includes a polyethylene glycol (PEG)-based polymer. PEG polymers are widely commercially available (e.g., from Aldrich Chemical Company, Milwaukee, Wis., United States of America) in a variety of sizes and with a variety of terminal functionalities to aid in their covalent attachment to the presently disclosed contrast agents. PEG is generally hydrophilic, non-biodegradable, and non-immunogenic. In some embodiments, the PEG-based polymer is polyethylene oxide (PEO)-500.

### III. Representative Uses of Hybrid Nanoparticles

III.A. Magnetic Resonance Imaging and Multimodal Imaging

In some embodiments, the presently disclosed subject matter provides a method of imaging a sample, such as but not limited to a cell, a tissue, or a subject. In some embodiments the imaging involves the use of magnetic resonance imaging (MRI). In some embodiments, the imaging involves the use of an optical imaging technique. In some embodiments, the imaging is multimodal and involves the use of both MRI and an optical imaging technique.

In some embodiments, the method of imaging comprises (a) administering to a sample, such as but not limited to a cell, a tissue, and a subject a contrast agent, said contrast agent comprising a hybrid nanoparticle, said hybrid nanoparticle comprising: a polymeric matrix material; and a plurality of coordination complexes, each coordination complex comprising a functionalized chelating group and a paramagnetic metal ion; and (b) rendering a magnetic resonance image of the one of a cell, a tissue, and a subject.

As noted hereinabove, the presently disclosed nanoparticles can comprise large numbers of paramagnetic metal ions per particle. The presently disclosed contrast agents can also exhibit very large relaxivities (r1 and/or r2) on a per mM of metal basis compared with known MRI agents comprising only a chelating agent and a paramagnetic metal ion. The presently disclosed contrast agents can also exhibit large relaxivities on a per mM of particle basis. Thus, in some embodiments, it will be possible to reduce the amount of contrast agent needed to image a given sample.

In some embodiments, the contrast agent of the presently disclosed subject matter has a longitudinal relaxivity (r1) of about 7.0 mmol⁻¹ s⁻¹ or greater, calculated based on metal ion concentration. In some embodiments, the contrast agent has an r1 of about 19.7 mmol⁻¹ s⁻¹ or greater, calculated based on metal ion concentration. In some embodiments, the r1 calculated based on nanoparticle concentration is about 2×10⁵ mmol⁻¹ s⁻¹ or greater. In some embodiments, the r1 calculated based on nanoparticle concentration is about 4.9×10⁵ mmol⁻¹ s⁻¹ or greater.

In some embodiments, the contrast agent has a transverse relaxivity (r2) of about 10 mmol⁻¹s⁻¹ or greater, calculated based on metal ion concentration. In some embodiments, the contrast agent has an r2 of about 60 mmol⁻¹ s⁻¹ or greater, calculated based on metal ion concentration. In some embodiments, the r2 calculated based on nanoparticle concentration is about 6.1×10⁵ mmol¹ s⁻¹ or greater. In some embodiments, the r2 calculated based on nanoparticle concentration is about 7.8×10⁵ mmol⁻¹ s⁻¹ or greater.

As described hereinabove, in some embodiments, the hybrid nanoparticle further comprises one or more luminophore (e.g., a fluorophore). Therefore, in some embodiments, the method of imaging a cell, tissue or subject comprises rendering an optical image of the cell, tissue or subject. In some embodiments, the method comprises both rendering an MR image and an optical image.

In some embodiments, the contrast agent is designed to be taken up into a cell or tissue, and the method of imaging the contrast agent provides a method of imaging the uptake of the contrast agent into the cell or tissue.

In some embodiments, the imaging is target-specific, wherein the contrast agent concentrates to or labels a specific sample population (e.g., a specific type of cell or tissue, such as cells of a particular organ, or cells that express markers for a particular disease). The target specificity can be based on the size of the nanoparticle or on the identity of a targeting agent associated with the contrast agent. For example, a targeting agent can be associated with the outer surface of the nanoparticle.

The MRI imaging and the optical imaging can be performed at about the same time or can be performed minutes, hours, days, or weeks apart. Several sequential images (either MRI, optical, or both) can be rendered of the same biological sample (i.e., the cell, tissue, or subject). These sequential images can be taken seconds, minutes, hours, days, weeks, or months apart. Such sequential imaging can allow for detection of the uptake and/or degradation or elimination of the contrast agent.

In some embodiments, the imaging is of a cell or tissue that is derived from, but is not present in, a living subject. In some embodiments, the imaging is of a subject, wherein the subject is a living subject. Thus, the imaging is in vivo imaging. The subject can be any animal, plant or microorganism. In some embodiments, the subject is a bird or mammal. In some embodiments, the subject is a human.

The contrast agent can be delivered as part of a formulation containing the nanomaterial and a pharmaceutically acceptable carrier (e.g., a carrier pharmaceutically acceptable in humans). Administration of the formulation can be done systemically or locally to a region of interest. The administration can comprise oral, nasal, intravenous, intramuscular, intratumoral, or intraperitoneal administration.

III.B Disease Detection

In some embodiments, the presently disclosed subject matter provides a method of detecting a disease state in one of a cell, a tissue and a subject, the method comprising: (a) administering to one of a cell, a tissue, and a subject a contrast agent, said contrast agent comprising a hybrid nanoparticle, said hybrid nanoparticle comprising: a polymeric matrix material; and a plurality of coordination complexes, each coordination complex comprising a functionalized chelating group and a paramagnetic metal ion; and (b) rendering a magnetic resonance image of the one of a cell, a tissue and a subject. In some embodiments, the nanoparticle can further comprise an optical imaging agent (e.g., a luminophore) and the method can include an optical imaging step in addition to, or as an alternative to, the MR imaging step.

In some embodiments, the subject is a living subject, such as a bird or mammal. In some embodiments, the subject is a human.

In some embodiments, the disease state can be one of cancer, cardiovascular disease (e.g., atherosclerosis, etc.), and a disease associated with inflammation (e.g. rheumatoid arthritis).

In some embodiments, the method can be used to detect the presence or absence of a disease, the location, extent, or progression of a disease, or the regression of a disease in response to a therapeutic treatment. Thus, in some embodiments, the use of the presently disclosed contrast agents can be used to help guide a health care professional in evaluating a therapeutic course of treatment (e.g., the use of one or more therapeutic agents (i.e., drugs), surgery, a diet, an exercise plan, a radiation course, etc.). In some embodiments, the contrast agents can be used to help the health care professional diagnose a disease or plan future courses of therapeutic treatment. In some embodiments, the contrast agents can be used in the course of preventative patient care, for example, to check for the occurrence of a disease in a patient at risk of developing the disease.

Diseases associated with inflammation include, but are not limited to rheumatoid arthritis, Alzheimer's disease, multiple sclerosis, chronic active hepatitis, primary biliary cirrhosis, encephalitis, meningitis, chronic viral hepatitis (i.e., as caused by Hepatitis B and Hepatitis C viruses), drug or alcohol induced hepatitis, sarcoidosis, pulmonary fibrosis, Guillaine Barre syndrome, systemic lupus erythematosus, Crohn's disease, ulcerative collitis, Reiter's syndrome, seronegative arthritis or spondylitis, vasculitis, cardiomyopathy, uveitis, nephritis, psoriasis, pneumonitis, Sjogren's syndrome, and scleroderma.

The term “cancer” as used herein refers to diseases caused by uncontrolled cell division and the ability of cells to metastasize, or to establish new growth in additional sites. The terms “malignant”, “malignancy”, “neoplasm”, “tumor” and variations thereof refer to cancerous cells or groups of cancerous cells.

Specific types of cancer include, but are not limited to, skin cancers, connective tissue cancers, adipose cancers, breast cancers, lung cancers, stomach cancers, pancreatic cancers, ovarian cancers, cervical cancers, uterine cancers, anogenital cancers, kidney cancers, bladder cancers, colon cancers, prostate cancers, central nervous system (CNS) cancers, retinal cancer, blood, and lymphoid cancers.

Thus, in some embodiments the method detects the presence of a tumor or neoplasm. Representative neoplasms that can be detected by the instant methods are selected from the group consisting of benign intracranial melanomas, arteriovenous malformation, angioma, macular degeneration, melanoma, adenocarcinoma, malignant glioma, prostatic carcinoma, kidney carcinoma, bladder carcinoma, pancreatic carcinoma, thyroid carcinoma, lung carcinoma, colon carcinoma, rectal carcinoma, brain carcinoma, liver carcinoma, breast carcinoma, ovary carcinoma, solid tumors, solid tumor metastases, angiofibromas, retrolental fibroplasia, hemangiomas, Karposi's sarcoma, and combinations thereof.

In some embodiments, the nanoparticle can comprise a targeting agent to direct the nanoparticle, once administered, to a target diseased cell. Any targeting moiety known to be located on the surface of the target diseased cells (e.g. tumor cells), or expressed by the diseased cells, finds use with the presently disclosed particles. For example, an antibody directed against a cell surface moiety can be used. Alternatively, the targeting moiety can be a ligand directed to a receptor present on the cell surface or vice versa. Thus, targeting moieties include small molecules, peptides, and proteins (including antibodies or antibody fragments (e.g., FABs)).

Targeting moieties for use in targeting cancer cells can be designed around tumor specific antigens including, but not limited to, carcinoembryonic antigen, prostate specific antigen, tyrosinase, ras, HER2, erb, MAGE-1, MAGE-3, BAGE, MN, gp100, gp75, p97, proteinase 3, a mucin, CD81, CID9, CD63; CD53, CD38, CO-029, CA125, GD2, GM2 and O-acetyl GD3, M-TAA, M-fetal or M-urinary find use with the presently disclosed subject matter. Alternatively the targeting moiety can be designed around a tumor suppressor, a cytokine, a chemokine, a tumor specific receptor ligand, a receptor, an inducer of apoptosis, or a differentiating agent. Further, given the importance of the angiogenisis process to the growth of tumors, in some embodiments, the targeting moiety can be developed to target a factor associated with angiogenisis. Thus, the targeting moiety can be designed to interact with known angiogenisis factors such as vascular endothelial growth factor (VEGF). See Brannon-Peppas, L. and Blanchette, J. O., Advanced Drug Delivery Reviews, 56, 1649-1659 (2004).

Tumor suppressor proteins provided for targeting include, but are not limited to, p16, p21, p27, p53, p73, Rb, Wilms tumor (WT-1), DCC, neurofibromatosis type 1 (NF-1), von Hippel-Lindau (VHL) disease tumor suppressor, Maspin, Brush-1, BRCA-1, BRCA-2, the multiple tumor suppressor (MTS), gp95/p97 antigen of human melanoma, renal cell carcinoma-associated G250 antigen, KS ¼ pan-carcinoma antigen, ovarian carcinoma antigen (CA125), prostate specific antigen, melanoma antigen gp75, CD9, CD63, CD53, CD37, R2, CD81, C0029, TI-1, L6 and SAS. Of course these are merely exemplary tumor suppressors and it is envisioned that the presently disclosed subject matter can be used in conjunction with any other agent that is or becomes known to those of skill in the art as a tumor suppressor.

In some embodiments, targeting is directed to factors expressed by an oncogene. These include, but are not limited to, tyrosine kinases, both membrane-associated and cytoplasmic forms, such as members of the Src family, serine/threonine kinases, such as Mos, growth factor and receptors, such as platelet derived growth factor (PDDG), SMALL GTPases (G proteins) including the ras family, cyclin-dependent protein kinases (cdk), members of the myc family members including c-myc, N-myc, and L-myc and bcl-2 and family members.

Cytokines that can be targeted by the presently disclosed particles include, but are not limited to, IL-1, IL-2, IL-3, IL-4, IL-5, IL-6, IL-7, IL-8, IL-9, IL-10, ILA 1, IL-12, IL-13, IL-14, IL-15, TNF, GM-CSF, β-interferon and γ-interferon. Chemokines that can be used include, but are not limited to, M1P1α, M1P1β, and RANTES.

Enzymes that can be targeted include, but are not limited to, cytosine deaminase, hypoxanthine-guanine phosphoribosyltransferase, galactose-1-phosphate uridyltransferase, phenylalanine hydroxylase, glucocerbrosidase, sphingomyelinase, α-L-iduronidase, glucose-6-phosphate dehydrogenase, HSV thymidine kinase, and human thymidine kinase.

Receptors and their related ligands that find use in the context of the presently disclosed subject matter include, but are not limited to, the folate receptor, adrenergic receptor, growth hormone receptor, luteinizing hormone receptor, estrogen receptor, epidermal growth factor(EGF) receptor, fibroblast growth factor receptor (FGFR), and the like. For example, EGF is overexpressed in brain tumor cells and in breast and colon cancer cells. In some embodiments, the targeting moiety is selected from the group consisting of folic acid, guanidine, transferrin, carbohydrates and sugars. In some embodiments, the targeting moiety is a peptide selected from the group consisting of the amino acid sequence RGD and TAT peptides.

Hormones and their receptors include, but are not limited to, growth hormone, prolactin, placental lactogen, luteinizing hormone, follicle-stimulating hormone, chorionic gonadotropin, thyroid-stimulating hormone, leptin, adrenocorticotropin (ACTH), angiotensin 1, angiotensin II, β-endorphin, β-melanocyte stimulating hormone (β-MSH), cholecystokinin, endothelin I, galanin, gastric inhibitory peptide (GIP), glucagon, insulin, amylin, lipotropins, GLP-1 (7-37) neurophysins, and somatostatin.

The presently disclosed subject matter provides that vitamins (both fat soluble and non-fat soluble vitamins) placed in the targeting component of the nanomaterials can be used to target cells that have receptors for, or otherwise take up these vitamins. Particularly preferred for this aspect are the fat soluble vitamins, such as vitamin D and its analogues, Vitamin E, Vitamin A, and the like or water soluble vitamins such as Vitamin C, and the like.

Antibodies can be generated to allow for the targeting of antigens or immunogens (e.g., tumor, tissue or pathogen specific antigens) on various biological targets (e.g., pathogens, tumor cells, and normal tissue). In some embodiments of the presently disclosed subject matter, the targeting moiety is an antibody or an antigen binding fragment of an antibody (e.g., Fab, F(ab′)2, or scFV units). Thus, “antibodies” include, but are not limited to polyclonal antibodies, monoclonal antibodies, chimeric antibodies, single chain antibodies, Fab fragments, and a Fab expression library.

Other characteristics of the nanoparticle also can be used for targeting. Thus, in some embodiments, the enhanced permeability and retention (EPR) effect is used in targeting. The EPR effect is the selective concentration of macromolecules and small particles in the tumor microenvironment, caused by the hyperpermeable vasculature and poor lymphatic drainage of tumors. To enhance EPR, in some embodiments, the exterior of the particle can be coated with or conjugated to a hydrophilic polymer to enhance the circulation half-life of the particle and to discourage the attachment of plasma proteins to the particle.

For additional exemplary strategies for targeted drug delivery, in particular, targeted systems for cancer therapy, see Brannon-Peppas, L. and Blanchette, J. O., Advanced Drug Delivery Reviews, 56, 1649-1659 (2004) and U.S. Pat. No. 6,471,968, each of which is incorporated herein by reference in its entirety.

### IV. Formulations

The compositions of the presently disclosed subject matter comprise in some embodiments a composition that includes a pharmaceutically acceptable carrier. Any suitable pharmaceutical formulation can be used to prepare the compositions for administration to a subject. In some embodiments, the composition and/or carriers can be pharmaceutically acceptable in humans.

For example, suitable formulations can include aqueous and non-aqueous sterile injection solutions that can contain anti-oxidants, buffers, bacteriostatics, bactericidal antibiotics, and solutes that render the formulation isotonic with the bodily fluids of the subject; and aqueous and non-aqueous sterile suspensions that can include suspending agents and thickening agents. The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Some exemplary ingredients are sodium dodecyl sulfate (SDS), in one example in the range of 0.1 to 10 mg/ml, in another example about 2.0 mg/ml; and/or mannitol or another sugar, for example in the range of 10 to 100 mg/ml, in another example about 30 mg/ml; and/or phosphate-buffered saline (PBS).

It should be understood that in addition to the ingredients particularly mentioned above, the formulations of this presently disclosed subject matter can include other agents conventional in the art having regard to the type of formulation in question. For example, sterile pyrogen-free aqueous and non-aqueous solutions can be used.

### V. Subjects

The methods and compositions disclosed herein can be used on a sample either in vitro (for example, on isolated cells or tissues) or in vivo in a subject (i.e. living organism, such as a patient). In some embodiments, the subject is a human subject, although it is to be understood that the principles of the presently disclosed subject matter indicate that the presently disclosed subject matter is effective with respect to all vertebrate species, including mammals, which are intended to be included in the terms “subject” and “patient”. Moreover, a mammal is understood to include any mammalian species for which employing the compositions and methods disclosed herein is desirable, particularly agricultural and domestic mammalian species.

As such, the methods of the presently disclosed subject matter are particularly useful in warm-blooded vertebrates. Thus, the presently disclosed subject matter concerns mammals and birds. More particularly provided is imaging methods and compositions for mammals such as humans, as well as those mammals of importance due to being endangered (such as Siberian tigers), of economic importance (animals raised on farms for consumption by humans), and/or of social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the imaging of birds, including the imaging of those kinds of birds that are endangered, kept in zoos or as pets (e.g., parrots), as well as fowl, and more particularly domesticated fowl, for example, poultry, such as turkeys, chickens, ducks, geese, guinea fowl, and the like, as they are also of economic importance to humans. Thus, also provided is the imaging of livestock including, but not limited to domesticated swine (pigs and hogs), ruminants, horses, poultry, and the like.

### VI. Administration

Suitable methods for administration of a composition of the presently disclosed subject matter include, but are not limited to intravenous and intratumoral injection. Alternatively, a composition can be deposited at a site in need of imaging in any other manner, for example by spraying a composition comprising a composition within the pulmonary pathways. The particular mode of administering a composition of the presently disclosed subject matter depends on various factors, including the distribution and abundance of cells to be imaged and/or treated and mechanisms for metabolism or removal of the composition from its site of administration. For example, relatively superficial tumors can be injected intratumorally. By contrast, internal tumors can be imaged and/or treated following intravenous injection.

In one embodiment, the method of administration encompasses features for regionalized delivery or accumulation at the site to be imaged and/or treated. In some embodiments, a composition is delivered intratumorally. In some embodiments, selective delivery of a composition to a target is accomplished by intravenous injection of the composition followed by hyperthermia treatment of the target.

For delivery of compositions to pulmonary pathways, compositions of the presently disclosed subject matter can be formulated as an aerosol or coarse spray. Methods for preparation and administration of aerosol or spray formulations can be found, for example, in U.S. Pat. Nos. 5,858,784; 6,013,638; 6,022,737; and 6,136,295.

### VII. Doses

An effective dose of a composition of the presently disclosed subject matter is administered to a subject. An “effective amount” is an amount of the composition sufficient to produce adequate imaging. Actual dosage levels of constituents of the compositions of the presently disclosed subject matter can be varied so as to administer an amount of the composition that is effective to achieve the desired effect for a particular subject and/or target. The selected dosage level can depend upon the activity (e.g., MRI relaxivity) of the composition and the route of administration.

After review of the disclosure herein of the presently disclosed subject matter, one of ordinary skill in the art can tailor the dosages to an individual subject, taking into account the particular formulation, method of administration to be used with the composition, and nature of the target to be imaged and/or treated. Such adjustments or variations, as well as evaluation of when and how to make such adjustments or variations, are well known to those of ordinary skill in the art.

### VII. Synthesis of Hybrid Nanoparticles

Microemulsions, particularly, water-in-oil, or reverse, microemulsions have been used to synthesize a variety of nanophase materials such as organic polymers, semiconductor nanoparticles (see Xu and Akins, Material. Letters, 58, 2623 (2004)), metal oxides, and nanocrystals consisting of cyanide-bridged transition metal ions. See Vaucher et al. Angew. Chem. Int Ed., 39, 1793 (2000); Vaucher et al., Nano Lett., 2, 225 (2002); Uemura and Kitagawa, J. Am. Chem. Soc., 125, 7814 (2003); Catala et al., Adv. Mater., 15, 826 (2003); and Yamada et al., J. Am. Chem. Soc., 126, 9482 (2004). Reverse microemulsions are composed of nanometer scale water droplets stabilized in an organic phase by a surfactant, which can be anionic, cationic, or neutral in charge. Numerous reports on the physical properties of microemulsion systems suggest the water to surfactant ratio, referred to as the w-value (i.e., [H₂O]/[surfactant]), largely dictates the size of the reverse micelle, which is just one of many tunable properties microemulsions offer. See Wong et al., J. Am. Chem. Soc., 98,2391 (1976); White et al., Langmuir, 21, 2721 (2005); Giustini et al., J. Phys. Chem., 100, 3190 (1996); and Kumar and Mittal, eds., Handbook of Microemulsion Science and Technology; New York: Marcel Decker, 1999. For a description of the use of microemulsions in preparing silica-coated nanoparticles, see U.S. Published Patent Application No. 20060228554, which is incorporated herein by reference in its entirety.

In some embodiments, the presently disclosed subject matter provides a method of synthesizing a hybrid nanoparticle for use as an imaging contrast agent. In particular, the presently disclosed synthesis methods involve the use of microemulsions in preparing hybrid nanoparticle contrast agents. The microemulsion can be water-in-oil (i.e., reverse micelles or water droplets dispersed in oil), oil-in-water (i.e., micelles or oil droplets dispersed in water), or a bi-continuous system containing comparable amounts of two immiscible fluids. In some cases, microemulsions can be made by mixing together two non-aqueous liquids of differing polarity with negligible mutual solubility.

The immiscible liquids that can be used to make the microemulsion typically include a relatively polar (i.e., hydrophobic) liquid and a relative non-polar (i.e., hydrophillic) liquid. While a large variety of polar/non-polar liquid mixtures can be used to form a microemulsion useful in the invention, the choice of particular liquids utilized can depend on the type of nanoparticles being made. A skilled artisan can select specific liquids for particular applications by adapting known methods of making microemulsions for use in the present invention. In many embodiments, the relatively polar liquid is water, although other polar liquids might also be useful. Water is useful because it is inexpensive, readily available, non-toxic, easy to handle and store, compatible with a large number of different precipitation reactions, and immiscible in a large number of non-polar solvents. Examples of suitable non-polar liquids include alkanes (e.g., any liquid form of hexane, heptane, octane, nonane, decane, undecane, dodecane, etc.), cycloalkanes (e.g., cyclopentane, cyclohexane, etc.), aromatic hydrocarbons (e.g., benzene, toluene, etc.), and mixtures of the foregoing (e.g., petroleum and petroleum derivatives). In general, any such non-polar liquid can be used as long as it is compatible with the other components used to form the microemulsion and does not interfere with any precipitation reaction used to isolate the particles after their preparation.

Generally, at least one surfactant is needed to form a microemulsion. Surfactants are surface active agents that thermodynamically stabilize the very small dispersed micelles or reverse micelles in microemulsions. Typically, surfactants possess an amphipathic structure that allows them to form films with very low interfacial tension between the oily and aqueous phases. Thus, any substance that reduces surface tension at the interface of the relatively polar and relatively non-polar liquids and is compatible with other aspects of the presently disclosed subject matter can be used to form the microemulsion used to make nanoparticles. The choice of a surfactant can depend on the particular liquids utilized and on the type of nanoparticles being made. Specific surfactants suitable for particular applications can be selected from known methods of making microemulsions or known characteristics of surfactants. For example, non-ionic surfactants are generally preferred when an ionic reactant is used in the microemulsion process and an ionic detergent would bind to or otherwise interfere with the ionic reactant.

Numerous suitable surfactants are known. A nonexhaustive list includes soaps such as potassium oleate, sodium oleate, etc.; anionic detergents such as sodium cholate, sodium caprylate, etc.; cationic detergents such as cetylpyridinium chloride, alkyltrimethylammonium bromides, benzalkonium chloride, cetyldimethylethylammonium bromide, etc; zwitterionic detergents such as N-alkyl-N,N-dimethylammonio-1-propanesulfonates and CHAPS; and non-ionic detergents such as polyoxyethylene esters, and various tritons (e.g., (Triton-X100, Triton-X114); etc.

The concentration of surfactant used can depend on many factors including the particular surfactant selected, liquids used, and the type of nanoparticles to be made. Suitable concentrations can be determined empirically, i.e., by trying different concentrations of surfactant until the concentration that performs best in a particular application is found. Ranges of suitable concentrations can also be determined from known critical micelle concentrations.

In some embodiments of the presently disclosed subject matter provides a method of synthesizing a hybrid nanoparticle, the hybrid nanoparticle comprising a polymeric matrix material and a plurality of coordination complexes, each of the plurality of coordination complexes comprising a functionalized chelating group and a paramagnetic metal ion, the method comprising:
(a) providing a first mixture comprising a water-in-oil microemulsion system comprising water, an organic solvent, a surfactant, and a co-surfactant;
(b) adding a polymerizable monomer and a plurality of coordination complexes, each of said plurality of coordination complexes comprising a functionalized chelating group and a paramagnetic metal ion, to the first mixture to form a second mixture;
(c) mixing said second mixture for a first period of time;
(d) adding a polymerization agent to the second mixture to form a third mixture; and
(e) mixing the third mixture for a second period of time to form a hybrid nanoparticle.

According to this method, the plurality of coordination complexes can be dispersed throughout the nanoparticle (e.g., throughout the polymeric matrix material).

The method can include the additional step of precipitating the hybrid nanoparticle. In some embodiments, the precipitation can be achieved by adding an alcohol (e.g., ethanol, methanol, etc) to the third mixture.

In some embodiments, the mixing comprises stirring (e.g., using a magnetic stirrer or a mechanical stirrer). Mixing can also refer to sonication or to manual or mechanical shaking, or to any combination thereof.

In some embodiments, the surfactant is a non-ionic surfactant. In some embodiments, the surfactant is Triton-X100. In some embodiments, the co-surfactant is 1-hexanol. In some embodiments, the molar ratio of Triton-X100 to 1-hexanol ranges between about 1 and about 5.

In some embodiments, the polymeric matrix material is an inorganic polymer. In some embodiments, the polymerizable monomer is tetraethyl orthosilicate (TEOS).

When preparing nanoparticles comprising inorganic polymers, useful water to surfactant ratios (i.e., w-, the ratio of [water]/[surfactant]) for the third mixture (i.e., after the addition of the polymerization agent, which can contribute to the water content of the mixture if dissolved in an aqueous carrier) range from about 10 to about 25 (i.e., 10, 11, 12, 13, 14, 15, 16, 17, 18, 19, 20, 21, 22, 23, 24, or 25). As described hereinbelow, in the examples, varying w- can lead to variations in the size of the resulting nanoparticles.

When TEOS is the polymerizable monomer, the polymerization agent can be aqueous ammonia. Other suitable polymerization agents include aqueous hydroxide (e.g., NaOH) or hydrazine.

In some embodiments, the polymeric matrix material is an organic polymer. For example, the polymerizable monomer can be acrylic acid or lactide.

When acrylic acid is used as the polymerizable polymer, an exemplary suitable functionalized chelating group is bis(2-aminoethylmethacrylate)diethylenetriamine pentaacetic acid.

In some embodiments, such as when acrylic acid is the polymerizable monomer, a cross-linker can be added in step (b). One suitable cross-linker is trimethylolpropane triacrylate (TMPTA). In some embodiments, a redox initiator, such as potassium persulfate, can be added to step (b), as well.

When acrylic acid (or another acrylic monomer) is the polymerizable monomer, a suitable polymerization agent is tetramethylethane diamine (TMEDA). A suitable surfactant is cetyldimethyl ammonium bromide (CTAB). The microemulsion water to surfactant ratio can range from about 5 to about 15 (i.e., 5, 6, 7, 8, 9, 10, 11, 12, 13, 14, or 15).

Regardless of whether the polymeric matrix material is organic or inorganic, in some embodiments, step (b) further comprises adding a luminophore to the first mixture as part of forming the second mixture. In some embodiments, the luminophore is ruthenium(II) tris(2,2′-bipyridine) (Ru(bpy)₃²⁺). Thus, in some embodiments, the luminophore can be embedded in the polymeric matrix material or core of the nanoparticle during synthesis of the nanoparticle.

In some embodiments, the method further comprises adding one or more surface functionalization moiety to the third mixture after the second period of time, thereby forming a fourth mixture, and mixing the fourth mixture for a third period of time to form a surface functionalized hybrid nanoparticle. In some embodiments, the one or more surface functionalization moiety comprises one of a luminophore, a hydrophilic polymer, a group that can serve as a linker between the hybrid nanoparticle and a targeting moiety, a coordination complex comprising a functionalized chelating group and a paramagnetic metal ion, and combinations thereof. In some embodiments, the one or more surface functionalization moiety is selected from the group consisting of 3-[aminopropyl(trimethoxy)silyl]fluoresceine isothiocyanate (APS-FITC), and 2-[methoxy-(polyethyleneoxy)propyl]trimethoxysilane.

In some embodiments, wherein the plurality of coordination complexes are bound to the outer surface of the nanoparticle, the method of synthesizing a hybrid nanoparticle can comprise:

(a) providing a first mixture comprising a water-in-oil microemulsion system comprising water, an organic solvent, a surfactant and a co-surfactant;

(b) adding a polymerizable monomer to the first mixture to form a second mixture;

(c) mixing said second mixture for a first period of time;

(d) adding a polymerization agent to the second mixture to form a third mixture;

(e) mixing the third mixture for a second period of time;

(f) adding to the third mixture a plurality of coordination complexes, each of the plurality of coordination complexes comprising a functionalized chelating group and a paramagnetic metal ion to form a fourth mixture; and

(g) mixing the fourth mixture for a third period of time to form a hybrid nanoparticle having one or more of the plurality of coordination complexes bound to a surface of the hybrid nanoparticle.

In some embodiments of this method, step (b) further comprises adding a luminophore, such as ruthenium(II) tris(2,2′-bipyridine), to the first mixture as part of forming the second mixture. Thus, the core of the nanoparticle can comprise a luminophore. When the luminophore is Ru(bpy)₃²⁺, it can be embedded in pores in the polymeric matrix material.

In some embodiments, the method further comprises adding an alcohol (e.g., methanol, ethanol, etc.) to the fourth mixture after the third period of time, thereby precipitating the hybrid nanoparticle.

In some embodiments, the presently disclosed subject matter provides a method of synthesizing a layered hybrid nanoparticle, the method comprising:
(a) preparing a hybrid nanoparticle in a water-in-oil microemulsion, said hybrid nanoparticle comprising a polymeric matrix material and a plurality of coordination complexes, each of the plurality of coordination complexes comprising a functionalized chelating group and a paramagnetic metal ion; and
(b) adsorbing onto the hybrid nanoparticle prepared in step (a) a polymer comprising additional coordination complexes, said additional coordination complexes each comprising a functionalized chelating group and a paramagnetic metal ion to form a layer of polymerized coordination complexes over the surface of the hybrid nanoparticle.

In some embodiments, the adsorbing of step (b) comprises providing ultrasonication to a mixture of the hybrid nanoparticle and the polymer comprising additional coordination complexes.

In some embodiments, one or more of the plurality of coordination complexes is bound to a surface of the hybrid nanoparticle prepared in step (a).

In some embodiments, the method of synthesizing a layered nanoparticle further comprises contacting the layered hybrid nanoparticle with a mixture comprising an anionic polymeric material, said anionic polymeric material forming a layer over the layer of polymerized coordination complexes. In some embodiments, the anionic polymeric material is poly(styrene sulfonate) (PSS). In some embodiments, the method further comprises adding one or more additional layers to the layered hybrid nanoparticle such that the one or more additional layers are alternately a layer comprising polymeric coordination complex and a layer comprising anionic polymeric material.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Materials and Methods

Triton X-100, GdCl₃.6H₂O, 1-hexanol, hexanes, cyclohexane, tetraethyl orthosilicate (TEOS), pyridine, diethylenetriamine pentaacetic acid dianhydride, acrylic acid, trimethylolpropane triacrylate, 2-aminoethyl methacrylate, potassium persulfate, methanol, and aqueous NH₄⁺H⁻ were purchased from Aldrich (Aldrich Chemical Company, Milwaukee, Wis., United States of America) and used without further purification. 3-Aminopropyl triethoxysilane (APS), 3-(trimethoxysilylpropyl)diethylene triamine, and 2-[methoxy(polyethyleneoxy)propyl]trimethoxysilane were purchased from Gelest (Gelest, Inc., Morrisville, Pa., United States of America). Poly(sodium 4-styrene-sulfonate) (PSS, M_{w} 70,000) was purchased from Aldrich (Aldrich Chemical Company, Milwaukee, Wis., United States of America). Modified Gd-DOTA polymer was synthesized by oxidative coupling of bis(alkyne) monomers followed by hydrogenation and Gd loading. The cationic final polymer was dialyzed in dialysis tubing with MWCO 3500.

Thermogravimetric analysis (TGA) was performed using a Shimadzu TGA-50 (Shimadzu Corp., Kyoto, Japan) equipped with a platinum pan and heated at a rate of 3° C./min under air. A Hitachi 4700 field emission scanning electron microscope (SEM; Hitachi Ltd., Tokyo, Japan) and a JEM 100CX-II transmission electron microscope (JEOL Ltd., Tokyo, Japan) were used to determine particle size and morphology. Scanning electron microscope (SEM) images of the nanoparticles were taken on glass substrate. A Cressington 108 Auto Sputter Coater (Cressington Scientific Instruments, Ltd., Watford, United Kingdom) equipped with an Au/Pd (80/20) target and M™-10 thickness monitor was used to coat the sample with approximately 5 nm of conductive layer before taking SEM images. Gd³⁺ ion concentration was measured on a SpectraSpan7 Direct Current Plasma (DCP) Spectrometer (Applied Research Laboratories, La Brea, Calif., United States of America). Emission and excitation data were collected on a Shimadzu RF-5301 PC Spectrofluorophotometer. T1 and T2 values were determined on a Bruker 3.0 Tesla full body Magnetic Resonance Imaging (MRI) scanner (Bruker BioSpin MRI GmbH, Ettlingen, Germany). Confocal laser scanning microscope images were taken with a Zeiss LSM5 Pascal Confocal Laser Scanning Microscope (Carl Zeiss, Inc., Thornwood, N.Y., United States of America) or a Leica SP2 Laser Scanning Confocal Microscope (Leica Microsystems, Inc., Exton, Pa., United States of America) with 488 nm excitation and a 530 LP emission filter. Fluorescence microscope images were taken with a Zeiss Axiovert 100 TV Fluorescence Microscope (Carl Zeiss, Inc., Thornwood, N.Y., United States of America) using a FITC filter.

T1 values were obtained using the standard inversion-recovery method, whereas T2 values were determined using spin-echo pulse sequences. A series of dilutions of the nanomaterials were prepared for each system in 2 mL pure water or 0.1% Xanthan gum for which T1 and T2 data was collected. Plots of 1/T1 vs [Gd³⁺ ] were constructed from the data to determine accurate longitudinal relaxivity (r1) and transverse relaxivity (r2) values.

### Example 1

### 3-Aminopropyl(trimethoxysilyl)diethylenetriamine Tetraacetic Acid (Si-DTTA)

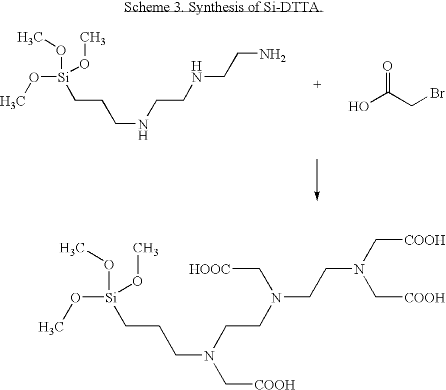

Bromoacetic acid (0.5558 g, 4.00 mmol) and 3-(trimethoxysilylpropyl)diethylene triamine (0.2654 g, 1.00 mmol) were dissolved in 1.0 mL of distilled H₂O and 2.0 mL 2M NaOH (4.00 mmol) with magnetic stirring. The reaction solution was subsequently heated to 50° C., and an additional 3.0 mL of 2M NaOH were added dropwise over approximately 30 minutes. After stirring for an additional 2 h at 50° C., the solvent was removed under reduced pressure to yield a viscous yellow oil. An off-white hygroscopic powder was isolated from the oil in high yield (>90%) by precipitation with EtOH, and subsequent drying in vacuo. MS (ESI negative ion): m/z 542.2 [M-H]⁻ for the silanetriol from a basic solution. NMR: ¹H (D₂O, 300 MHz, ppm): 0.47 (2H), 1.55 (2H), 2.62-2.78 (10H), 3.14-3.21 (8H).

### Example 2

### Synthesis of Gd—Si-DTTA Complex

The gadolinium complex was prepared by dissolving the isolated Si-DTTA product (108.6 mg, 0.2 mmol) in 4 mL H₂O with magnetic stirring at room temperature. GdCl₃ (380 μL of a 0.50 M solution, 0.19 mmol) was slowly titrated into the solution until the formed precipitate would no longer dissolve back into solution, while maintaining a pH of ˜9 with the dropwise addition of 2M NaOH. After stirring the above reaction for 2 h, Chelex 100 (Na⁺ form) was added to remove excess Gd³⁺, which was removed via filtration after 30 min. The resultant solution was then concentrated to 1 mL to yield a ˜0.20 M solution of the mono-silyl derivatized Gd complex (Gd—Si-DTTA).

### Example 3

### Bis(3-aminopropyltriethoxysilyl)diethylenetriamine pentaacetic acid (Si-DTPA)

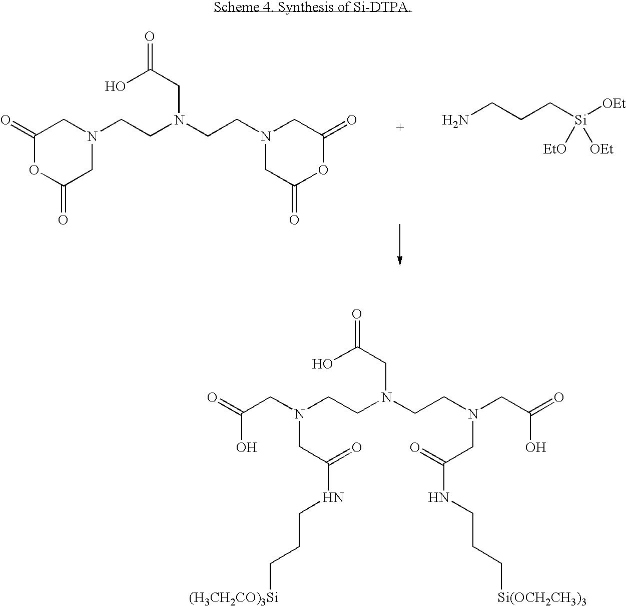

Diethylenetriamine pentaacetic acid dianhydride (5.000 g, 13.995 mmol) was dissolved in 110 mL of anhydrous pyridine under a steady flow of nitrogen. Using standard Schlenk line techniques 3-aminopropyl triethoxysilane (6.85 g, 31.00 mmol) was added and the resultant reaction mixture was magnetically stirred under nitrogen for 24 hours. The product was then precipitated with copious amounts of hexane, isolated via centrifuge, washed with additional aliquots of hexanes, and dried to yield 10.436 g (93.2%) of the desired compound (Si-DTPA). MS (ESI negative ion): m/z 631.3 [M-H]⁻ for the silanetriol from a basic solution. NMR: ¹H (DMSO, ppm): 0.52 (t, 4H), 1.14 (t, 18H), 1.44 (p, 4H), 2.81 (t, 4H), 2.92 (t, 4H), 3.04 (q, 4H), 3.22 (s, 6H), 3.34 (s, 4H), 3.73 (q, 12H), 8.06 (t, 2H). ¹³C{1H} (DMSO, ppm): 8.0 (2C), 18.8 (18C), 23.4 (2C), 41.8 (2C), 51.2 (2C), 52.8 (2C), 55.9 (2C), 56.7 (2C), 58.3 (1C), 58.4 (6C), 170.7 (2C), 173.4 (3C).

### Example 4

### Synthesis of Gd-Di-DTPA Complex

To prepare the gadolinium complex, Si-DTPA (1.77 g, 2.22 mmol) was dissolved in ˜3 equivalents of NaOH (6.0 mL of a 1.0 M solution) with magnetic stirring for 30 minutes. To this solution was added 0.90 equivalent of GdCl₃ (4.0 mL of a 0.5 M solution, 0.002 mol) and the mixture was magnetically stirred at room temperature for several hours, the volume of the solution was adjusted to 10 mL to yield a visibly clear yellow 0.20 M solution of the modified gadodiamide complex.

### Example 5

### General Synthesis and Characterization of Silica Nanoparticles

Silica nanoparticles (SNPs) were synthesized via the neutral Triton X-100/1-hexanol/cyclohexane microemulsion system. Initially, Triton X-100 (15.625 g, 0.075 mol) and 1-hexanol (38.318 g, 0.375 mol) were dissolved in cyclohexane and diluted to 250 mL to make a 0.3 M Triton X-100 stock microemulsion solution with 5 molar equivalents of the co-surfactant 1-hexanol. A typical synthesis using a w-value of ˜15 (w-=[H₂O]/[surfactant]) microemulsion system comprised adding 3.05 mL distilled H₂O and 500 μL TEOS to 50 mL of a 0.3 M Triton X-100/1.5 M1-hexanol/cyclohexane stock solution while vigorously stirring at room temperature. After 10 min of vigorous stirring, or until the microemulsion mixture became optically transparent, 1 mL of aqueous NH₄⁺H⁻ was added to initiate hydrolysis, and the resultant visibly clear microemulsion mixture was stirred for another 24 hrs before workup. During the workup, the nanoparticles were precipitated with an equivalent volume (with respect to the total microemulsion volume) of methanol, isolating the nanoparticles via centrifuge at 12500 rpm, and subsequently washing them with methanol and H₂O before redispersing them in H₂O.

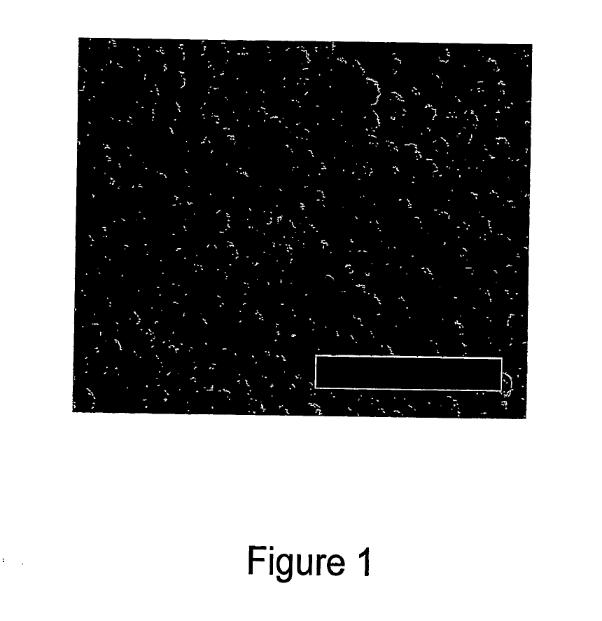

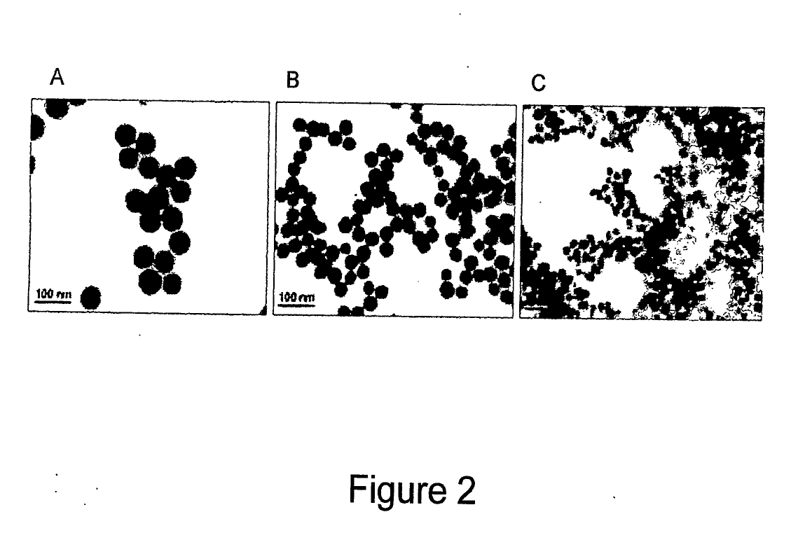

SEM images of the silica based nanoparticles formed according to this method showed that, in almost all cases, monodisperse spheres with a tunable size in the range of 20-100 nm in diameter were obtained (see FIG. 1). FIGS. 2A, 2B and 2C show TEM images of silica nanoparticles synthesized using microemulsions with different w-values.

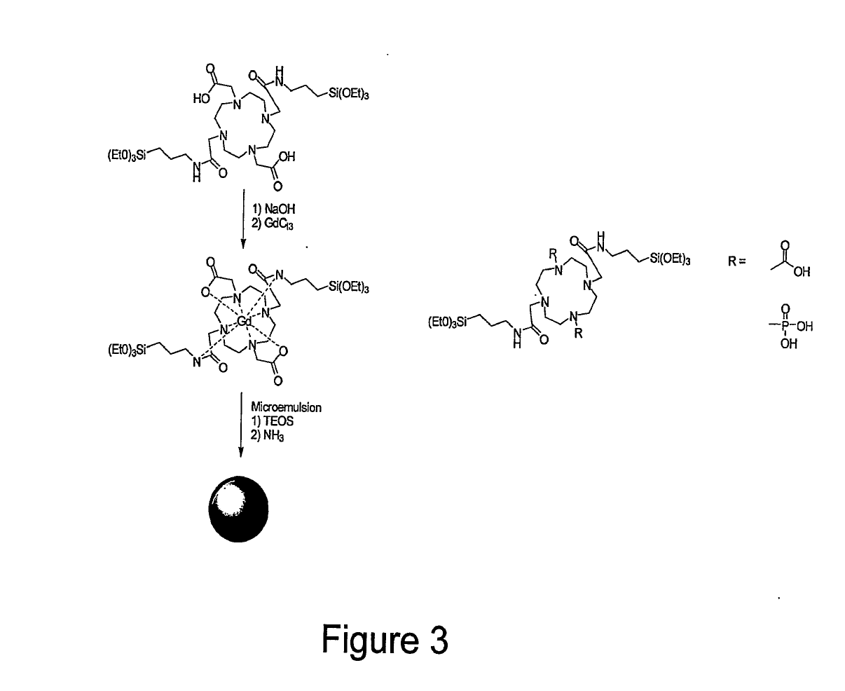

As described further, hereinbelow, similar techniques can be employed to incorporate other molecules, such as [Ru(bpy)₃]Cl₂, into the nanoparticles, such that they can be tracked with fluorescence microscopy. Additionally, this method can be adapted such that stable metal chelate complexes can be incorporated into the particles or grafted onto their surface. FIG. 3 shows a scheme illustrating the synthesis of nanoparticles comprising Gd-DOTA groups.

### Example 6

### Bis(APS)DTPA-Gd-Incorporated SNPs

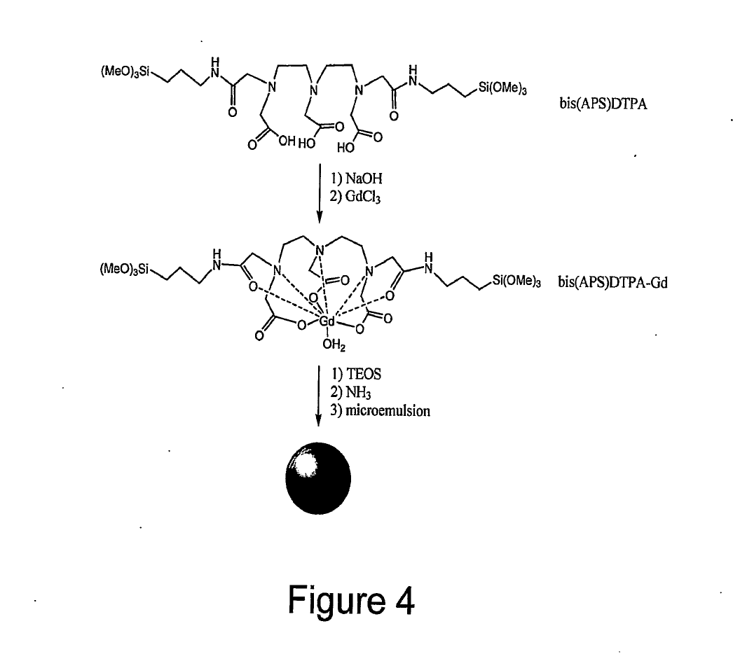

A bis-(aminopropyltriethoxy)silane (APS) derivative of the DTPA-Gd complex can be incorporated into the silica matrix during nanoparticle formation as shown in FIG. 4.

For the preparation of bis(APS)DTPA-Gd-incorporated silica nanoparticles, a w-=10 microemulsion was prepared by adding 1.75 mL distilled H₂O, 450 μL of a 0.2 M bis(aminopropyltriethoxysilyl)diethylenetriamine pentaacetate gadodiamide solution, and 500 μL TEOS to 50 mL of a 0.3 M Triton-X100/1.5 M1-hexanol/cyclohexane stock solution while vigorously stirring at room temperature. The SNPs were then precipitated with an equivalent volume of methanol and isolated via centrifuge at 10000 rpm for 20 min. The SNPs were subsequently washed twice with MeOH by redispursement via sonication and twice with H₂O before redispersing them in 5 mL of water. Approximately 65 mg of functionalized SNPs were isolated from this procedure.

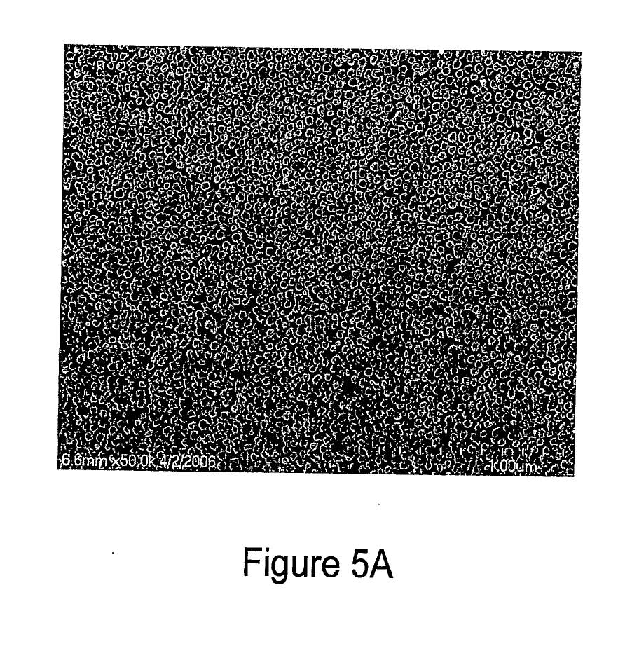

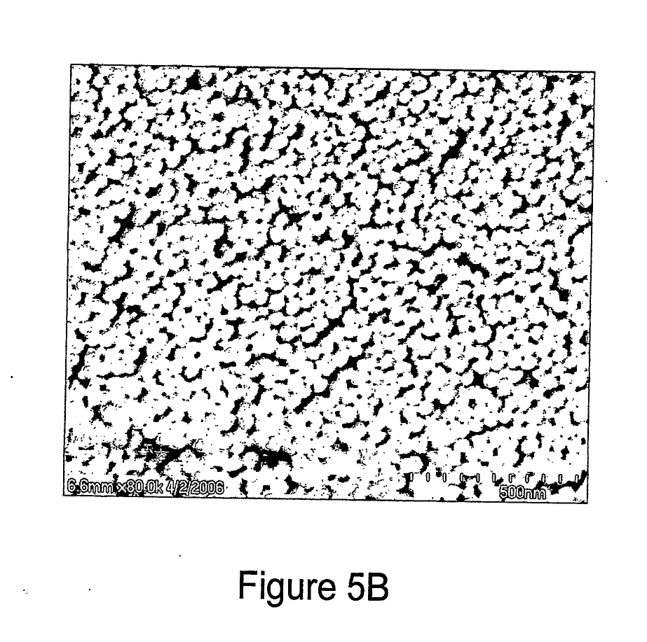

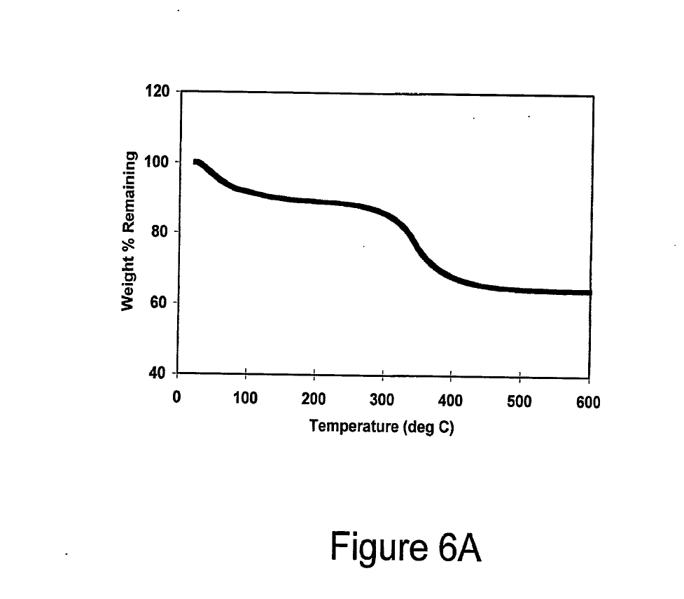

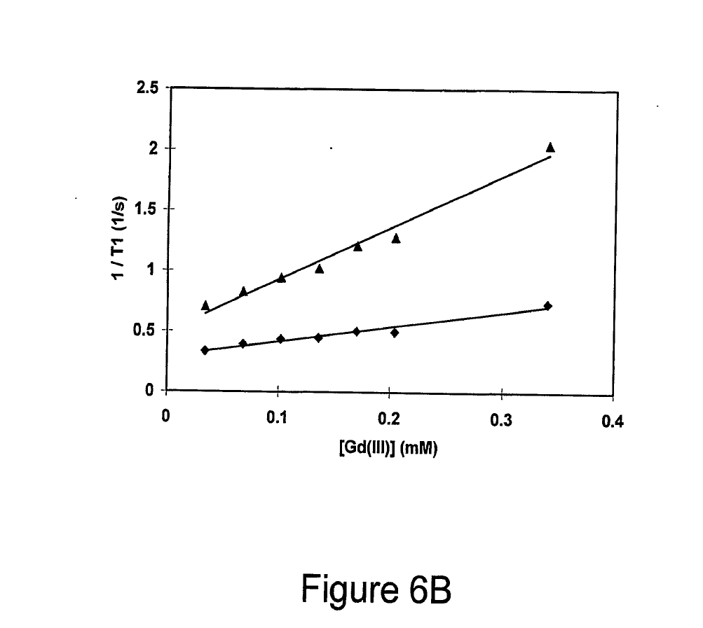

The functionalized SNPs were generally spherical with an outer diameter of approximately 40 nm as determined from SEM. See FIGS. 5A and 5B. Thermogravimetric analyses showed an initial weight loss of 11% corresponding to the loss of adsorbed solvent species and a final weight loss of 25% at approximately 300° C. corresponding to the loss of coordinating ligands. See FIG. 6A. Direct current plasma spectroscopic measurements suggested the nanoparticles were about 7.2% Gd³⁺ by mass, which corresponds to 9,000 to 18,000 Gd³⁺ per nanoparticle. The longitudinal (r1) and transverse relaxivities (r2) for the nanoparticles were determined to be 1.2 s⁻¹ and 3.4 s⁻¹ per mM of metal ion, respectively. See FIG. 6B.

### Example 7

### Ru(bpy)₃²⁺-Doped Gd—Si-DTTA-Functionalized SNPs (1)

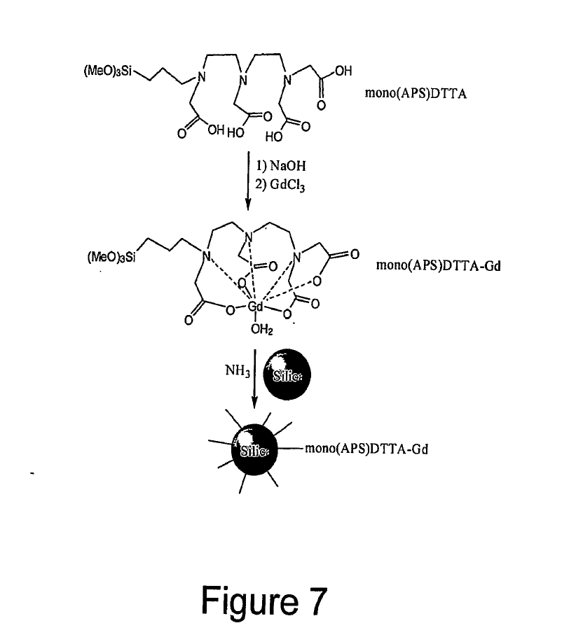

A mono(APS)DTTA-Gd derivative can be grafted onto (i.e., bound to) the surface of nanoparticles (including those with imbedded [Ru(bpy)₃]Cl₂) as shown in FIG. 7. More particularly, Ru(bpy)₃²⁺-doped SNPs were prepared by adding 2.28 mL distilled H₂O, 160 μL of a 0.1 M Ru(bpy)₃²⁺ aqueous solution, and 400 μL TEOS to 40 mL of a 0.3 M Triton X-100/1.5 M1-hexanol/cyclohexane stock solution while vigorously stirring at room temperature. After 10 min of vigorous stirring at room temperature, 0.8 mL of aqueous NH₄⁺H⁻ was added to initiate hydrolysis, and the resultant optically transparent red microemulsion mixture was stirred for another 20 hrs before adding 1.0 mL of a 0.12 M Gd—Si-DTTA aqueous solution to the reaction mixture and stirring for an additional 24 hrs. The functionalized SNPs were then precipitated with an equivalent volume of methanol and isolated via centrifuge at 12500 rpm for 30 min. The SNPs were subsequently washed twice with MeOH and twice with H₂O by re-dispersing via sonication and isolation via centrifugation. The SNPs were then re-dispersed in water. Approximately 150 mg of functionalized SNPs were isolated from this procedure.

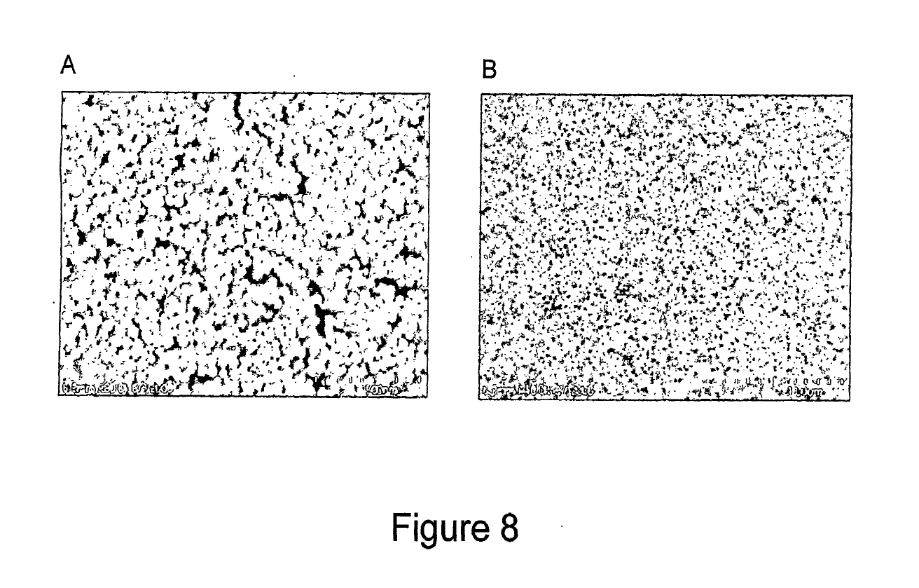

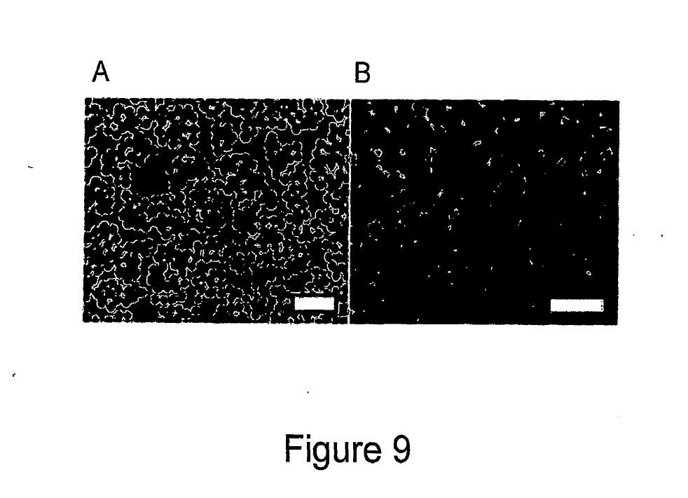

Scanning electron microscopy (SEM) and transmission electron microscopy (TEM) images indicated that nanoparticles prepared as above when w-=15 exhibit a monodisperse spherical morphology with a diameter of approximately 37 nm. See FIG. 8A, FIG. 8B, and FIG. 9A. Unless specifically stated otherwise, 1, refers to Ru(bpy)₃²⁺-doped Gd-DTTA functionalized SNPs prepared using a w-=15 microemulsion, having an average diameter of approximately 37 nm.

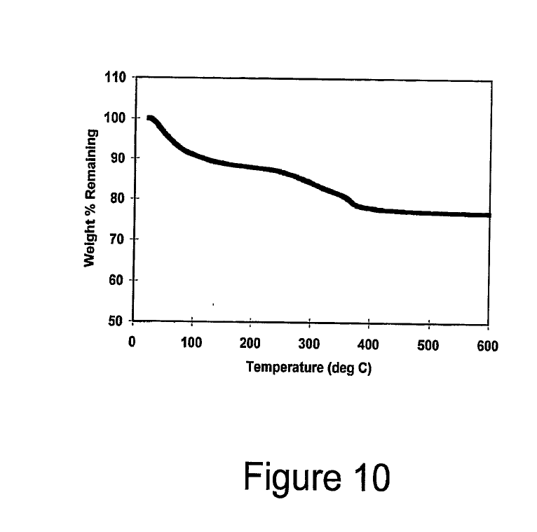

Thermogravimetric analysis of 1 showed an initial weight loss of 12% from room temperature to 180° C. and a further weight loss of 11% from 180° C. to 450° C., which corresponds to the loss of adsorbed solvent species and the loss of organic components upon the full covalent linkage of Gd-DTTA, respectively. See FIG. 10.

The TGA and DCP results correspond to a loading of about 10,200 Gd-DTTA/particle (NP), which was calculated as follows:
Concentration: 7.6 mg/mL, 2.086 mM
Mass % Gd: 4.3%
Diameter: 37 nm
Mass silica NP: (4/3)×π×(37/2)³=26,500 nm³×1×10⁻²¹ cm³=2.65×10⁻¹⁷ nm cm³; 2.65×10⁻¹⁷ nm cm³×2.0 g cm⁻³=5.30×10⁻¹⁷ g SiNP⁻¹
   # Gd per NP: (Y×m_{Gd})/m_{SiNP}+Y×m_{Gd-DTTA})=mass % Gd

(Y×157.25 g mol⁻¹)/[5.30×10⁻¹⁷×6.022×10²³ SiNP mol⁻¹)+(Y×530 g mol⁻¹)]=4.3%

Y×157.25=137×10⁶+Y×22.8

Y×134.5=137×10⁶

Y=10,200 Gd SiNP⁻¹

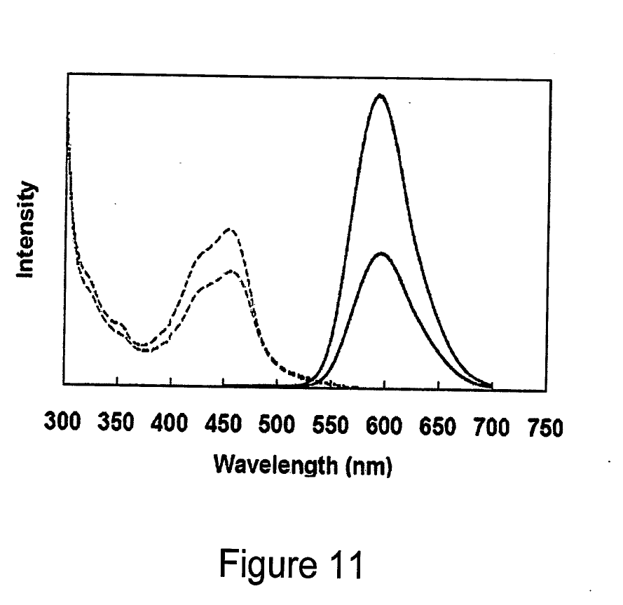

A dispersion of 1 in water showed a ligand to metal charge transfer (LMCT) absorption peak around 450 nm and an emission peak at 595 nm (λₑₓ=488 nm) for the incorporated [Ru(bpy)₃]Cl₂. See FIG. 11.

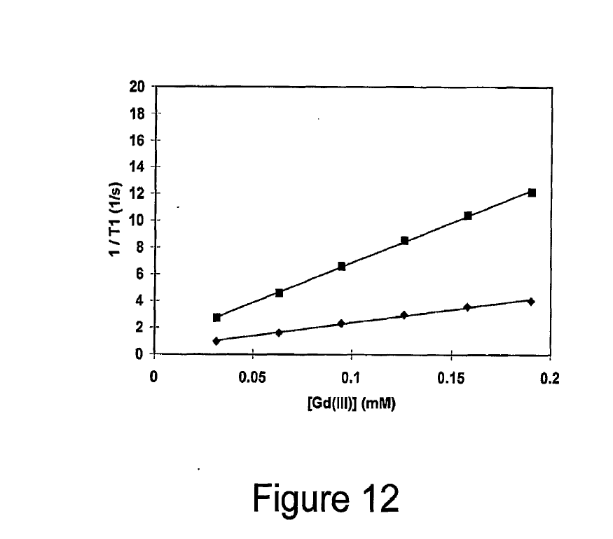

Nanoparticles of 1 were determined to have a longitudinal relaxivity (r1) of 19.7 s⁻¹ and a transverse relaxivity (r2) of 60.0 s⁻¹ on a per millimolar Gd³⁺ basis. The relaxivity curves for 1 are shown in FIG. 12. The relaxivity values of 1 are much higher than those of the Gd—Si-DTTA complex (r1=6.8 and r2=7.0 s⁻¹ mM⁻¹ Gd³⁺). Without being bound to any one particular theory, this is presumably as a result of the reduced tumbling rates of 1 in aqueous solution as compared to the smaller complex. Because of the high payload of Gd-DTTA chelates, nanoparticles of 1 display r1 and r2 values of 2.0×10⁵ s⁻¹ and 6.1×10⁵ s⁻¹, respectively, on a per millimolar particle basis.

Ru(bpy)₃²⁺-doped Gd—Si-DTTA functionalized SNPs were also made using a variation on the above-described method. In one variation, Ru(bpy)₃ doped SNPs were prepared by adding 2.85 mL distilled H₂O, 200 μL of a 0.1 M Ru(bpy)₃²⁺ aqueous solution, and 500 μL TEOS to 50 mL of a 0.3 M Triton-X100/1.5 M1-hexanol/cyclohexane stock solution while vigorously stirring at room temperature. After 10 min of vigorous stirring at room temperature, 1 mL of aqueous NH₄⁺H⁻ was added to initiate hydrolysis, and the resultant optically transparent reddish microemulsion mixture was stirred for another 12 hrs before adding 1.5 mL of a 0.1 M gadolinium (aminopropyltrimethoxysilyl) diethylenetriamine tetraacetate aqueous solution to the reaction mixture and stirring for an additional 24 hrs. The functionalized SNPs were then precipitated with an equivalent volume of methanol and isolated via centrifuge at 12,500 rpm for 20 min. The SNPs were subsequently washed twice with MeOH by redispursement via sonication and twice with H₂O before redispersing them in 10 mL of water. Approximately 86 mg of functionalized SNPs were isolated from this procedure.

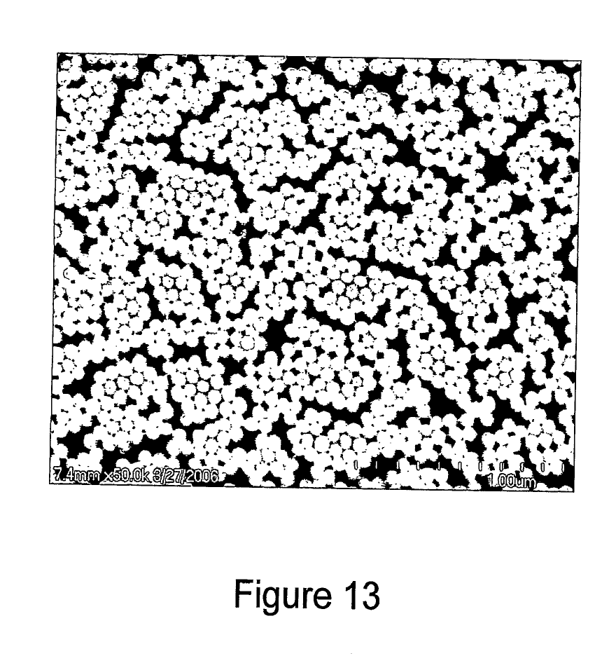

This variation of the preparation method produced slightly larger functionalized SNPs, which were spherical with an outer diameter of approximately 45 nm as determined from SEM. See FIG. 13. The bulk material was highly dispersible in aqueous solvent due, presumably, to the anionic charge on the surface of the nanoparticles. Direct current plasma spectroscopic measurements suggested the nanoparticles were approximately 3.8% Gd³⁺ by mass, corresponding to about 14,200 Gd³⁺ per particle. The TGA analysis and the r1 and r2 values for these slightly larger SNPs were the same as for 1.

### Example 8

### Ru(bpy)₃²⁺ Doped Gd—Si-DTPA-Functionalized SNPs (2)

Ru(bpy)₃²⁺-doped SNPs were prepared by adding 2.85 mL distilled H₂O, 200 μL of a 0.1 M Ru(bpy)₃²⁺ aqueous solution, and 500 μL TEOS to 50 mL of a 0.3 M Triton X-100/1.5 M 1-hexanol/cyclohexane stock solution while vigorously stirring at room temperature. After 10 min of vigorous stirring at room temperature, 1 mL of aqueous NH₄⁺H⁻ was added to initiate hydrolysis, and the resultant optically transparent red microemulsion mixture was stirred for another 24 hrs at room temperature. To a 10 mL aliquot of the above reaction mixture was added 385 μL of a 0.2 M bis(aminopropyl-triethoxysilyl)diethylenetriamine pentaacetate gadodiamide (Gd—Si-DTPA) solution and the reaction mixture was stirred for an additional 12 hrs. The Gd—Si-DTPA functionalized SNPs were then precipitated with an equivalent volume of methanol and isolated via centrifuge at 12500 rpm for 30 min. The SNPs were subsequently washed twice with MeOH by re-dispersing via sonication and twice with H₂O before re-dispersing them in 5 mL of water. Approximately 55 mg of functionalized SNPs were isolated from this procedure (using 10 mL of the above microemulsion reaction). Results suggest that when care is taken in isolating and washing the SNPs, >300 mg of nanomaterial can be isolated from a ˜54 mL microemulsion reaction.

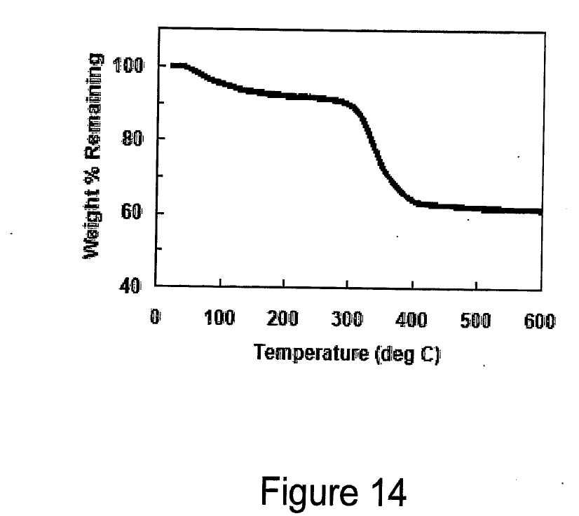

The nanoparticles, 2, formed according to the synthesis described directly above were characterized using SEM, TEM, TGA, DCP and relaxivity measurements. The nanoparticles had an average diameter of 40 nm. See FIG. 9B. TGA analysis of the particles showed an initial weight loss of 13.5% from r.t. to 180° C. for the adsorbed solvent species and a further weight loss of 33.2% from 280-450° C. for the organic components of Gd—Si-DTPA. See FIG. 14.

TGA and DCP results indicated that 2 has a loading of approximately 63,200 Gd³⁺ ions/particle, calculated as follows:
Concentration: 9.5 mg/mL, 8.37 mM
Mass % Gd: 13.9%
Diameter: 37 nm (conservative estimate)
Mass silica NP: (4/3)×π×(37/2)³=26,500 nm³×1×10⁻²¹ cm³=2.65×10⁻¹⁷ nm cm³; 2.65×10⁻¹⁷ nm cm³×2.0 g cm⁻³=5.30×10⁻¹⁷ g SiNP⁻¹
   # Gd per NP: (Y×m_{Gd})/m_{SiNP}+Y×m_{Gd-DTTA})=mass % Gd

(Y×157.25 g mol⁻¹)/[5.30×10⁻¹⁷×6.022×10²³ SiNP mol⁻¹)+(Y×628 g mol⁻¹)]=0.139

Y×157.25=4.43×10⁶+Y×87.3

Y×70.0=4.43×10⁶

Y=63,200 Gd SiNP⁻¹

This large number of metal ions suggests that, unlike Gd—Si-DTTA, Gd—Si-DTPA can form multi-layers over the core silica nanoparticle leading to a thick coating of siloxane polymer that comprises the metal chelating complex. See FIG. 15. Relaxivity measurements, however indicated that for 2, r1=7.8 s⁻¹ and r2=12.3 s⁻¹ per millimolar Gd³⁺. For comparison, the Gd—Si-DTPA complex has an r1 and r2 of 6.2 s⁻¹ and 8.0 s⁻¹ per millimolar Gd³⁺, respectively. Thus, the relaxivity values exhibited by 2 are lower than those of 1 when calculated based on metal ion concentration. Without being bound to any one particular theory, this lower relaxivity is presumably because the Gd³⁺ centers in the inner layers are not readily accessible to water molecules. Nonetheless, 2 has an impressive r1=4.9×10⁵ s⁻¹ and r2=7.8×10⁵ s⁻¹ calculated per millimole of particles, respectively.

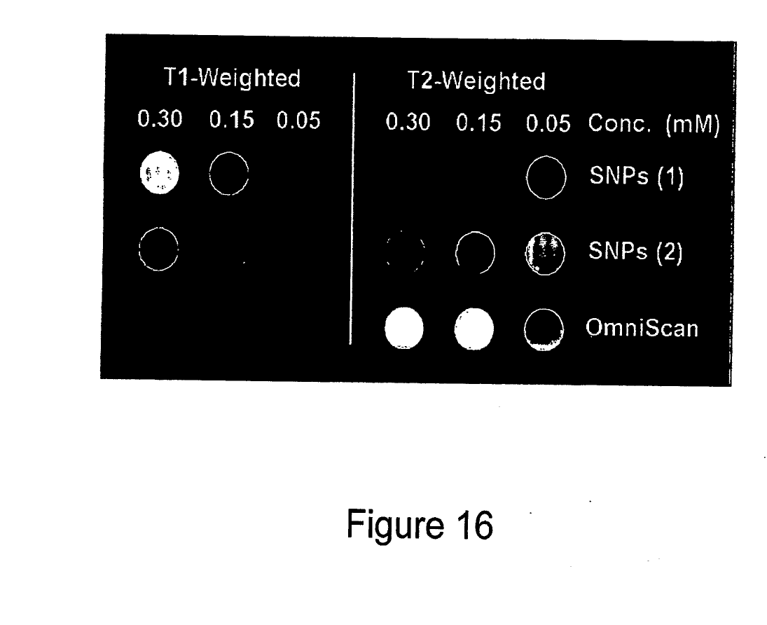

To further compare the MR imaging ability of 1 and 2, both to one another and to MRI contrast agents presently in general use, FIG. 16 shows the T1-weighted and T2-weighted phantom MR images of SNPs of 1 and 2 dispersed in water at various concentrations (0.30, 0.15, and 0.05 mM). FIG. 16 also shows the phantom MR images of the same concentrations of OMNISCAN™ (gadodiamide, the gadolinium complex of diethylenetriamine pentaacetic acid bismethylamine; available from GE Healthcare, Princeton, N.J., United States of America).

Varying the synthesis of the particles slightly, it was found that the average diameter of particles prepared with Gd—Si-DTPA was 63 nm and 22 nm using a microemulsion with a w-value of 10 and 20, respectively. The inverse dependence of particle size on the w value is likely a result of enhanced nucleation of silica particles at the reverse micelle oil-water interface since the number of reverse micelles typically increases as the w-value increases.

In one variation on the above-described synthesis, particles were formed that appeared to have fewer metal ions. For example, particles could also be formed by adding 2.85 mL distilled H₂O, 200 μL of a 0.1 M Ru(bpy)₃²⁺ aqueous solution, and 500 μL TEOS to 50 mL of a 0.3 M Triton-X100/1.5 M1-hexanol/cyclohexane stock solution while vigorously stirring at room temperature. After 10 min of vigorous stirring at room temperature, 1 mL of aqueous NH₄⁺H⁻ was added to initiate hydrolysis, and the resultant optically transparent reddish microemulsion mixture was stirred for another 12 hrs at room temperature. To a 15 mL aliquot of the above reaction mixture was added 450 μL of a 0.2 M bis(aminopropyltriethoxysilyl)diethylenetriamine pentaacetate gadodiamide solution and the reaction mixture was stirred for an additional 24 hrs. The bis(APS)DTPA-Gd-functionalized SNPs were then precipitated with an equivalent volume of methanol and isolated via centrifuge at 12500 rpm for 20 min. The SNPs were subsequently washed twice with MeOH, redispersed via sonication, and twice washed with H₂O, before being redispersed in 10 mL of water. Approximately 74.5 mg of functionalized SNPs were isolated from this procedure using 15 mL of a microemulsion reaction. Results also suggest that when care is taken in isolating and washing the SNPs up to 250 mg of nanomaterial can be isolated from a 50 mL microemulsion reaction.

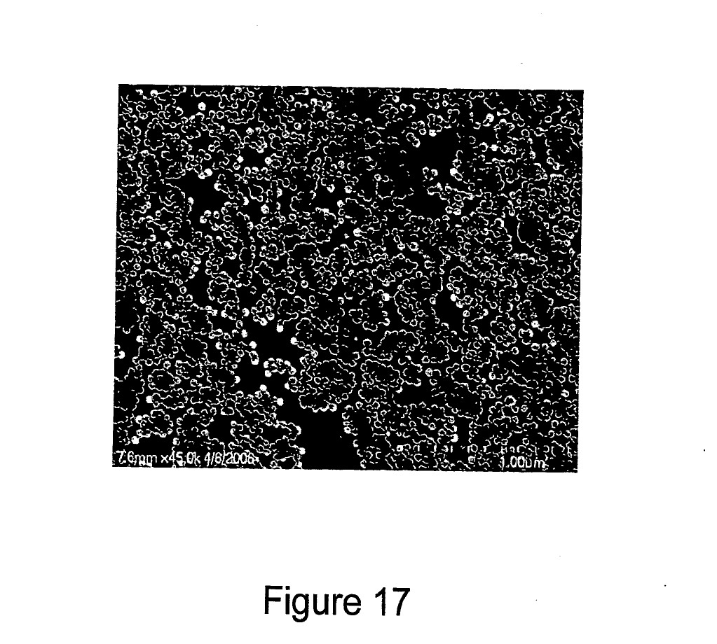

The functionalized SNPs were spherical with an outer diameter of less than 50 nm as determined from SEM (see FIG. 17), and the bulk material was highly dispersable in aqueous solvent. Without being bound to any one theory, this could be due to the porous structure generated on the surface of the nanoparticles. TGA analyses showed an initial weight loss of 8% corresponding to the loss of adsorbed solvent species and a weight loss of 31% corresponding to the loss of coordinating ligands. Calculations suggest that there are ˜25,000 Gd³⁺ per nanoparticle. The r1 these SNPs measured in aqueous solution on a Bruker 300 MHz NMR using the standard inversion recovery method was determined to be ˜10 s⁻¹ per mM of Gd³⁺.

### Example 9

### PEO-500 and APS-FITC Functionalized SNPs

PEO-500 and APS-FITC functionalized SNPs were prepared by adding 305 μL distilled H₂O and 50 μL TEOS to 5 mL of a 0.3 M Triton-X100/1.5 M1-hexanol/cyclohexane stock solution while vigorously stirring at room temperature. After 10 min thorough mixing, 100 μL of aqueous NH₄⁺H⁻ was added to initiate hydrolysis, and the resultant optically transparent microemulsion mixture was stirred for another 20 hrs before adding 100 μL of a 8 mg/mL solution of 3-[aminopropyl(trimethoxy)silyl]fluoresceine isothiocyanto methanolic solution and 2.0 μL 460 to 590 M_{W} 2-[methoxy-(polyethyleneoxy)propyl]trimethoxysilane to the reaction mixture and stirring for an additional 12 hrs. The functionalized SNPs were then precipitated with an equivalent volume of methanol (5 mL) and isolated via centrifuge at 12000 rpm for 20 min. The SNPs were subsequently washed twice with MeOH by redispursement via sonication and twice with H₂O before redispersing them in 5.0 mL of water.

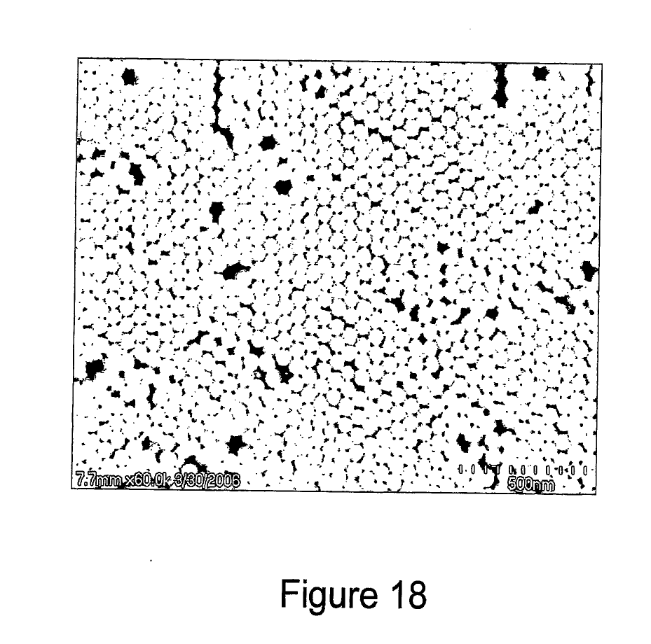

The functionalized SNPs were spherical with an outer diameter of approximately 50 nm as determined from SEM, and the bulk material was highly dispersible in aqueous solvent. A typical SEM image of PEG- and FITC-grafted nanospheres is shown in FIG. 18.

### Example 10

### Layer-by-Layer (LbL) deposition(s) onto [Silica+Ru(bpy)₃²⁺+DTTA-Gd³⁺]

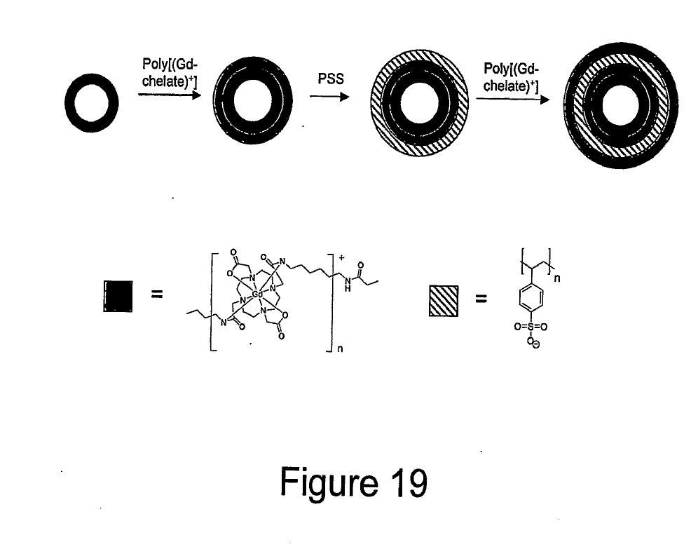

A scheme showing the synthesis of hybrid nanoparticles by a layer-by-layer (LbL) method is shown in FIG. 19.

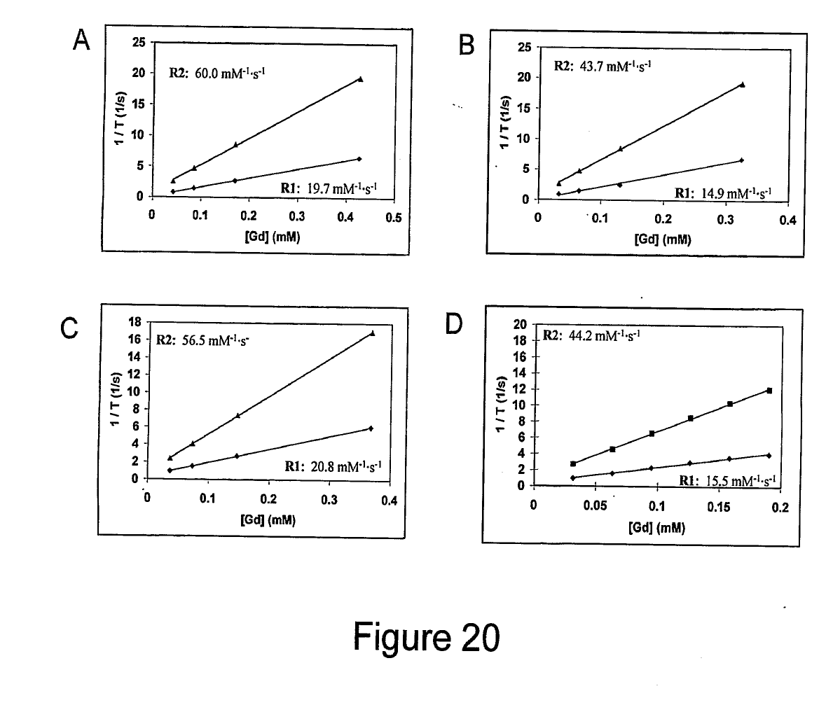

A 4 mL aqueous dispersion of negatively charged silica nanoparticles with mono(APS)DTTA-Gd (8.6 mg/mL dH₂O) is centrifuged at 12500 rpm for 30 minutes. The supernatant is removed, and replaced with 4 mL of positively charged poly[(Gd chelate)⁺] (1 mg/mL dH₂O). The chemical structure of a suitable poly[(Gd chelate)⁺] is shown at the bottom left of FIG. 19. The particles are dispersed, then vigorously ultrasonicated for 20 minutes to induce poly[(Gd chelate)⁺] absorption. The poly[Gd chelate)⁺] coated particles are centrifuged at 12500 rpm for 15 minutes. The supernatant is removed, and saved for further absorption cycles. The particles are dispersed in 4 mL dH₂O then centrifuged at 12500 rpm. This wash cycle (dispersion/centrifugation/decantation) is repeated twice. The thrice-washed single layered particles are dispersed in 4 mL of negatively charged PSS solution (1 mg/mL dH₂O) and ultrasonicated for 20 minutes. The PSS coated particles are subjected to three wash cycles and dispersed in 4 mL dH₂O. A 1 mL aliquot is removed and used to prepare sample for MR measurements without further purification (this corresponds to sample 3 in FIG. 20B). The remaining 3 mL are pelleted, placed into 3 mL of the recycled poly[(Gd chelate)⁺] solution, and ultrasonicated for 15 minutes and three wash cycles are performed. The particles are dispersed in 3 mL of fresh PSS solution (1 mg/mL dH₂O) and washed three times. A 1 mL aliquot is removed and used to prepare sample for MR measurements without further purification (this corresponds to the sample 4 in FIG. 20C). The remaining 2 mL is treated with the poly[(Gd chelate)⁺] solution and the PSS solution in a similar fashion as above. A 1 mL aliquot is removed and used for MR measurements without further purification without further purification (this corresponds to the sample 5 in FIG. 20D).

Throughout this process, successful deposition of poly[(Gd chelate)⁺] or PSS layer was suggested by the water dispersibility of the nanoparticles because of their different Zeta potentials. In particular, after treatment with poly[(Gd chelate)⁺], the particles become less dispersible in water. Treatment with PSS makes the particles more dispersible in water. UV-Vis spectroscopy was also used to follow the polyelectrolyte deposition process.

The r1 values of the particles of 3, 4, and 5 were determined to be 14.9, 20.8, and 15.5 s⁻¹ per mM of Gd³⁺ ions respectively. If it is assumed that complete ion exchange is achieved during each deposition process, the r1 values of the particles in samples 3, 4, and 5 can be estimated to be of the order of 4.2×10⁵, 8.9×10⁵, 8.8×10⁵ s⁻¹ per mM of particles, respectively. As indicated in FIGS. 20B, 20C, and 20D, very large r2 values were also observed for these nanoparticles.

### Example 11

### Synthesis of bis(2-aminoethylmethacrylate)diethylenetriamine pentaacetic acid

Diethylenetriamine pentaacetic acid dianhydride (0.0500 g, 0.1399 mmol) and 2-aminoethyl methacrylate (0.0487 g, 0.2939 mmol) were dissolved in 5 mL of anhydrous pyridine under nitrogen. The reaction was stirred under nitrogen for 18 hours. The product was then precipitated with copious amounts of hexanes, and collected via centrifugation at 3000 rpm for 10 minutes.

### Example 12

### Synthesis of Poly(acrylic acid)-based Nanomaterials

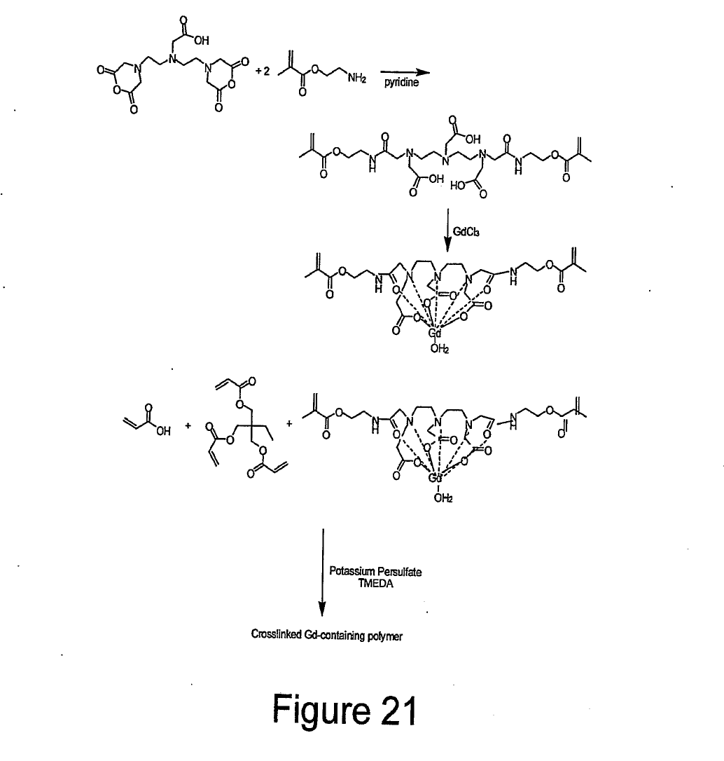

A scheme for the synthesis of nanomaterials comprising poly(acrylic acid) is shown in FIG. 21.

The polymerization of acrylic acid was carried out in a water-in-oil microemulsion. A 0.05 M cetyltrimethyl ammonium bromide (CTAB) solution was made in n-heptane with 1-hexanol as a cosurfactant. An aliquot of this solution was placed in a round bottom flask, and degassed with nitrogen for 10 minutes, while stirring vigorously. To this surfactant solution, an aqueous monomer solution was added which includes the monomer (acrylic acid), a Gd-chelating comonomer (DTPA bis(2-aminoethyl methacrylate)), a crosslinker (trimethylolpropane triacrylate, TMPTA), and a redox initiator (potassium persulfate). The volume of aqueous solution added was dependant on the desired w-value (w-=([H₂O]/[CTAB])), typically 5 to 15. After the aqueous solution was added the resultant microemulsion was degassed for an additional 5 minutes, while stirring vigorously. Tetramethylethane diamine (TMEDA) was then added to the microemulsion to initiate the polymerization. The reaction was then stirred at room temperature, under N₂, for 16 hours. The resulting polymer was then precipitated by the addition of ethanol, and the product was collected by centrifuging at 10000 rpm for 10 minutes, followed by washing with additional ethanol.

### Example 13

### Nanoparticles with Disulfide-Containing Functionalized Chelating Groups

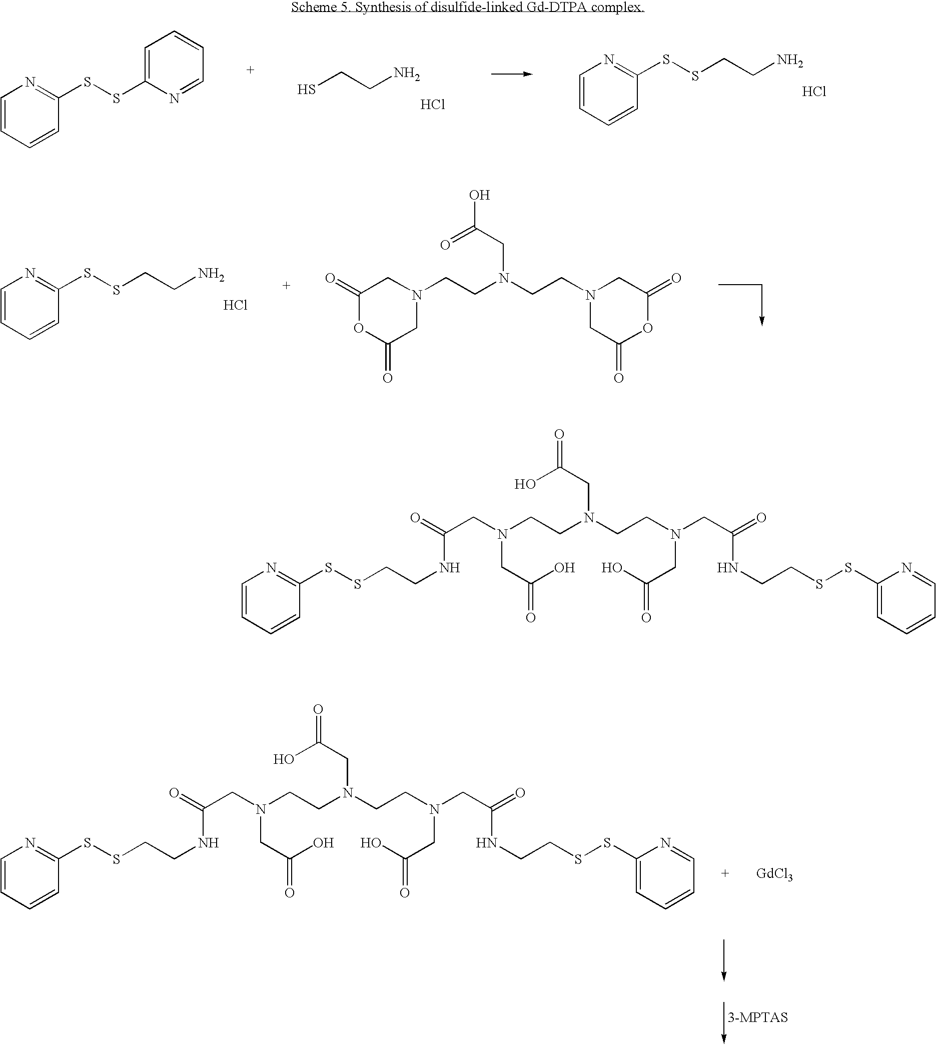

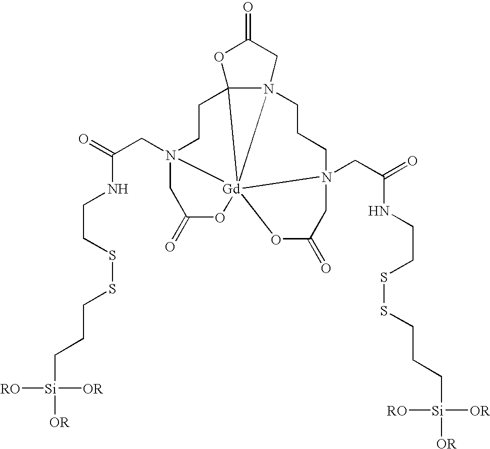

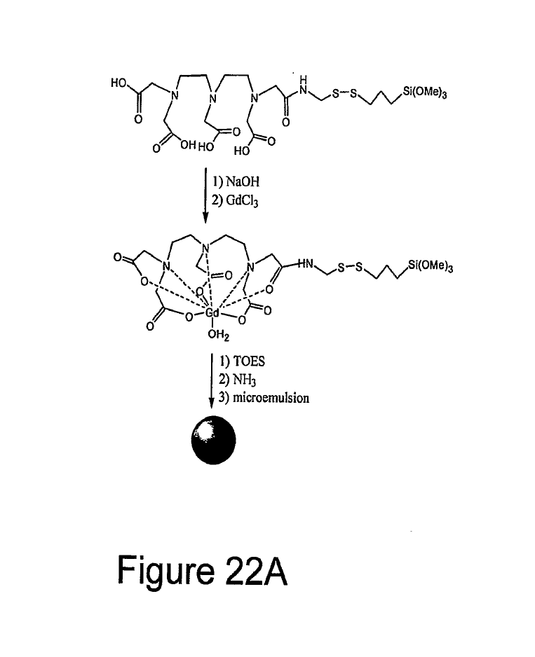

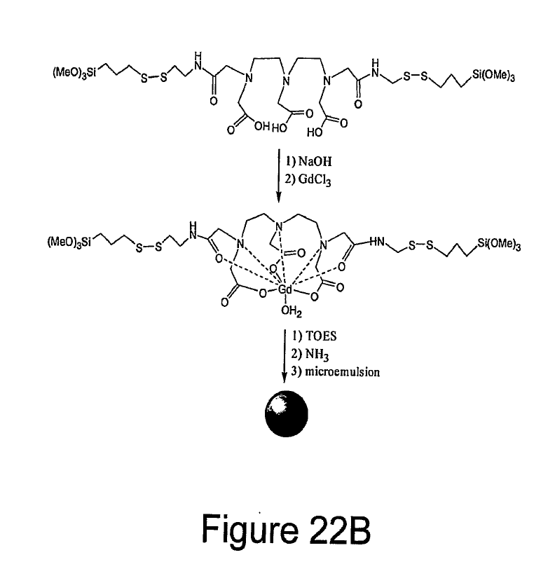

A synthetic route to a disulfide-containing Gd-DPTA complex is shown above in Scheme 5. FIGS. 22A and 22B show schemes illustrating how hybrid nanoparticles comprising functionalized chelating groups having biodegradable disulfide linkages can be prepared. More particularly, FIG. 22A shows a disulfide-comprising coordination complex group that has a single reactive siloxy group and a single linkage whose degradation can provide release of the chelating group from the nanoparticle. FIG. 22B shows the synthesis of a nanoparticle using a bis-disulfide containing functionalized chelating group comprising two reactive siloxy groups.

### Example 14

### Nanoparticle Imaging of Monocyte Cells

Cell Culture Studies: Monocyte immortalized lines were generated using the previously described methods of Monner (see Monner and Denker, J. Leukoc. Biol., 61(4), 469-480 (1997)) and Walker (see Walker, J. Immunol. Methods, 174, 25-31 (1994)) with minor modifications described by Lorenz et al. (Infect. Immun, 70, 4892-4896 (2002)). Briefly, bone marrow progenitor cells from C57Bl/6 mice were harvested and grown in conditioned medium containing 10% heat-inactivated fetal calf serum, 1% I-glutamine, and 20% LADMAC (catalog no. CRL 2420; American Type Culture Collection, Manassas, Va., United States of America) supernatant in Minimal Essential Medium. Once immortalized, cells were grown in the aforementioned conditioned medium, which provides the isolated monocytes with colony-stimulating factor-1. Cell lines were matured over 9 months to achieve a homogeneous population expressing the macrophage/monocyte marker MOMA-2 (data not shown) with phagocytic capacity.

Confocal imaging of labeled monocyte cells: Monocyte cells were incubated in media (2.0 mL) with nanoparticle suspension (17.0 μL, 24.6 mg mL⁻¹) for 30 minutes at 37° C. with 5% CO₂. The cells were isolated from the media by centrifugation at 1000 rpm for 10 min at 4° C., and subsequently washed with a fresh aliquot of media. The resulting isolated pellet was suspended in 100 μL of PBS and imaged using confocal microscopy: excitation at 488 nm, emission using 53 LP filter settings, and 252×zoom (63×oil immersion optical, 4×digital).

MTS cell viability assay: Monocyte cells were counted by trypan blue exclusion and distributed into a 96-well plate at a concentration of 5000 cells in 100 μL per well. Cells were incubated with various concentrations of 1: 123, 12.3, 1.23, 0.123, 0.0123, and 0 μg in 5 μL of distilled H₂O. After 20 h of incubation, 3-(4,5-dimethylthiazol-2-yl)-5-(3-carboxy-methoxyphenyl)-2-(4-sulfophenyl)-2H-tetrazolium (MTS) solution (20 μL) was added to each well and allowed to further incubate for 4 h. The microplate was read for 492 nm absorbance at time=0 h and time=4 h after MTS addition. The changes in absorbance (from t=0 h→t=4 h) were necessary to subtract nanoparticle background from the viability assay.

MRI Image Acquisition: Monocyte cells were trypsinized for 5 minutes at 37° C. and 5% CO₂ before collection by low speed centrifugation. Cell concentration was determined by the trypan blue exclusion assay. Approximately 18.1×10⁶ monocytes were placed in a culture dish with 1 mL of media and 0.433 mL of nanoparticle solution (24.6 mg/mL). After 1 hour of incubation, the cells were washed with fresh media twice and pelleted. A final layer of PBS (200 μL) was added on top, careful not to disturb the pellet, for MR imaging of the cells. Upon completion of MR imaging, the cells were digested in 1.0 M HNO₃ for DCP measurements of the total Gd³⁺ taken in by the cells.

### Example 15

### Results of Monocyte Cell Imaging Studies

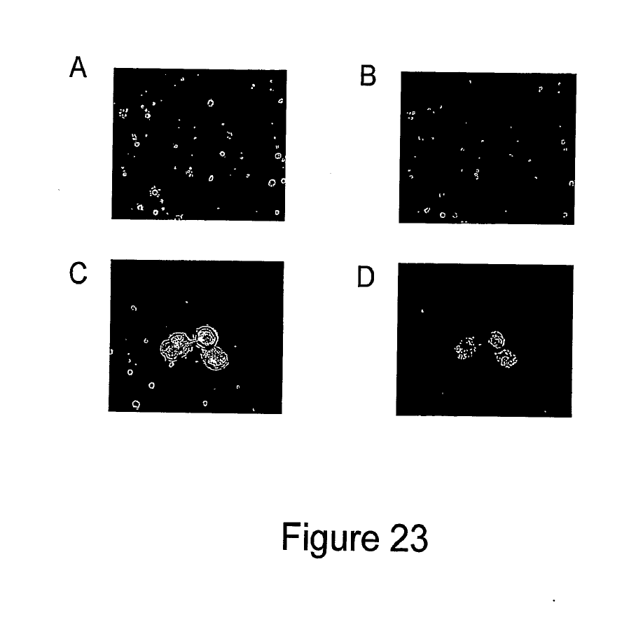

Preliminary imaging capability studies indicated that silica-based nanomaterials are non-toxic to monocyte and HeLa S3 cells at concentrations that would be adequate for significant MR image enhancement, as well as for optical imaging. By doping these silica particles or functionalizing them with fluorophores such as Ru(bpy)₃²⁺ or APS-FITC, they can be optically tracked during in vitro cell studies by fluorescence or confocal laser microscopy. Optical and confocal laser scanning fluorescence microscopic images of cellular uptake of SNPs by monocyte cells and HeLa S3 cells are shown in FIGS. 23A and 23B.

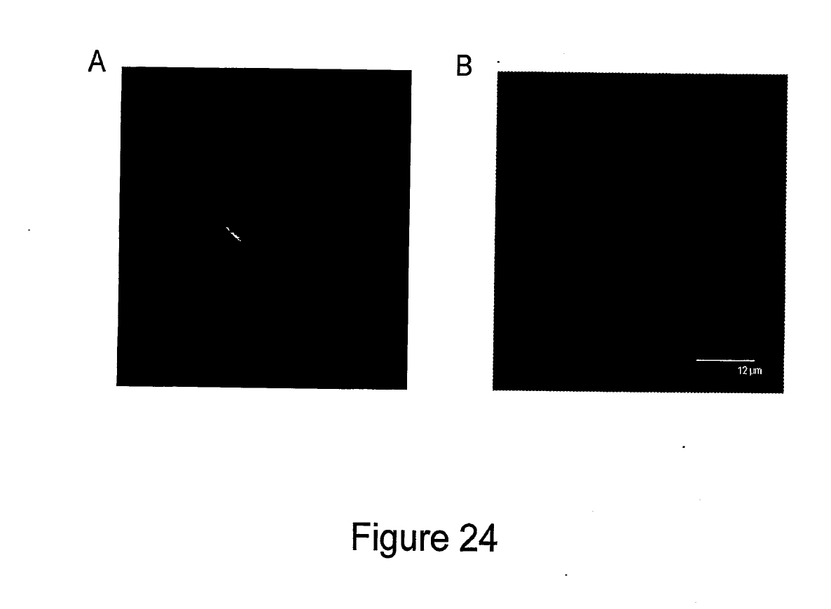

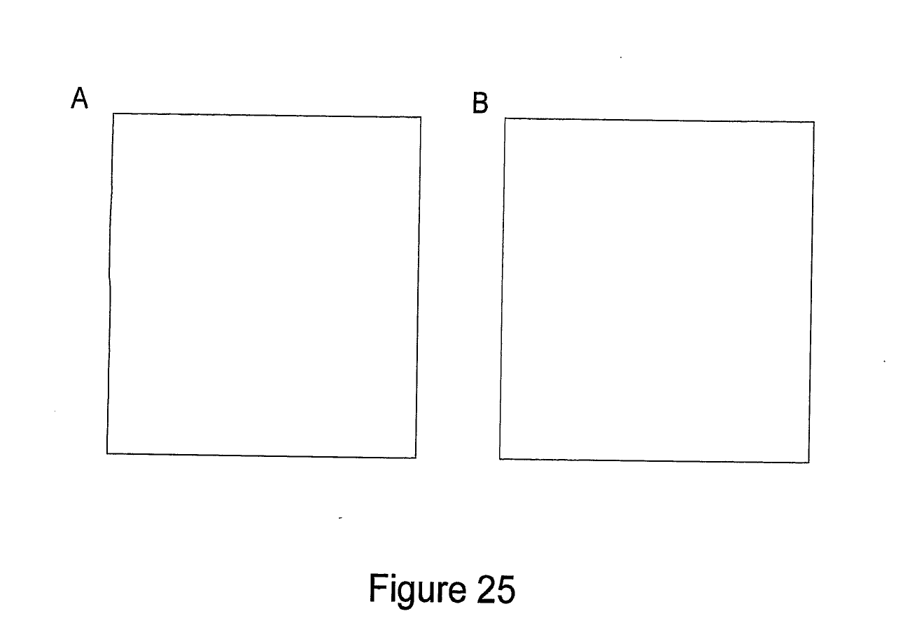

The Ru(bpy)₃²⁺-imbedded silica particles with mono(APS)DTTA-Gd coating were also further conjugated to anti-MHC-II antibody via an amide linkage. Optical and confocal laser scanning fluorescence images of monocyte cellular uptake of Ru(bpy)₃²⁺-imbedded silica particles with mono(APS)DTTA-Gd is shown in FIGS. 24A and 24B. Preliminary data suggested that the antibody-conjugated nanoparticles can bind to the cells surface, which expresses MHC-II receptors, in a frozen tissue slice that was obtained from an inflamed mouse intestine. See FIGS. 25A and 25B.

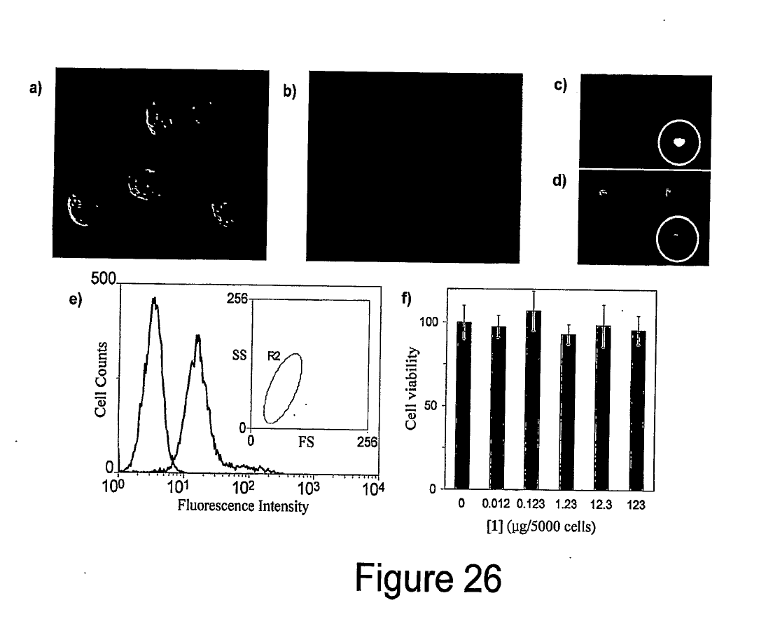

Further in vitro studies focused on the efficacy of nanoparticles of 1 as multimodal imaging contrast agents using an immortalized monocyte cell line. The monocyte cell line is of particular interest due to its phagocytic capacity as well as its important role in autoimmune diseases, such as rheumatoid arthritis. See Ma and Pope, Curr. Pharm. Des. 11, 569 (2005). The laser scanning confocal fluorescence microscopic studies clearly indicated the efficient uptake of 1 by monocyte cells after incubation with 2 mL of medium containing 0.42 mg of 1 for 0.5 hour. FIG. 26A and FIG. 26B show the optical and laser scanning confocal fluorescence microscopic images of monocyte cells labeled with 1. The ligand-to-metal charge transfer luminescence of [Ru(bpy)₃]Cl₂ is visible in the confocal z-section images.

As shown in FIG. 26E, monocyte labeling efficiency with 0.42 mg of 1 (per 1×10⁶ cells in 2 mL media) is greater than 98%. In labeling experiments using other amounts of 1, efficiency results were as follows: with 0.004 mg 1, labeling efficiency was 0.6%; with 0.042 mg 1, labeling efficiency was 10.8%; with 2.140 mg of 1, labeling efficiency was 99.4%.

The results of the MTS assay indicate that the nanoparticles of 1 were not toxic to monocyte cells. See FIG. 26F. The cells were completely viable even after incubation with a nanoparticle loading as high as 0.123 mg per 5000 monocyte cells.

Additionally, MRI studies show MR image enhancements for the labeled monocytes when compared with a control population of unlabeled monocyte cells. As shown in FIGS. 26C and 26D, significant positive signal enhancement in the T1-weighted image and negative signal enhancement in the T2-weighted image were observed in the labeled cells depending on the MR pulse sequence employed.

### Example 16

### In Vivo MR Imaging With Nanoparticles

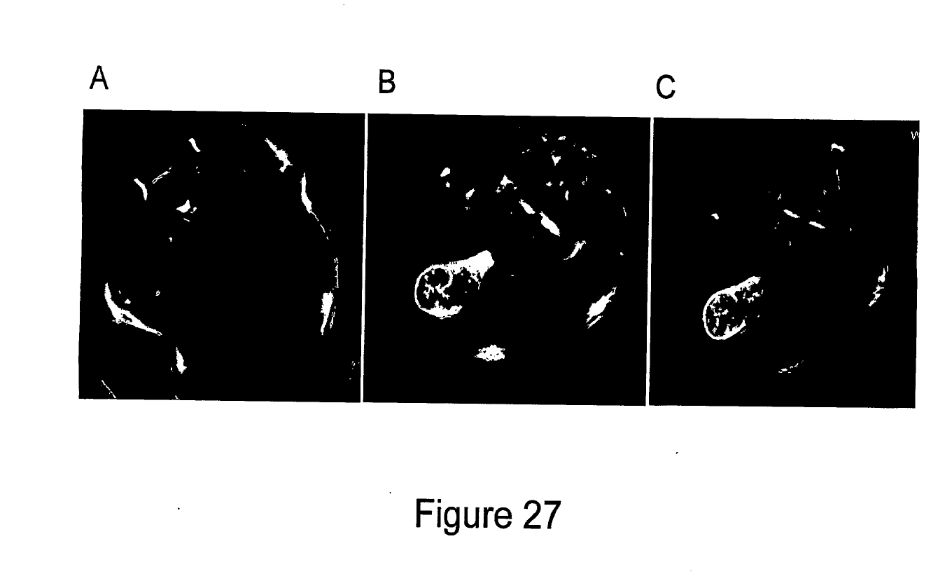

In vivo MR imaging was carried in genetically engineered mice with choroids plexus carcinoma (CPC). See, Brubaker et al. Cancer Res. 65, 8218-8223 (2005). Briefly, the CPC mouse was imaged with a spin-echo MR pulse sequence on a 3.0T scanner prior to the injection of the nanoparticle contrast agent to obtain a pre-contrast MR image. See FIG. 27A. Then, 25 mg of hybrid nanoparticles were injected to the CPC mouse via tail vein injection, and MR images were taken immediately after the injection (FIG. 27B) and 5 hours after the injection (FIG. 27C). Significant contrast enhancement was observed in the MR images taken immediately after the injection and 5 hours after the injection.

### Example 17

### Target-Specific Imaging of HT-29 Colon Cancer Cells

A peptide sequence containing arginine-glycine-aspartate (RGD) and seven consecutive lysines (K) were deposited onto the surface of LBL nanoparticles (which had also been doped with an optical imaging for fluorescence detection). The negatively-charged PSS layer electrostatically interacts with the positively-charged lysine residues to create a charge balanced assembly. The RGD sequence is thus displayed on the surface of the LbL nanoparticles, allowing the targeting of tumor cells that are known to overexpress integrin receptors.

For example, the RGD-displaying nanoparticles were used to label HT-29 tumor cells. HT-29 cells are human colon tumor cells that are known to overexpress integrin receptors (see Reinmuth et al., Cancer Res., 63, 2079-2087 (2003); and Lee and Juliano, Mol. Biol. Cell, 11, 1973-1987 (2000)) and have been previously labeled with K₇RGD (SEQ ID NO: 1) peptide ligands electrostatically decorated onto microspheres. See Toublan et al., J. Am. Chem. Soc., 128, 3472-23473 (2006).

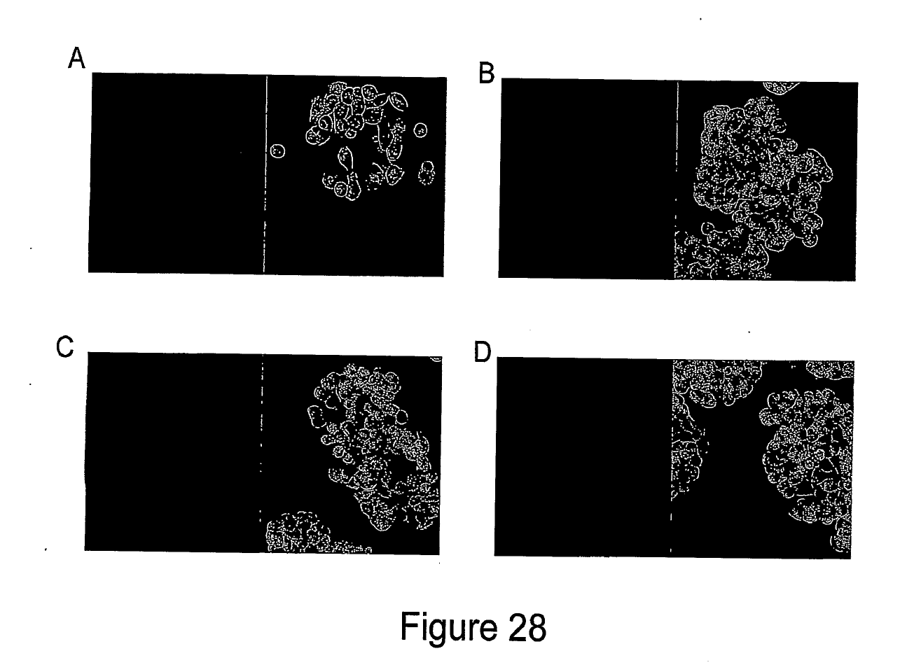

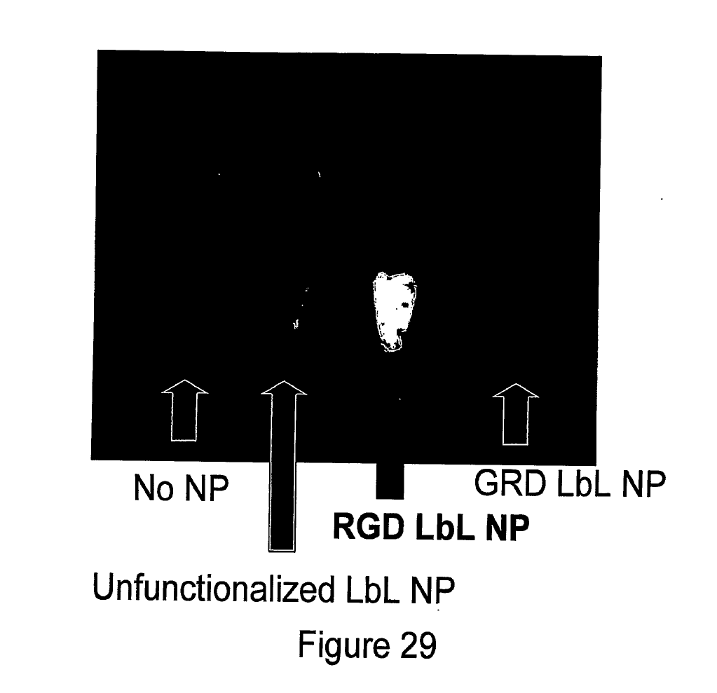

Both optical imaging (FIG. 28B) and MR imaging (FIG. 29, sample second to the right) demonstrated that RGD-functionalized nanoparticles are specifically targeted to the HT-29 colon cancer cells. In comparison, LbL nanoparticles comprising a scrambled sequence of K₇GRD displayed on its surface showed diminished labeling capabilities toward HT-29 cells. See FIG. 28D and FIG. 29 (the sample on the right). Unfunctionalized LbL nanoparticles (i.e., those having no surface-associated targeting group) showed little to no detectable labeling. See FIG. 28C and FIG. 29 (sample second from the left). Images of HT-29 cells with no nanoparticles are also shown. See FIG. 28A and FIG. 29 (sample on the left).

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A contrast agent for magnetic resonance imaging (MRI) comprising a hybrid nanoparticle, said hybrid nanoparticle comprising:
a polymeric matrix material; and
a plurality of coordination complexes, each coordination complex comprising a functionalized chelating group and a paramagnetic metal ion.

2. The contrast agent of claim 1, comprising at least one luminophore for optical imaging.

3. The contrast agent of claim 2, wherein the luminophore is a fluorophore selected from the group consisting of ruthenium(II) tris(2,2′-bipyridine) (Ru(bpy)₃²⁺), fluoroscein isothiocyanate (FITC), a semiconducting quantum dot, and a doped semiconducting quantum dot.

4. The contrast agent of claim 2, wherein the luminophore is embedded in the hybrid nanoparticle.

5. The contrast agent of claim 2, wherein the luminophore is bound to a surface of the hybrid nanoparticle.

6. The contrast agent of claim 1, wherein the polymeric matrix material is an inorganic polymer.

7. The contrast agent of claim 6, wherein the inorganic polymer comprises silicon.

8. The contrast agent of claim 7, wherein the inorganic polymer material comprises SiO₂.

9. The contrast agent of claim 1, wherein the polymeric matrix material comprises an organic polymer.

10. The contrast agent of claim 9, wherein the organic polymer is selected from the group consisting of polyacrylic acid and polylactide.

11. The contrast agent of claim 1, wherein the polymeric matrix material is biodegradable.

12. The contrast agent of claim 1, wherein the polymeric matrix material is non-biodegradable.

13. The contrast agent of claim 1, wherein the paramagnetic metal ion comprises an element selected from the group consisting of a transition element, a lanthanide and an actinide.

14. The contrast agent of claim 13, wherein the paramagnetic metal ion comprises an element selected from the group consisting of scandium, titanium, vanadium, chromium, manganese, iron, cobalt, nickel, copper, molybdenum, ruthenium, cerium, praseodymium, neodymium, promethium, samarium, europium, gadolinium, terbium, dysprosium, holmium, erbium, thulium, and ytterbium.

15. The contrast agent of claim 14, wherein the paramagnetic metal ion is selected from the group consisting of gadolinium(III) and manganese(II).

16. The contrast agent of claim 1, wherein the functionalized chelating group comprises a polyaminocarboxylate metal chelating ligand or a polyaminophosphonate metal chelating ligand.

17. The contrast agent of claim 16, wherein the metal chelating ligand comprises a ligand selected from the group consisting of diethylenetriamine tetraacetate (DTTA), diethylenetriamine pentaacetate (DTPA), and 1,4,7,10-tetraazacyclododecane-1,4,7,10-tetraacetic acid (DOTA).

18. The contrast agent of claim 1, wherein the functionalized chelating group is functionalized by at least one reactive moiety that can covalently bond to the polymeric matrix material or to another functionalized chelating group.

19. The contrast agent of claim 18, wherein the at least one reactive group is selected from the group consisting of vinyl, siloxy, and combinations thereof.

20. The contrast agent of claim 18, wherein the functionalized chelating group is functionalized by more than one reactive moiety.

21. The contrast agent of claim 18, wherein the functionalized chelating group is selected from aminopropyl(trimethoxysilyl)diethylenetriamine tetraacetate, bis(aminopropyltriethoxysilyl)diethylenetriamine pentaacetate, bis(2-aminoethyl-methacrylate)diethylenetriamine pentaacetic acid, bis(aminopropyltrimethoxysilyl)-1,4,7,10-tetraazacyclododecane-1,4,7,10-tetraacetic acid, and aminopropyl-(trimethoxysilyl)-1,4,7,10-tetraazacyclododecane-1,4,7,10-tetraacetic acid.

22. The contrast agent of claim 1, wherein the functionalized chelating group comprises at least one biodegradable linkage.

23. The contrast agent of claim 22, wherein the biodegradable linkage is disulfide.

24. The contrast agent of claim 1, wherein the polymeric matrix material and the plurality of coordination complexes form a copolymer.

25. The contrast agent of claim 24, wherein the plurality of functionalized coordination complexes are dispersed throughout the copolymer.

26. The contrast agent of claim 24, wherein the plurality of coordination complexes form a polymeric layer disposed over a core polymeric layer comprising the polymeric matrix material.

27. The contrast agent of claim 1, wherein one or more of the plurality of coordination complexes is bound to a surface of the nanoparticle.

28. The contrast agent of claim 1, wherein the nanoparticle comprises one or more additional anionic groups.

29. The contrast agent of claim 28, wherein the one or more additional anionic groups comprise sulfonate groups.

30. The contrast agent of claim 1, wherein the nanoparticle further comprises a layer comprising anionic groups.

31. The contrast agent of claim 30, wherein the layer comprises poly(styrene sulfonate) (PSS).

32. The contrast agent of claim 1, wherein the contrast agent further comprises a plurality of layers comprising:
a first layer comprising the polymeric matrix material and at least some of the plurality of coordination complexes; and
a second layer disposed over the first layer, said second layer comprising at least some of the plurality of coordination complexes.

33. The contrast agent of claim 32, further comprising a third layer disposed over the second layer, said third layer comprising anionic groups.

34. The contrast agent of claim 33, wherein the third layer comprises poly(styrenesulfonate) (PSS).

35. The contrast agent of claim 33, further comprising a fourth layer disposed over the third layer, said fourth layer comprising at least some of the plurality of coordination complexes.

36. The contrast agent of claim 35, further comprising one or more additional layers comprising some of the plurality of coordination complexes and one or more additional layers comprising anionic groups, said additional layers being disposed such that:
each layer comprising some of the plurality of coordination complexes is the outermost layer of the nanoparticle and is disposed over a layer of anionic groups or is an inner layer of the nanoparticle and is disposed between two layers of anionic groups; and
each layer comprising anionic groups is either the outermost layer of the nanoparticle and is disposed over a layer comprising some of the plurality of coordination complexes or is an inner layer of the nanoparticle and is disposed between two layers, each comprising some of the plurality of coordination complexes.

37. The contrast agent of claim 1, wherein the nanoparticle is spherical.

38. The contrast agent of claim 37, wherein the nanoparticle has a diameter of about 100 nm or less.

39. The contrast agent of claim 38, wherein the nanoparticle has a diameter of about 50 nm or less.

40. The contrast agent of claim 1, further comprising an additional moiety bound to a surface of the nanoparticle, said additional moiety selected from the group consisting of a targeting agent, a solubility-enhancing agent, a circulation half-life enhancing agent, and a combination thereof.

41. The contrast agent of claim 40, wherein the additional moiety is a targeting agent selected from the group consisting of an antibody or an antibody fragment.

42. The contrast agent of claim 41, wherein the targeting agent is an anti-major histocompatibility complex (MHC)-II antibody.

43. The contrast agent of claim 40, wherein the additional moiety is a targeting agent that targets a tumor.

44. The contrast agent of claim 40, wherein the additional moiety comprises a polyethylene glycol (PEG)-based polymer.

45. The contrast agent of claim 44, wherein the PEG-based polymer is polyethylene oxide (PEO)-500.

46. The contrast agent of claim 1, wherein the nanoparticle comprises at least one thousand paramagnetic metal ions.

47. The contrast agent of claim 46, wherein the nanoparticle comprises at least 25,000 paramagnetic metal ions.

48. The contrast agent of claim 47, wherein the nanoparticle comprises at least 60,000 paramagnetic metal ions.

49. The contrast agent of claim 1, wherein the contrast agent has a longitudinal relaxivity (r1) of about 7.0 mmol⁻¹s⁻¹ or greater, calculated based on metal ion concentration.

50. The contrast agent of claim 49, wherein the contrast agent has a r1 of about 19.7 mmol⁻¹s⁻¹ or greater, calculated based on metal ion concentration.

51. The contrast agent of claim 1, wherein the contrast agent has a longitudinal relaxivity (r1) of about 2×10⁵ mmol⁻¹ s⁻¹ or greater, calculated based on nanoparticle concentration.

52. The contrast agent of claim 51, wherein the contrast agent has a r1 of about 4.9×10⁵ mmol⁻¹ s⁻¹ or greater, calculated based on nanoparticle concentration.

53. The contrast agent of claim 1, wherein the contrast agent has a transverse relaxivity (r2) of about 10 mmol⁻¹ s⁻¹ or greater, calculated based on metal ion concentration.

54. The contrast agent of claim 53, wherein the contrast agent has a r2 of about 60 mmol⁻¹s⁻¹ or greater, calculated based on metal ion concentration.

55. The contrast agent of claim 1, wherein the contrast agent has a transverse relaxivity (r2) of about 6.1×10⁵ mmol⁻¹ s⁻¹ or greater, based on nanoparticle concentration.

56. The contrast agent of claim 55, wherein the contrast agent has a r2 of about 7.8×10⁵ mmol⁻¹ s⁻¹ or greater, based on nanoparticle concentration.

57. A formulation comprising:
a hybrid nanoparticle, wherein the hybrid nanoparticle comprises a polymeric matrix material and a plurality of coordination complexes, each coordination complex comprising a functionalized chelating group and a paramagnetic metal ion; and
a pharmaceutically acceptable carrier.

58. The formulation of claim 57, wherein the hybrid nanoparticle further comprises a luminophore.

59. The formulation of claim 57, wherein the pharmaceutically acceptable carrier is pharmaceutically acceptable in humans.

60. A method of imaging one of a cell, a tissue, and a subject, the method comprising:
administering to one of a cell, a tissue, and a subject a contrast agent, said contrast agent comprising a hybrid nanoparticle, said hybrid nanoparticle comprising:
a polymeric matrix material; and
a plurality of coordination complexes, each coordination complex comprising a functionalized chelating group and a paramagnetic metal ion; and
rendering a magnetic resonance image of the one of a cell, a tissue, and a subject.

61. The method of claim 60, wherein the hybrid nanoparticle further comprises a luminophore.

62. The method of claim 61, wherein the method further comprises optically imaging the contrast agent.

63. A method of detecting a disease state in one of a cell, a tissue, and a subject, said method comprising:
administering to one of a cell, a tissue, and a subject a contrast agent, said contrast agent comprising a hybrid nanoparticle, said hybrid nanoparticle comprising:
a polymeric matrix material; and
a plurality of coordination complexes, each coordination complex comprising a functionalized chelating group and a paramagnetic metal ion; and
rendering a magnetic resonance image of the one of a cell, a tissue and a subject, thereby detecting a disease state in the one of a cell, a tissue and a subject.

64. The method of claim 63, wherein the disease state is selected from one of cancer, cardiovascular disease, and a disease associated with inflammation.

65. The method of claim 63, wherein the disease state is rheumatoid arthritis.

66. The method of claim 63, wherein the subject is a human.

67. A method of synthesizing a hybrid nanoparticle, said hybrid nanoparticle comprising a polymeric matrix material and a plurality of coordination complexes, each of the plurality of coordination complexes comprising a functionalized chelating group and a paramagnetic metal ion, the method comprising:
(a) providing a first mixture comprising a water-in-oil microemulsion system comprising water, an organic solvent, a surfactant, and a co-surfactant;
(b) adding a polymerizable monomer and a plurality of coordination complexes, each of said plurality of coordination complexes comprising a functionalized chelating group and a paramagnetic metal ion, to the first mixture to form a second mixture;
(c) mixing said second mixture for a first period of time;
(d) adding a polymerization agent to the second mixture to form a third mixture; and
(e) mixing the third mixture for a second period of time to form a hybrid nanoparticle.

68. The method of claim 67, further comprising precipitating the hybrid nanoparticle by adding an alcohol to the third mixture.

69. The method of claim 67, wherein the surfactant is a non-ionic surfactant.

70. The method of claim 69, wherein the surfactant is Triton-X100.

71. The method of claim 70, wherein the co-surfactant is 1-hexanol.

72. The method of claim 71, wherein the molar ratio of Triton-X100 to 1-hexanol ranges between about 1 and about 5.

73. The method of claim 67, wherein the polymeric matrix material is an inorganic polymer.

74. The method of claim 73, wherein the polymerizable monomer is tetraethyl orthosilicate (TEOS).

75. The method of claim 73, wherein the water to surfactant ratio of the third mixture ranges from about 10 to about 25.

76. The method of claim 73, wherein the polymerization agent is aqueous ammonia.

77. The method of claim 67, wherein the polymeric matrix material is an organic polymer.

78. The method of claim 77, wherein the polymerizable monomer comprises acrylic acid.

79. The method of claim 77, wherein the plurality of coordination complexes each comprise a functionalized chelating group comprising bis(2-aminoethylmethacrylate)diethylenetriamine pentaacetic acid.

80. The method of claim 77, wherein step (b) further comprises adding a cross-linker.

81. The method of claim 80, wherein the cross-linker comprises trimethylolpropane triacrylate (TMPTA).

82. The method of claim 77, wherein step (b) further comprises adding a redox initiator.

83. The method of claim 82, wherein the redox initiator is potassium persulfate.

84. The method of claim 77, wherein the polymerization agent is tetramethylethane diamine (TMEDA).

85. The method of claim 77, wherein the surfactant is cetyltimethyl ammonium bromide (CTAB).

86. The method of claim 77, wherein the microemulsion has a water to surfactant ratio ranging from about 5 to about 15.

87. The method of claim 67, wherein step (b) further comprises adding a luminophore to the first mixture as part of forming the second mixture.

88. The method of claim 87, wherein the luminophore comprises ruthenium(II) tris(2,2′-bipyridine) (Ru(bpy)₃²⁺).

89. The method of claim 67, further comprising adding one or more surface functionalization moiety to the third mixture after the second period of time, thereby forming a fourth mixture, and mixing the fourth mixture for a third period of time to form a surface functionalized hybrid nanoparticle.

90. The method of claim 89, wherein the one or more surface functionalization moiety comprises a luminophore, a hydrophilic polymer, a group that can serve as a linker between the hybrid nanoparticle and a targeting moiety, a coordination complex comprising a functionalized chelating group and a paramagnetic metal ion, and combinations thereof.

91. The method of claim 89, wherein the one or more surface functionalization moiety is selected from the group consisting of 3-[aminopropyl(trimethoxy)silyl]fluoresceine isothiocyanate, and 2-[methoxy-(polyethyleneoxy)propyl]trimethoxysilane.

92. A method of synthesizing a hybrid nanoparticle, said hybrid nanoparticle comprising a polymeric matrix material and a plurality of coordination complexes, each of the plurality of coordination complexes comprising a functionalized chelating group and a paramagnetic metal ion, further wherein one or more of the plurality of coordination complexes is bound to a surface of the hybrid nanoparticle, the method comprising:
(a) providing a first mixture comprising a water-in-oil microemulsion system comprising water, an organic solvent, a surfactant and a co-surfactant;
(b) adding a polymerizable monomer to the first mixture to form a second mixture;
(c) mixing said second mixture for a first period of time;
(d) adding a polymerization agent to the second mixture to form a third mixture;
(e) mixing the third mixture for a second period of time;
(f) adding to the third mixture a plurality of coordination complexes, each of the plurality of coordination complexes comprising a functionalized chelating group and a paramagnetic metal ion to form a fourth mixture; and
(g) mixing the fourth mixture for a third period of time to form a hybrid nanoparticle having one or more of the plurality of coordination complexes bound to a surface of the hybrid nanoparticle.

93. The method of claim 92, wherein step (b) further comprises adding a luminophore to the first mixture as part of forming the second mixture.

94. The method of claim 93, wherein the luminophore comprises ruthenium(II) tris(2,2′-bipyridine) (Ru(bpy)₃²⁺).

95. The method of claim 92, further comprising adding an alcohol to the fourth mixture after the third period of time, thereby precipitating the hybrid nanoparticle.

96. A method of synthesizing a layered hybrid nanoparticle, said layered hybrid nanoparticle comprising a polymeric matrix material and a plurality of coordination complexes, each of the plurality of coordination complexes comprising a functionalized chelating group and a paramagnetic metal ion, the method comprising:
(a) preparing a hybrid nanoparticle in a water-in-oil microemulsion, said hybrid nanoparticle comprising a polymeric matrix material and a plurality of coordination complexes, each of the plurality of coordination complexes comprising a functionalized chelating group and a paramagnetic metal ion; and
(b) adsorbing onto the hybrid nanoparticle prepared in step (a) a polymer comprising additional coordination complexes, said additional coordination complexes each comprising a functionalized chelating group and a paramagnetic metal ion to form a layer of polymerized coordination complexes over the surface of the hybrid nanoparticle.

97. The method of claim 96, wherein the adsorbing of step (b) comprises providing ultrasonication to a mixture of the hybrid nanoparticle and the polymer comprising additional coordination complexes.

98. The method of claim 96, further comprising contacting the layered hybrid nanoparticle with a mixture of an anionic polymeric material, said anionic polymeric material forming a layer over the layer of polymerized coordination complexes.

99. The method of claim 98, wherein the anionic polymeric material is poly(styrene sulfonate) (PSS).

100. The method of claim 98, further comprising adding one or more additional layers to the layered hybrid nanoparticle such that the one or more additional layers are alternately a layer comprising polymeric coordination complex and a layer comprising anionic polymeric material.


SEQUENCE LISTING (1 sequence)

SEQ ID NO: 1 - protein, 10 aa, Artificial sequence
  Artificially synthesized peptide ligand
KKKKKKKRGD